SEQUENCE LISTING (20 sequences)

SEQ ID NO: 1 - DNA, 1551 bp, Dehalococcoides sp.
atgcataatttccattgtacgataagtaggcgagattttatgaagggattggggttagcgggagcagggataggtgccgcgacttcagttatgccgaattttcacgacttggatgaagtaatttctgctgctagtgccgaaaccagttctttgtcgggtaaatctcttaataattttccttggtatgtgaaagaaagggattttgaaaatcctaccattgatatagattggtctatacttgcgcgtaatgacggttacaatcatcagggagcctattggggacctgtacctgaaaatggagatgataaaaggtatcctgatcccgcggaccagtgtcttactctaccagaaaagagagatctttatttagcgtgggcaaaacagcaatttcctgactgggaaccaggaattaatggccatgggccaacaagggacgaagctttatggtttgcctcaagtacaggtggtatcggtaggtatagaattcctggtacccagcaaatgatgtccacaatgcgtcttgacgggtctactggtggttggggttatttcaatcaaccaccggcagcagtctggggagggaaatacccaaggtgggaaggaactcctgaagagaatacgttgatgatgcgaactgtttgtcaattttttggttactccagtataggtgtaatgccaatcaccagcaatacaaagaagcttttttttgaaaagcaaatacctttccaatttatggctggagatcccggtgtatttgggggaacgggaaatgtgcagtttgatgtcccgctgccaaagacacctgttccaatagtctgggaggaagtcgataaagggtattataatgaccagaaaattgtaatacccaataaggctaactgggtattaacaatgacaatgcctttaccagaagatcgttttaaacgttctctagggtggtcacttgacgcttcaagtatgattgcctatcctcagatggcttttaatggaggccgagttcagacttttttaaaagcacttggctatcaaggacttggtggcgacgtggctatgtggggacctggtggtgcttttggagttatgagtggtctttccgaacaaggtcgtgctgctaatgaaatcagccccaaatacggttcggcaactaagggctctaatcgattagtttgtgatttgcccatggttccgaccaagccaattgatgctggcatacacaaattctgtgaaacgtgtggcatttgtacaacagtttgtccctcaaatgctatccaggtaggtcctccacaatggagtaataatcggtgggataatacccctggttatcttggttatcgacttaactggggtagatgtgttctttgtacaaactgtgagacctattgcccattttttaacatgactaatggttctttgattcataacgtagtcagatccacagttgcagctacaccggtttttaattcatttttccgccaaatggaacatacatttggatatggtatgaaagatgatttaaacgattggtggaatcaatcacacaagccttggtaa

SEQ ID NO: 2 - DNA, 2087 bp, Dehalococcoides sp.
ttcaggaggcatgttatggatacttggcttgcggactaccgcatgacaggcgccaaagtgcgaaaagctttttttaaatgaggtattttaaaagtatgagtgaaaaatatcattctacagtcacaaggcgtgatttcatgaagagattaggtttggcaggagccggtgcgggggcactgggtgccgcagtacttgcagagaataacctgccgcatgagtttaaagatgttgatgacctgctgtcagcaggtaaagctttagagggtgaccacgctaataaagtaaacaatcatccatggtgggttaccacgcgtgatcatgaggatccaacctgtaatatagattggagccttataaaaagatacagcggttggaacaaccagggagcatacttcttacctgaggattacctgtctccaacctatacaggtagaagacataccattgttgattcaaaactagaaatagaattacagggtaaaaaataccgtgatagtgcctttatagaatcaggcatagactggatgaaggaaaatattgatccagattatgaccctggtgaactgggctatggcgaccgcagggaagatgccctaatatatgccgccacgaatggctcacataattgctgggagaacccgctttatggacgctatgaaggttctaggccttatctctctatgcgaaccatgaatggaataaacggcttgcatgaatttggtcacgcagatatcaaaaccaccaactacccgaagtgggagggtacgcctgaagagaacctgttaatcatgcgcaccgccgcgcgctacttcggggcttcttccgttggcgccattaagataacggataacgtgaagaaaatcttctatgccaaagcccagcccttttgcctcgggccttggtatacgattacaaatatggctgaatacattgaatatccggtcccagtagataattatgctatacccattgtgtttgaagatatccctgcagaccagggacactacagctacaaacgctttggcggtgatgataagatagcagtacccaatgcactggataacatcttcacctataccatcatgctccctgagaagcgctttaaatatgcacactctatacctatggacccatgctcttgtattgcctatcccctctttacagaggttgaggcacgcattcagcaattcattgcaggccttggctataactcgatgggtggtggagttgaagcttggggtccgggcagtgccttcggcaacttaagtggccttggggaacaatcacgcgtatcaagcactattgagccccgctacggttccaacaccaagggttccctaaggatgcttaccgacctgcctcttgcccccaccaagcctatagatgccggtatccgtgagttctgtaagacctgcggcatctgtgccgagcattgtcctacccaagctatctcgcatgaagggccgcgctatgactcaccacactgggattgcgtaagcggttatgagggctggcaccttgactatcacaaatgcactaactgtaccatctgtgaggccgtctgccccttcttcactatgagcaataactcctgggtgcacaacttggtcaagtccactgttgccactacgcccgtttttaacggtttctttaagaatatggaagaagccttcggctacggcccgcgctactcaccaagcagggatgaatggtgggcctcagaaaacccaatacgcggcgcaagcgtagatattttttaagagaaaggatggaatagattattatgggtggtgctttatactactttttagtcggtatgcttattggcggtgccgcaatatggtttattacctatacccagtttaagaatataagttttaaatggtgggaatggtcccttatggcgctaagcctgcttttggtatcctctatcttccagcatatgtattcttctatgagcgtggagatggagtatcagagtgcctttatgtaccttggcgtctttggcacactggctgtcatcttaaaccttattgtatggcgcacatatagcggcaggaaggaataaaatcttttgatccgcaatg

SEQ ID NO: 3 - DNA, 2794 bp, Dehalococcoides sp.
aattactgggtgtacctgactatgacgcggctcaagaagccgggaatcttattcttaacctgtcatcgctctggtccaaggccaacttatcagagcagagaaagatactccttaccacgctggacggggtctatgtggatgtgaaagagcatcgctcggtaatagcagttaaagccaagccacccttcaggcctatctttcaggtggcagtttcaaagaaagaatctaagattcatattttaaacgagccattaagtcatgaacctagcggctcgtccgtgtttctggtggagacgggggagggttggactctccctgaaacaatggtgatggggtttgcaggatgttaagatgcaaaaataactgtagtttttattatttaaagtaactcgtgttcgatatgtagtaaattagcaaagactagtacttaagtattaatacttaagtacatttattttatatatctttgcgtattttgtgcaattgacattactatataaaatgctagaatacaaataatcaactgtgtatttgtactgaggaaacgcttatggatatttggcgttcaggaaagactgaatggctctagggaaagacctaaatatatctttaggataaatttatgagtaaatttcataaaacgattagccgccgagatttcatgaaaggactaggattagccggggcaggcataggcgctgttgcggcgtcagctccggtttttcatgacattgatgaacttgtttcaagcgaagcaaattctactaaagatcaaccttggtacgttaagcatcgagagcattttgaccctacgattacagttgactgggatatttttgatagatatgacgggtatcagcataagggtgtctatgaaggccctccagatgctccctttacatcatggggcaataggcttcaggtgagaatgtcaggtgaagagcaaaagaagcgaattttggccgctaaaaaagagaggttccctggttgggacggtgggttacacgggagaggggatcagaggggatcagcactattttacgcagtaactcaaccatttcctggtagtggtgaggaagggcacggactattccaaccttatcctgatcaacccggtaagttttacgcgagatggggtttgtatggtccgccacatgattcagcgccacctgatgggagcgtaccaaaatgggagggtactccagaagacaattttctaatgctgagggcagctgcaaaatattttggtgctggtggcgttggtgctcttaacctggcagatcccaaatgcaaaaaactaatatataagaaagctcagccgatgactctaggaaaaggaacatacagtgaaataggtggaccaggaatgatcgatgcaaaaatttatcccaaggttcctgaccatgccgtacctattaactttaaggaagcggattatagctactacaatgatgcagagtgggttattccaacaaagtgtgaatccattttcactttcaccctacctcaaccacaagaactcaataagaggacgggtggtatagcaggtgctggatcatatactgtatacaaagatttcgctagggtaggcactttagtccaaatgtttattaagtatctaggttatcacgctttatattggccaattggatggggaccgggtggttgctttaccacttttgacgggcaaggtgaacagggtagaacaggtgctgctatccattggaagtttggttcttcacaacgtggttctgaaagagtaataactgatttaccgatagctcctaccccgccaattgatgcaggtatgtttgagttttgcaaaacctgttatatatgccgtgacgtttgcgtctctgggggtgtgcaccaagaagacgaaccaacttgggattcaggtaattggtggaatgtacaaggatatctcggctaccgaacggattggagtggttgccataaccagtgcggtatgtgtcaatcctcctgcccttttacttatttaggtttggaaaatgcttcattagtgcacaaaatagtaaaaggtgttgttgctaacacgactgtttttaatagtttttttaccaatatggagaaagcattaggatatggtgatttaaccatggaaaattctaactggtggaaagaagaaggaccgatatacggctttgatcccggtacttagaaatagatactaaattcgatagaaaataaaggaaattgaaatggatgctatatattttttcttaacaattgcattagcagttggactaactatgctatttacctggtttaaaaagaataatatcactttaaagtggaatgagtgggtacttggcatattggggctgttactagctttgtttgctattcaacacacatatgccagtgctacatatgaatttgaatatacgtcagcatggatagtgggcgtcatagtgttattgttagctgtagtaccgttgttatttgcggcaagatcagtaagacgcagggtagacaaataacgggtatctttaagtagataaggtagtaagtcgcttgggttgcacgcttaataaagcaatcaagagctatttcataaaagttaacccccgagttgtattacccgtggcaactcgggggattatagttttcttttatgtgcaaatataacgagaaaatggatacaaatagacagtgaagaagtttattattgcaattattgcgctggtcttattgggcgccggcatatatggaaataccggcggcgatcaagatattgatgcgtatctagctgaagctttacctgaagcgc

SEQ ID NO: 4 - DNA, 1512 bp, Dehalococcoides sp.
  misc_feature (1431)..(1431): n is a, c, g, or t
aggtgatccagccgcagcttccgctacggctaccttgttacgacttcgtcccaattaccagtcccaccctcggcgactgcctccttgcggttggcacatcaacttcaagtgttaccggctttcatgacgtgacgggcggtgtgtacaaggcccgagaacgtattcaccgcaccttgctgatatgcggttactagcaactccaacttcatgcaggcgggtttcagcccgcaatccgaactgaggacagctttggggattagctccagttcacactgttgcaacctattgttcagtccattgtagcgtgtgtgtagcccaagatataaaggccatgctgacttgacgtcatccccaccttcctccccgtttcgcggggcagtctcgctagaaaatttaactagcaacaagggttgcgctcgttgcaggacttaaccaaacacctcacggcacgagctgacgacagccatgcagcacctgtgcaaactcctgacttaacaggtcgtttccctttcggttcactacttcatgcatgtcaaatcttggtaaggttcttcgtgtagcatcgaattaaaccacacgctccgctgcttgtgcgggcccccgtcaattcctttgagttttagccttgcgaccgtactccccaggcgggacacttaaagcgttagcttcggcacagagagggtcgatactccctatacctagtgtccatagtttaaggcgtggactaccagagtatctaattctgttcgctccccacgctttcgagcctcagtgtcagtaacaacctagaaaaccgccttcgcctctggtgttcctcccgatatctacgcattttaccactacaccgggaattccgttttctcctgctgtactctagtccaacagtattgaatgacacgtcccggttaagccgggaaatttcacatccaacttgaaagaccacctacgctcactttacgcccaataaatccggataacgcttgcttcctacgtattaccgcggctgctggcacgtagttagccgaagcttattccacaggtaccgtcattattcttccctgtgaaaagaggtttacaacccgaaaaccttcttcactcacgcggtattgctgcgtcagggttgcccccattgcgcaatattccccactgctgcctcccgtaggagtctggaccgtgtctcagttccagtgtgaccgttcgccctctcagaccggttacccatcgtcgccttggtgggctgttatctcaccaactagctaatgggacgcgggtccatcctatggcaccggagcgtttaatacaaaagccatgcgactctcatatattataaggcattactcccagtttcccgaggctattcctttccatagggcaggttacccacgcgttactcacccgttcgccactttctaggatttcattcccccgaangggtcattccatcccgtctcgttcgacttgcatgtgttaagcataccgccagcgttcgtcctgagccaggatcaaactcta

SEQ ID NO: 5 - DNA, 1516 bp, Artificial Sequence
  Uncultured Bacteria
tttgatcctggctcaggatgaacgctggcggcgtgcctaatacatgcaagtcgaacgaagagaggagcttgctcctctcttagtggcgaacgggtgagtaatacataagcaacctacccatacgaccgggataacagttggaaacgactgctaataccggataggtgaccgggggacatcccctgatcattaaagctgggacacagcacgaatggatgggcttatggcgcattagttarttggcggggtaacggcccaccaagacgatgatgcgtagccgacctgagagggtgaccggccacactgggactgagacacggcccagactcctacgggaggcagcagtagggaattttcggcaatgggcgcaagcctgaccgagcaacgccgcgtgagtgaagaaggccttcgggttgtaaagctctgttgtaagggaagaatggccaagagagggaatgctcttggagtgacggtaccttaccagaaagccacggttaactacgtgccagcagccgcggtaatacgtaggtggcaagcgttatccggaattattgggcgtaaagggagcgtaggcggtttgataagtctgaggtgaaagttcggggctcaactccggattgccttagaaactgtcagactagagtgcaggagagggcaatggaattccatgtgtagcggtaaaatgcgtagatatatggaggaacaccagtggcgaaggcggttgcctggcctgtaactgacgctgaggctcgaaagcgtggggagcaaataggattagataccctagtagtccacgctgtaaacgatgattactaagtgttggagcaattcagtgctgcagttaacgcaataagtaatccgcctggggagtatgcacgcaagtgtgaaactcaaaggaattgacgggggcccgcacaagcggtggagtatgtggtttaattcgaagcaacgcgaagaaccttaccaagtcttgacataccgcgcaaggccctggagacagggagatagttatggcggatacaggtggtgcatggttgtcgtcagctcgtgtcgtgagatgttgggttaagtcccgcaacgagcgcaacccttatctttagttaccatcattcagttggggactctagagagactgccggtgacaaaccggaggaaggtggggatgacgtcaaatcatcatgccccttatgacttgggctacacacgtactacaatggcgtatacagagagacgcgaaatggcgacatggagcaaacctcacaaagtacgtctcagttcagattgaagtctgcaactcgacttcatgaaggcggaatcgctagtaatcgcggatcagcatgccgcggtgaatacgttctcgggccttgtacacaccgcccgtcaaaccatgagagttggtaatacccgaagccggtggcctaacccagtaatgggagggagccgtcgaaggtaggatcgatgactggggttaagtcgtaacaaggtatccctacgggaacgtgcggctggatcacct

SEQ ID NO: 6 - DNA, 1569 bp, Acetobacterium
tagtcctgcaggtttaaacgaattcgcccttaaggaggtgatccagccgcaccttccgatacggctaccttgttacgacttcaccccaattactgaccccaccttcggcagctggctccttgcggttgcctcactgacttcgggtgttgccaactctcgtggtgtgacgggcggtgtgtacaagacccgggaacgcattcaccgcagcattctgatctgcgattactagcaactccaacttcatgcaggcgagttgcagcctgcaatccgaactgagatctgttttaagggattagcttcaccccgcggtttcgcagccctctgttcagaccattgtagcacgtgtgtagcccaggtcataaggggcatgatgatttgacgtcgtccccaccttcctccgtgttatccacggcagtctgcttagagtgcccaactaaatgatggcaactaaccacaggggttgcgctcgttgcgggacttaacccaacatctcacgacacgagctgacgacaaccatgcaccacctgtctctctgtccccgaagggaaagcccaatctcttgggttgtcagaggatgtcaagacctggtaaggttcttcgcgttgcttcgaattaaaccacatgctccgctgcttgtgcgggtccccgtcaattcctttgagtttcaaccttgcggtcgtactccccaggcggagtgcttattgcgttagctgcggcactgagtctccccaacacctagcactcatcgtttacggcgtggactaccagggtatctaatcctgtttgctccccacgctttcgcacctcagcgtcagtatttgtccagcaagccgccttcgccaccggtgttcctcctaatatctacgcatttcaccgctacactaggaattccacttgcctctccaatactcaagtctttcagtttcaaatgcatgtcaccggttgagccggtacctttcacatctgacttaaaaaaccgcctgcgtgccctttacgcccagtaaatccggacaacgcttgtcccctacgtattaccgcggctgctggcacgtagttagccgggactttcttcttgggtaccgtcttttttcttccccaataacagagctttacgatccgaaaaccttcttcactcacgcggtattgctgcgtcagggttgcccccattgcgcaatattccccactgctgcctcccgtaggagtctggaccgtgtctcagttccagtgtgaccgttcgccctctcagaccggttacccatcgttgccttggtgggctgttatctcaccaactagctaatgggacgcgggtccatcctatggcaccggagcgtttaatacaaaagccatgcgactctcgtgtattataaggcattactcccagtttcccgaggctattcctttccatagggcaggttacccacgcgttactcacccgttcgccactttctaggatttcattcccccgaagggtcattccatcccgtctcgttcgacttgcatgtgttaagcataccgccagcgttcgtcctgagccaggatcaaactctaagggcgaattcgcggccgcta

SEQ ID NO: 7 - DNA, 1528 bp, Artificial Sequence
  Uncultured Bacteria
gagtttgatcctggctcagaacaaacgctggcggcgtgtcttaagcatgcaagtcgagcggcaggcgcagcaatgcgctgagagcggcggactggtgagtaacgcgtgggtaatctacctttggcatggggatagccactagaaatagtgggtaataccgaatacgttccctggggggaggtttcagggaagaaagggtgctacggcaccggccaaagatgagctcgcgtcccattagctagttggtgaggtaatggcccaccaaggcgatgatgggtagccggcctgagagggtgtacggccacactgggactgagatacggcccagacccctacgggaggcagcagctaagaatattccgcaatggacgaaagtctgacggagcgacgccgcgtggatgatgaaggccgaaaggttgtaaagtccttttgctggggaagaataagtgtgggagggaatgcccgcatgatgacatgaaccggcgaataagccccggccaactacgtgccagcagccgcggtaacacgtagggggcgagcgttgttcggaattactgggcgtaaagggcatgtaggcggtctggtaagcctggcgtgaaaggccacagctcaactgtgggattgcgttgggaactgctgggcttgagttatggagagggagctagaattcctggtgtaggggtgaaatctgtagatatcagaagaataccgatggcgaaggcaagctcctggccgatgactgacgctgaggtgcgaaagtgtggggatcaaacaggattagataccctggtagtccacactgtaaacgatgtgcactaggtgctggggcggtagcttcagtgccggagcgaacgtggtaagtgcaccgcctggggagtatgctcgcaagggtgaaactcaaaggaattgacgggggcccgcacaagcggtggagcatgtggtttaattcgatggtacgcgaggaaccttacctgggtttgacatacagggggatgtggcagcgatgtcacagactggaaacagtaccctgtacaggtgctgcatggctgtcgtcagctcgtgccgtgaggtgttgggttaagtcccgcaacgagcgcaacccctactgccagttactaacaggtaacgctgaggactctggcggaactgccggtgacaaaccggaggaaggtggggatgacgtcaagtcatcatggcccctatgtccagggctacacacgtgctacaatgggcgatacagagtgacgcgaagccgcgaggtggagcaaatcgcagaaaatcgctctcagttcggattggagtctgaaacccgactccatgaaggtggaatcgctagtaatcgcgcatcagcatggcgcggtgaatacgttcccgggccttgtacacaccgcccgtcacaccatccgagtggggggtacccgaagccgttagtccaacctgcaaaggggggcgacgtcgaaggtacgtcttgtgaggagggtgaagtcgtaacaaggtagtcgtaccggaaggtgcggctggatcacct

SEQ ID NO: 8 - DNA, 845 bp, Acetobacterium
ttattgcgttagctgcggcactgagtctccccaacacctakcactcatcgtttacggcgtggactaccagggtatctaatcctgtttgctccccacgctttcgcacctcagcgtcagtatttgtccagcaagccgccttcgccaccggtgttcctcctaatatctacgcatttcaccgctacactaggaattccacttgcctctccaatactcaagtctttcagtttcaaatgcatgtcaccggttgagccggtacctttcacatctgacttaaaaaaccgcctgcgtgccctttacgcccagtaaatccggacaacgcttgtcccctacgtattaccgcggctgctggcacgtagttagccgggactttcttcttgggtaccgtcttttttcttccccaataacagagctttacgatccgaaaaccttcttcactcacgcggtattgctgcgtcagggttgcccccattgcgcaatattccccactgctgcctcccgtaggagtctggaccgtgtctcagttccagtgcgaccgttcgccctctcagaccggttacccatcgttgccttggtgggctgttatctcaccaactagctaatgggacgcgggtccatcctatggcaccggagcgtttaatacaaaagccatgcgactytcgtatattataaggcattactcccagtttcccgaggctattcctttccatagggcaggttacccacgcgttactcacccgttcgccactttctaggatttcattcccccgaagggtcattccatcccgtctcgtttgacttgcatgtgttaagcataccgccagcgttcgtcctgagccaggatcaaa

SEQ ID NO: 9 - DNA, 1484 bp, Dehalococcoides sp.
aggtgatccagccgcagcttccgctacggctaccttgttacgacttcgtcccaattaccagtcccaccctcggcgactgcctccttgcggttggcacatcgacttcaagtgttaccggctttcatgacgtgacgggcggtgtgtacaaggcccgagaacgtattcaccgcaccttgctgatatgcggttactagcaactccaacttcatgcaggcgggtttcagcctgcaatccgaactgaggacagctttggggattagctccagttcacactgttgcaacctattgttctgtccattgtagcgtgtgtgtagcccaagatataaaggccatgctgacttgacgtcatccccaccttcctccccgtttcgcggggcagtctcgctagaaaatttaactagcaacaagggttgcgctcgttgcaggacttaaccaaacacctcacggcacgagctgacgacagccatgcagcacctgtgcaaactcctgacttaacaggtcgtttccctttcggttcactacttcatgcatgtcaaatcttggtaaggttcttcgtgtagcatcgaattaaaccacacgctccgctgcttgtgcgggcccccgtcaattcctttgagttttagccttgcgaccgtactccccaggcgggacacttaaagcgttagcttcggcacagagagggtcgatactccctatacctagtgtccatagtttaaggcgtggactaccagagtatctaattctgttcgctccccacgctttcgagcctcagtgtcagtgacaacctagaaaaccgccttcgcctctggtgttcctcccgatatctacgcattttaccactacaccgggaattccgttttctcctgctgtactctagtccaacagtattgaatgacacgtcccggttaagccgggaaatttcacatccaacttgaaagaccacctacgctcactttacgcccaataaatccggataacgcttgcttcctacgtattaccgcggctgctggcacgtagttagccgaagcttattccacaggtaccgtcattattcttccctgtgaaaagaggtttacaacccgaaagccttcatccctcacgcggcgttgctgggtcaggctttcgcccattgcccaagattccttgctgctgcctcccgtaggagtctgggccgtgtctcagtcccagtgtggctgaacatcctctcagaccagctaccgatcgaagccttggtaggccattaccccaccaactagctaatcggacgcaagcccctcaccaagcaccttgcggctttaatgaaccgacttatgtcagcccatcacatgcggtattaccttcagtttcccgaagctatcccccacttagaggtaggttacttacgcgttactcacccgtttgccactatcttaattgcttaagaccgttcgacttgcatgcataaggcacgccgctagcgttcatcctgagccaggatcaaactc

SEQ ID NO: 10 - DNA, 1520 bp, Clostridium
aggtgatccagccgcaggttctcctacggctaccttgttacgacttcaccccaatcaccaatcccaccttcggccgctggctccttacggttacctcacggacttcgggtgttaccggctctcatggtgtgacgggcggtgtgtacaagacccgggaacgtattcaccgcgacatgctgattcgcgattactagcaactccaacttcatgcaggcgagtttcagcctgcaatccgaactgggaccggcttttgagtttggctccccctcgcgggtttgctgcttgttgtaccggccattgtagcacgtgtgtagccctagacataaggggcatgatgatttgacgtcatccccaccttcctcctggttaacccaggcagtctcgttagagtgctcaacctaatggtagcaactaacgataagggttgcgctcgttgcgggacttaacccaacatctcacgacacgagctgacgacaaccatgcaccacctgtcttcctgtccccgaagggacttccccgattaagggtaattcaggagatgtcaagtctaggtaaggttcttcgcgttgcttcgaattaaaccacatgctccgctgcttgtgcgggtccccgtcaattcctttgagttttaatcttgcgaccgtactccccaggcggaatacttattgcgtttgctgcggcaccgagggtggtaccccccgacacctagtattcatcgtttacggcgtggactaccagggtatctaatcctgtttgctccccacgctttcgtatctcagcgtcagttacggtccagaaagtcgccttcgccactggtgttcttcctaatctctacgcatttcaccgctacactaggaattccgctttcctctccagcactcaagaccatcagtttcaaatgcagcgcccgagttaagcccgggaatttcacatctgacttgacagcccgcctacacgccctttacacccagtaattccggacaacgctcgctccctacgtattaccgcggctgctggcacgtagttagccggagcttgttttcgaggtaccgtcatttgtttcgtccctcgtcaaaaaagtttacaatccgaagaccttcttccttcacgcggcgttgctgcgtcagggttgcccccattgcgcaatattccccactgctgcctcccgtaggagtctggaccgtgtctcagttccagtgtgaccgttcgccctctcagaccggttacccatcgtcgccttggtgggctgttatctcaccaactagctaatgggacgcgggtccatcctatggcaccggagcgtttaatacaaaagccatgcgactctcatatattataaggcattactcccagtttcccgaggctattcctttccatagggcaggttacccacgcgttactcacccgttcgccactttctaggatttcattcccccgaagggtcattccatcccgtctcgttcgacttgcatgtgttaagcataccgccagcgttcgtcctgagccaggatcaaactcta

SEQ ID NO: 11 - DNA, 1484 bp, Dehalococcoides sp.
gagtttgatcctggctcaggacgaacgctagcggcgtgccttatgcatgcaagtcgaacggtcttaagcaattaagatagtggcaaacgggtgagtaacgcgtaagtaacctacctctaagtgggggatagcttcgggaaactgaaggtaataccgcatgtgatgggctgacataagtcggttcattaaagccgcaaggtgcttggtgaggggcttgcgtccgattagctagttggtggggtaatggcctaccaaggcttcgatcggtagctggtctgagaggatgatcagccacactgggactgagacacggcccagactcctacgggaggcagcagcaaggaatcttgggcaatgggcgaaagcctgacccagcaacgccgcgtgagggatgaaggctttcgggttgtaaacctcttttcacagggaagaataatgacggtacctgtggaataagcttcggctaactacgtgccagcagccgcggtaatacgtaggaagcaagcgttatccggatttattgggcgtaaagtgagcgtaggtggtctttcaagttggatgtgaaatttcccggcttaaccgggacgtgtcattcaatactgttggactagagtacagcaggagaaaacggaattcccggtgtagtggtaaaatgcgtagatatcgggaggaacaccagaggcgaaggcggttttctaggttgtcactgacactgaggctcgaaagcgtggggagcgaacagaattagatactctggtagtccacgccttaaactatggacactaggtatagggagtatcgaccctctctgtgccgaagctaacgctttaagtgtcccgcctggggagtacggtcgcaaggctaaaactcaaaggaattgacgggggcccgcacaagcagcggagcatgtggtttaattcgatgctacacgaagaaccttaccaagatttgacatgcatgaagtagtgaaccgaaagggaaacgacctgttaagtcaggagtttgcacaggtgctgcatggctgtcgtcagctcgtgccgtgaggtgtttggttaagtcctgcaacgagcgcaacccttgttgctagttaaattttctagcgagactgccccgcgaaacggggaggaaggtggggatgacgtcaagtcagcatggcctttatatcttgggctacacacacgctacaatggacagaacaataggttgcaacagtgtgaactggagctaatccccaaagctgtcctcagttcggattgcaggctgaaacccgcctgcatgaagttggagttgctagtaaccgcatatcagcaaggtgcggtgaatacgttctcgggccttgtacacaccgcccgtcacgtcatgaaagccggtaacacttgaagtcgatgtgccaaccgcaaggaggcagtcgccgagggtgggactggtaattgggacgaagtcgtaacaaggtagccgtagcggaagctgcggctggatcacct

SEQ ID NO: 12 - DNA, 1507 bp, Artificial Sequence
  Uncultured Bacteria
gagtttgatcctggctcaggacgaacgctggcggcgtgcttaacacatgcaagtcgaacggtcagcaccgggaggttcgctggaaggtgctggtagtggcggacgggtgagtaacgcgtgaggacatggctccaggagggggataacgtttggaaacggacgctaagaccccatatgccggaaggtgaaactgggcaaccagcctggagagtgactcgcgtcctatcagctagttggtggggtgaaggcctaccaaggcgatgacgggtagccgacctgagagggtggccggccacactggaactgagatacggtccagactcctacgggaggcagcagtggggaatattgggcaatgggcgaaagcctgacccagcgacgccgcgtgggggaagaagtctttcgggatgtaaacccttgttgtacgggacgaaggaagtgacggtaccgtacgaggaagccccggcaaactacgtgccagcagccgcggtaatacgtagggggcgagcgttgtccggaattactgggcgtaaagcgcacgtaggcgggctgttaagtcggccgtgaaaggcactggctcaaccggtgtttgtcggtcgatactggcagtctggagtatgggagagggaactggaattcccggtgtagcggtgaatgcgtagatatcgggaggaacaccagtggcgaaggcgggttcctggcccattactgacgctgaggtgcgaaagccgggggagcgaacgggattagataccccggtagtcccggctgtaaacgatggatgctaggtgtgggggggttcccctctgtgccgcagctaacgcgataagcatcccgcctggggagtacgatcgcaagattgaaactcaaaggaattgacgggggcccgcacaagcggtggagcacgtggtttaattcgatgcaaaccgaagaaccttacctgggtttgacatgcacgtggtacggagatgaaagtcgaaggaccctgtagaaatacagggagcgtgcacaggtgctgcatggctgtcgtcagctcgtgtcgtgaggtgttgggttaagtcccgcaacgagcgcaacccctgcatccagttgccagcgagtcaagtcggggactctggatggactgccggcgacaagccggaggaaggtggggatgacgtcaagtcatcatggcccttatgtccagggcgacacacgtgctacaatggccggcacaacgggaagcgaaggggcgacccggagcggatccaatcaaagccggtcccagttcggattggagtctgcaacccgactccatgaagctggaatcgctagtaatcgcggatcagccaagccgcggtgaatacgttcccgggccttgtacacaccgcccgtcacaccacccgagttgggggcacccgaagccggaggcttaacccgtaaggggaagatccgtcgaaggtgcgtctggtaaggggggtgaagtcgtaacaaggtagccgtaccggaaggtgcggctggatcacct

SEQ ID NO: 13 - DNA, 1484 bp, Dehalococcoides sp.
aggtgatccagccgcagcttccgctacggctaccttgttacgacttcgtcccaattaccagtcccaccctcggcgactgcctccttgcggttggcacatcgacttcaagtgttaccggctttcatgacgtgacgggcggtgtgtacaaggcccgagaacgtattcaccgcaccttgctgatatgcggttactagcaactccaacttcatgcaggcgggtttcagcctgcaatccgaactgaggacagctttggggattagctccagttcacactgttgcaacctattgttctgtccattgtagcgtgtgtgtagcccaagatataaaggccatgctgacttgacgtcatccccaccttcctccccgtttcgcggggcagtctcgctagaaaatttaactaacaacaagggttgcgctcgttgcaggacttaaccaaacacctcacggcacgagctgacgacagccatgcagcacctgtgcaaactcctgacttaacaggtcgtttccctttcggttcactacttcatgcatgtcaaatcttggtaaggttcttcgtgtagcatcgaattaaaccacacgctccgctgcttgtgcgggcccccgtcaattcctttgagttttagccttgcgaccgtactccccaggcgggacacttaaagcgttagcttcggcacagagagggtcgatactccctatacctagtgtccatagtttaaggcgtggactaccagagtatctaattctgttcgctccccacgctttcgagcctcagtgtcagtgacaacctagaaaaccgccttcgcctctggtgttcctcccgatatccacgcattttaccactacaccgggaattccgttttctcctgctgtactctagtccaacagtattgaatgacacgtcccggttaagccgggaaatttcacatccaacttgaaagaccacctacgctcactttacgcccaataaatccggataacgcttgcttcctacgtattaccgcggctgctggcacgtagttagccgaagcttattccacaggtaccgtcattattcttccctgtgaaaagaggtttacaacccgaaagccttcatccctcacgcggcgttgctgggtcaggctttcgcccattgcccaagattccttgctgctgcctcccgtaggagtctgggccgtgtctcagtcccagtgtggctgatcatcctctcagaccagctaccgatcgaagccttggtaggccattaccccaccaactagctaatcggacgcaagcccctcaccaagcaccttgcggctttaatgaaccgacttatgtcagcccatcacatgcggtattaccttcagtttcccgaagctatcccccacttagaggtaggttacttacgcgttactcacccgtttgccactatcttaattgcttaagaccgttcgacttgcatgcataaggcacgccgctagcgttcatcctgagccaggatcaaactc

SEQ ID NO: 14 - DNA, 1511 bp, Acetobacterium
gagtttgatcctggctcaggacgaacgctggcggtatgcttaacacatgcaagtcgaacgagacgggatggaatgacccttcgggggaatgaaatcctagaaagtggcgaacgggtgagtaacgcgtgggtaacctgccctatggaaaggaatagcctcgggaaactgggagtaatgccttatagtatacagaagtcgcatggcttttgtattaaacgccccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaaaagacggtacccaagaagaaagtcccggctaactacgtgccagcagccgcggtaatacgtaggggacaagcgttgtccggatttactgggcgtaaagggcacgcaggcggttttttaagtcagatgtgaaaggtaccggctcaaccggtgacatgcatttgaaactgaaagacttgagtattggagaggcaagtggaattcctagtgtagcggtgaaatgcgtagatattaggaggaacaccggtggcgaaggcggcttgctggacaaatactgacgctgaggtgcgaaagcgtggggagcaaacaggattagataccctggtagtccacgccgtaaacgatgagtgctaggtgttggggagactcagtgccgcagctaacgcaataagcactccgcctggggagtacgaccgcaaggttgaaactcaaaggaattgacggggacccgcacaagcagcggagcatgtggtttaattcgaagcaacgcgaagaaccttaccaggtcttgacatcctctgacaacccaagagattgggctttcccttcggggacagagagacaggtggtgcatggttgtcgtcagctcgtgtcgtgagatgttgggttaagtcccgcaacgagcgcaacccctgtggttagttgccatcatttagttgggcactctaagcagactgccgtggataacacggaggaaggtggggacgacgtcaaatcatcatgccccttatgacctgggctacacacgtgctacaatggtctgaacagagggctgcgaaaccgcgaggtgaagctaatcccttaaaacagatctcagttcggactgcaggctgcaactcgcctgcatgaagttggagttgctagtaatcgcagatcagaatgctgcggtgaatgcgttcccgggtcttgtacacaccgcccgtcacaccacgagagtcggcaacacccgaagtcagtgaggcaaccgcaaggagccagctgccgaaggtggggtcagtaattggggtgaagtcgtaacaaggtagccgtatcggaaggtgcggctggatcacct

SEQ ID NO: 15 - DNA, 1507 bp, Artificial Sequence
  Uncultured Bacteria
aggtgatccagccgcaccttccggtacggctaccttgttacgacttcaccccccttaccagacgcaccttcgacggatcttccccttacgggttaagcctccggcttcgggtgcccccaactcgggtggtgtgacgggcggtgtgtacaaggcccgggaacgtattcaccgcggcttggctgatccgcgattactagcgattccagcttcatggagtcgggttgcagactccaatccgaactgggaccggctttgattggatccgctccgggtcgccccttcgcttcccgttgtgccggccattgtagcacgtgtgtcgccctggacataagggccatgatgacttgacgtcatccccaccttcctccggcttgtcgccggcagtccatccagagtccccgacttgactcgctggcaactggctgcaggggttgcgctcgttgcgggacttaacccaacatctcacgacacgagctgacgacagccatgcagcacctgtgcacgctccctgtatttctacagggtccttcgactttcatctccgtaccacgtgcatgtcaaacccaggtaaggttcttcggtttgcatcgaattaaaccacgtgctccaccgcttgtgcgggcccccgtcaattcctttgagtttcaatcttgcgatcgtactccccaggcgggatgcttatcgcgttagctgcggcacagaggggaacccccccacacctagcatccatcgtttacagccgggactaccggggtatctaatcccgttcgctcccccggctttcgcacctcagcgtcagtaatgggccaggaacccgcctcgccactggtgttcctcccgatatctacgcatttcaccgctacaccgggaattccagttccctctcccatactccagactgccagtatcgaccgacaaacaccggttgagccagtgcctttcacggccgacttaacagcccgcctacgtgcgctttacgcccagtaattccggacaacgctcgccccctacgtattaccgcggctgctggcacgtagtttgccggggcttcctcgtacggtaccgtcacttccttcgtcccgtacaacaagggtttacatcccgaaagacttcttcccccacgcggcgtcgctgggtcaggctttcgcccattgcccaatattccccactgctgcctcccgtaggagtctggaccgtatctcagttccagtgtggccggccaccctctcaggtcggctacccgtcatcgccttggtaggccttcaccccaccaactagctgataggacgcgagtcactctccaggctggttgcccagtttcaccttccggcatatggggtcttagcgtccgtttccaaacgttatccccctcctggagccatgtcctcacgcgttactcacccgtccgccactaccagcaccttccagcgaaccttccggtgctgaccgttcgacttgcatgtgttaagcacgccgccagcgttcgtcctgagccaggatcaaactc

SEQ ID NO: 16 - DNA, 1513 bp, Acetobacterium
aggtgatccagccgcaccttccgatacggctaccttgttacgacttcaccccaattactgaccccaccttcggcagctggctccttgcggttgcctcactgacttcgggtgttgccaactctcgtggtgtgacgggcggtgtgtacaagacccgggaacgcattcaccgcagcattctgatctgcgattactagcaactccaacttcatgcaggcgagttgcagcctgcaatccgaactgagatctgttttaagggattagcttcacctcgcggtttcgcagccctctgttcagaccattgtagcacgtgtgtagcccaggtcataaggggcatgatgatttgacgtcgtccccaccttcctccgtgttatccacggcagtctgcttagagtgcccaactaaatgatggcaactaaccacaggggttgcgctcgttgcgggacttaacccaacatctcacgacacgagctgacgacaaccatgcaccacctgtctctctgtccccgaagggaaagcccaatctcttgggttgtcagaggatgtcaagacctggtaaggttcttcgcgttgcttcgaattaaaccacatgctccgctgcttgtgcgggtccccgtcaattcctttgagtttcaaccttgcggtcgtactccccaggcggagtgcttattgcgttagctgcggcactgagtctccccaacacctagcactcatcgtttacggcgtggactaccagggtatctaatcctgtttgctccccacgctttcgcacctcagcgtcagtatttgtccagcaagccgccttcgccaccggtgttcctcctaatatctacgcatttcaccgctacactaggaattccacttgcctctccaatactcaagtctttcagtttcaaatgcatgtcaccggttgagccggtacctttcacatctgacttaaaaaaccgcctgcgtgccctttacgcccagtaaatccggacaacgcttgtcccctacgtattaccgcggctgctggcacgtagttagccgggactttcttcttgggtaccgtcttttttcttccccaataacagagctttacgatccgaaaaccttcttcactcacgcggtattgctgcgtcagggttgcccccattgcgcaatattccccactgctgcctcccgtaggagtctggaccgtgtctcagttccagtgtgaccgttcgccctctcagaccggttacccatcgttgccttggtgggctgttatctcaccaactagctaatgggacgcgggtccatcctatggcactggagcgtttaatacaaaagccatgcgactctcgtatattataaggcattactcccagtttcccgaggctattcctttccatagggcaggttacccacgcgttactcacccgttcgccactttctaggatttcattcccccgaagggtcattccatcccgtctcgttcgacttgcatgtgttaagcataccgctagcgttcgtcctgagccaggatcaaactcta

SEQ ID NO: 17 - DNA, 1496 bp, Clostridium
gagtttgatcctggctcaggacgaacgctggcggcgtgcctaacacatgcaagtcgagcgatgaagctccttcgggagtggattagcggcggacgggtgagtaacacgtgggcaacctgcctcaaagtggggaatagccctccgaaaggaggattaataccgcataacatgaaggagtcgcatggtaccttcattaaagatttatcgctttgagatgggcccgcggcgcattagctagttggtgaggtaacggctcaccaaggcgacgatgcgtagccgacctgagagggtgatcggccacattggaactgagacacggtccagactcctacgggaggcagcagtggggaatattgcgcaatgggggaaaccctgacgcagcaacgccgcgtgagtgatgaaggtcttcggattgtaaaactctgtctttggggacgataatgacggtacccaaggaggaagccacggctaactacgtgccagcagccgcggtaatacgaaggtggcaagcgttgtccggatttactgggcgtaaagagtatgtaggcggatatttaagtcagatgtgaaattcccgggcttaacctgggagctgcatttgatactgggtatctagagtgcgggagaggaaagtggaattcctagtgtagcggtgaaatgcgtagagattaggaagaacaccagtggcgaaggcgactttctggaccgtaactgacgctgagatacgaaagcgtggggagcaaacaggattagataccctggtagtccacgccgtaaacgatgaatactaggtgtcggggggtaccaccctcggtgccgcagcaaacgcaataagtattccgcctggggagtacggtcgcaagattaaaactcaaaggaattgacggggacccgcacaagcagcggagcatgtggtttaattcgaagcaacgcgaagaaccttacctagactcgacatctcctgaattacccttaatcggggaagtcccttcggggacaggaagacaggtggtgcatggttgtcgtcagctcgtgtcgtgagatgttgggttaagtcccgcaacgagcgcaacccttatcgttagttgctaccattaggttgagcactctagcgagactgcctgggttaaccaggaggaaggtggggatgacgtcaaatcatcatgccccttatgtctagggctacacacgtgctacaatggccggtacaacaagcagcaaacccgcgagggggagccaaactcaaaagccggtcccagttcggattgcaggctgaaactcgcctgcatgaagttggagttgctagtaatcgcgaatcagcatgtcgcggtgaatacgttcccgggtcttgtacacaccgcccgtcacaccatgagagccggtaacacccgaagtccgtgaggtaaccgtaaggagccagcggccgaaggtgggattggtgattggggtgaagtcgtaacaaggtagccgtaggagaacctgcggctggatcacct

SEQ ID NO: 18 - DNA, 1010 bp, Dehalococcoides sp.
aggtgatccagccgcaaacccggtgggatacattgtcaaaagaaatgccggctgtttcaacaaccggcaataccctcagaggcggtctccgctgaagtgcccgtatccgggcaaacagttcatcacaggcaaatggcttgcaaagatagtcatctgcaccggcatccaaaccctttacctttgaatcaagcgaatctctggcggtaagcatgagcacaggtgtaactatgccgtcagccctgagcttccggcagacctcaatatcatttttatccggcaagccgatatccagaatcagcaggtcaaattcagaatttgcggcaaagtattcccccgaagccccgtcacaggcatattcaaccacatgcccggcctcaccaagagcccgctccagggtttggcagagacacttatcatcatcaagtaataaaatccgcacgccgcaaagccttttacctgttaatcattcataattcctataatagcactattttggcctcagcggtcatccaataatcatcttttggtcatttattgttcatctttatatgctaggcttgtactaattagctatgaggagtaattatgattagagtattagcctcggtattactcagtgtcatggtatccgccctgccggttgcagccaatgtatcagcggcagaaatagcaagcaagtattatccgaatccccaggtctttgacgacacttatggcactatcctcagcccttcggaaatagtggccctgtaccctatgtccgcagaagatgaagccaagcttattcaggttataccccaatgcccggtaaatgtagacggagtgtggtataaagcagaggaaatcacccttttcaacgggcatcagcttcactttactacagataaaaaaggcgggttatacgcttttactgatgccagagccatggaaatattccttgaagcagaatacggggatatattcaatacttccactgacaaagcgatgcagatgctgcggctggatcacct

SEQ ID NO: 19 - DNA, 1531 bp, Dehalococcoides sp.
aggtgatccagccgcaggttctcctacggctaccttgttacgacttcaccccaatcaccagtcccaccctcggcgactgcctccttgcggttggcacatcgacttcaagtgttaccggctttcatgacgtgacgggcggtgtgtacaaggcccgagaacgtattcaccgcaccttgctgatatgcggttactagcaactccaacttcatgcaggcgggtttcagcctgcaatccgaactgaggacagctttggggattagctccagttcacactgttgcaacctattgttctgtccattgtagcgtgtgtgtagcccaagatataaaggccatgctgacttgacgtcatccccaccttcctccccgtttcgcggggcagtctcgctagaaaatttaactagcaacaagggttgcgctcgttgcaggacttaaccaaacacctcacggcacgagctgacgacagccatgcagcacctgtgcaaactcctgacttaacaggtcgtttccctttcggttcactacttcatgcatgtcaaatcttggtaaggttcttcgtgtagcatcgaattaaaccacacgctccgctgcttgtgcgggcccccgtcaattcctttgagttttagccttgcgaccgtactccccaggcgggacacttaaagcgttagcttcggcacagagagggtcgatactccctatacctagtgtccatagtttaaggcgtggactaccagagtatctaattctgttcgctccccacgctttcgagcctcagtgtcagtgacaacctagaaaaccgccttcgcctctggtgttcctcccgatatctacgcattttaccactacaccgggaattccgttttctcctgctgtactctagtccaacagtattgaatgacacgtcccggttaagccgggaaatttcacatccaacttgaaagaccacctacgctcactttacgcccaataaatccggataacgcttgcttcctatgtattaccgcggctgctggcacgtagttagccggtgcttccttcagaggtaccgtcaagaaaaccgggtattaaccgactatcatttcttccctctagacagagctttacgacccgaaagccttcatcactcacgcggcgttgctgcgtcagggtttcccccattgcgcaaaattccccactgctgcctcccgtaggagtctggaccgtgtctcagttccagtgtggctgatcatcctctcagaccagctaaccatcgtcgccttggtaggctcttaccctaccaactagctaatggtacgcagactcatcttgaatcaggagcatattcagaggcccccttttcccgcaatgacttacgccatcgtgggcttatccggtattagcacccctttcgagatgttatcccagagtccaaggcagattatctacgcgttactcacccgtgcgccactaaatgaaagagcaagctccttcactccgttcgacttgcatgtgttaggcacgccgccagcgttcgttctgagccaggatcaaactc

SEQ ID NO: 20 - DNA, 1510 bp, Acetobacterium
aggtgatccagccgcaccttccgatacggctaccttgttacgacttcaccccaattactgaccccaccttcggcagctggctccttgcggttgcctcactgacttcgggtgttgccaactctcgtggtgtgacgggcggtgtgtacaagacccgggaacgcattcaccgcagcattctgatctgcgattactagcaactccaacttcatgcaggcgagttgcagcctacaatccgaactgagatctgttttaagggattagcttcacctcgcggtttcgcagccctctgttcagaccattgtagcacgtgtgtagcccaggtcataaggggcatgatgatttgacgtcgtccccaccttcctccgtgttatccacggcagtctgcttagagtgcccaactaaatgatggcaactaaccacaggggttgcgctcgttgcgggacttaacccaacatctcacgacacgagctgacgacaaccatgcaccacctgtctctctgtccccgaagggaaagcccaatctcttgggttgtcagaggatgtcaagacctggtaaggttcttcgcgttgcttcgaattaaaccacatgctccgctgcttgtgcgggtccccgtcaattcctttgagtttaaccttgcggtcgtactccccaggcggagtgcttattgcgttagctgcggcactgagtctccccaacacctagcactcatcgtttacggcgtggactaccagggtatctaatcctgtttgctccccacgctttcgcacctcagcgtcagtatttgtccagcaagccgccttcgccaccggtgttcctcctaatatctacgcatttcaccgctacactaggaattccacttgcctctccaatactcaagtctttcagtttcaaatgcatgtcaccggttgagccggtacctttcacatctgacttaaaaaaccgcctgcgtgccctttacgcccagtaaatccggacaacgcttgtcccctacgtattaccgcggctgctggcacgtagttagccgggactttcttcttgggtaccgtcttttttcttccccaataacagagctttacgatccgaaaaccttcttcactcacgcggtattgctgcgtcagggttgcccccattgcgcaatattccccactgctgcctcccgtaggagtctggaccgtgtctcagttccagtgtgaccgttcgccctctcagaccggttacccatcgttgccttggtgggctgttatctcaccaactagctaatgggacgcgggtccatcctatggcaccggagcgtttaatacaaaagccatgcgactctcgtatattataaggcattactcccagtttcccgaggctattcctttccatagggcaggttacccacgcgttactcacccgttcgccactttctaggatttcattcccccgaagggtcattccatcccgtctcgttcgacttgcatgtgttaagcataccgccagcgttcgtcctgagccaggatcaaactc

(19) United States
(12) Patent Application Publication
(10) Pub. No.: US 2012/0178147 A1
(43) Pub. Date: Jul. 12, 2012

(54) MICROBIAL CULTURES AND METHODS FOR ANAEROBIC BIOREMEDIATION

(75) Inventors: Rosa Krajmalnik-Brown, Chandler, AZ (US); Rolf U. Halden, Phoenix, AZ (US); Anca G. Delgado, Phoenix, AZ (US); Michal Ziv-El, Tempe, AZ (US)

(73) Assignee: Arizona Board of Regents for and on behalf of Arizona State University, Scottsdale, AZ (US)

(21) Appl. No.: 13/386,386

(22) PCT Filed: Jul. 23, 2010
(86) PCT No.: PCT/US10/43049

§ 371 Date: Mar. 28, 2012

Related U.S. Application Data
(60) Provisional application No. 61/228,059, filed on Jul. 23, 2009.

(51) Int. Cl. C12N 1/20; C02F 3/34; C12S 99/00

(52) U.S. Cl. 435/252.4; 435/262.5; 210/601

(57) ABSTRACT

The invention relates to a consortium of microorganisms that can be used to dehalogenate a chemical composition. Methods of use of the same for biomass production and for use of the same in bioremediation are described.

### RELATED APPLICATIONS

This application claims the benefit of priority of U.S. Provisional Patent Application No. 61/228,059, which was filed on Jul. 23, 2009. The entire text of the aforementioned application is incorporated herein by reference.

### FEDERALLY SPONSORED RESEARCH OR DEVELOPMENT

The research underlying this application was supported, in part, by U.S. Government funds under National Institute of Environmental Health Sciences Grant No. NIEHS 1R01ES015445, and the U.S. Government may therefore have certain rights in the invention.

### FIELD OF THE INVENTION

The present invention relates to anaerobic microbial compositions and methods of use of the same for bioremediation purposes to achieve dechlorination of contaminated samples.

### BACKGROUND OF THE INVENTION

It is now recognized that an effective approach to achieve laboratory scale dechlorination of samples contaminated with chlorinated ethenes would be to inoculate the site with a microbial culture that contains a dechlorinating bacterium. Chloroethenes are among the most common pollutants at hazardous waste sites (ATSDR, 2007), since they have been used extensively as solvents in dry-cleaning operations, metal degreasing, textile finishing, dyeing, and extraction processes in industry. Chlorothenes were carelessly disposed of, handled and stored in the past (Abelson, 1990; McCarty, 1997). Because chlorinated ethenes are highly toxic and known or suspected carcinogens (EPA, 2009), their widespread persistence in the environment poses important health risks and has stimulated investigations concerning their degradation, transport, and fate in the subsurface.

Biodegradation of tetrachloroethene (PCE) and trichloroethene (TCE) is hard to achieve under aerobic conditions; however, anaerobic bioremediation has been proven to be effective for the removal of chloroethenes in groundwater (Aulenta et al., 2006). In order to better understand the reductive dechlorination process of chlorothenes, researchers have been collecting contaminated groundwater and soil samples with the ultimate goal of enriching reductive dechlorinating microorganisms in the laboratory and, in some instances, isolating pure cultures for chloroethenes biodegradation. Thus, anaerobic bacteria that can use chloroethenes as electron acceptors and get energy from dehalorespiration have been isolated and characterized subsequently. Dehalococcoides spp. is a unique group among these bacteria because they are the only ones that can completely dechlorinated PCE or TCE into ethene, an innocuous product (Cupples et al., 2003; He and Löffler, 2003; He et al., 2005; He et al., 2003a; Maymo-Gatell et al., 1997; Muller et al., 2004; Sung et al., 2006b).

Besides groundwater and soil, sediment is also a good source for collecting reductively dechlorinating microorganisms. Inappropriate handling of chloroethenes has led to contamination in rivers, estuaries, and marine environments, and the sediments become a significant temporary chloroethenes sink (Christof et al., 2002; De Rooij et al., 1998; Mazur and Jones, 2001). Furthermore, marine environments provide an important natural source of chloroethenes, since marine organisms like micro- or macro-algae can produce PCE and TCE (Abrahamsson et al., 1995). Additionally, subsurface pyrogenic activity and volcanic eruptions also discharge a lot of chloroethenes to the oceans (Gribble, 1994; Gribble, 2003). The presence of chloroethenes in the sediments may stimulate enzymes for chloroethenes dechlorination by microorganisms and develop beneficial bacteria strains for bioattenuation. However, the reductively dechlorinating bacteria in marine sediments have not been well studied yet (Kittelmann and Friedrich, 2008c), and only a limited number of enriched cultures have been generated from sediments. For example, the bacterium DF-1 was enriched from estuarial sediment (Miller et al., 2005; Wu et al., 2000), and a trans-DCE producing cultures were developed from tidal flat sediment (Kittelmann and Friedrich, 2008c). In these sediment microcosms or cultures, PCE or TCE reductive dechlorination typically produced more trans-DCE than cis-DCE (Griffin et al., 2004; Kittelmann and Friedrich, 2008c; Miller et al., 2005). However, most PCE- or TCE-reductive dechlorinating isolates from groundwater and soil preferentially produce cis-DCE (Cupples et al., 2003; He and Löffler, 2003; He et al., 2005a; He et al., 2003a; Müller et al., 2004; Maymo-Gatell et al., 1997; Sung et al., 2006b). The reductively dechlorinating microbial communities in sediment need to be further studied.

Chlorinated chemicals such as triclocarban (TCC) and triclosan (TCS) also can accumulate in sediment (Audu and Heyn, 1988; Miller et al., 2008; Ying et al., 2007). TCC and TCS are only slightly soluble in water, non-volatile and widely used as antibacterial additives in soaps and other cleaning supplies in the world, with a total amount of disposal in the U.S. environment exceeding 600,000 kg/yr (EPA, 2003). TCC and TCS have antibacterial properties because they can enter cells and block the active site of an enzyme called enoyl-acyl carrier-protein reductase (ENR) (Levy et al., 1999; McMurray et al., 1998). ENR is responsible for producing the fatty acids used in constructing bacteria cell membranes. ENR is absent in humans and other organisms; thus, TCC and TCS are believed to be safe to humans and the environment (Levy et al., 1999; McMurray et al., 1998). However, recent laboratory studies have shown that high concentration of these two biocides may be harmful to organisms, such as algae, fish, bullfrog, rats, rabbits and human beings (Adolfsson-Erici et al., 2002; Boehncke et al., 2003; Chalew and Halden, 2009; Gledhill, 1975; He et al., 2006; Johnson et al., 1963; Nolen and Dierckman, 1979; Orvos et al., 2002; Veldhoen et al., 2006). Moreover, because TCC and TCS can effectively inhibit a wide spectrum of microorganisms, the natural ecological processes conducted by microorganisms in the environment may be disrupted by the persistence of TCC and TCS (Dokianakis et al., 2004; Rosalind A. Neumegen, 2005).

While bioremediation of chlorinated ethene-contaminated sites is a desirable goal, there remains the problem of identifying and making available cultures for the large-scale treatment of chlorinated ethene contamined sites in the field. Further, it is recognized that chlorinated organic compounds such as TCC and TCS inhibit the degradation of chlorinated ethenes because these agents act as bacteriocides against the microbial compositions used to degrade the chlorinated contaminants of soil and water. Thus, there is a significant need to identify cultures for bioremediation of sites with mixtures of these chlorinated contaminants.

### BRIEF SUMMARY OF THE INVENTION

The present invention describes the isolation and the use of a consortium of microorganisms that can be used to dehalogenate a chemical composition. More particularly, it relates to a composition for dehalogenation of a sample that is contaminated with a halogenated chemical comprising a microbial consortium of a mixture of isolated microbial strains of Chloroflexy, Firmicutes and Proteobacteria. In some embodiments, the consortium further comprises one or more microorganisms selected from the group consisting of Spirochaetes, Delta proteobacteria, Beta proteobacteria, Gamaproteobacteria, Acetobacterium, Acidaminobacter, Sedimentibacter, Gracilibacter, and Clostridium. In still other embodiments the consortium comprises at least one strain from microorganisms comprising Trichlorobacter, Geobacter, Chlostridium, and Dehalococcoides.

A particular aspect of the invention relates to a microbial composition for concurrent dehalogenation of a mixture of halogenated ethenes and halogenated antimicrobials, comprising a non-naturally occurring dehalogenating microbial species, wherein said microbial species comprises at least one 16S rDNA nucleic acid sequence that has more than 95% identity to a nucleic acid sequence consisting of SEQ ID NO: 4, a nucleic acid sequence that when translated into protein has more than 80% identity to a nucleic acid sequence consisting of SEQ ID NO: 1, a nucleic acid sequence that when translated into protein has more than 80% identity to a nucleic acid sequence consisting of SEQ ID NO:2, a nucleic acid sequence that when translated into protein has more than 80% identity to a nucleic acid sequence consisting of SEQ ID NO:3, a nucleic acid sequence consisting of SEQ ID NO:4.

For example, the consortium further comprises at least one chloroethene reductase nucleic acid sequence that has more than 80% identity to the group of chloroethene reductases comprised of TceA, BvcA and vcrA.

A preferred aspect of the invention relates to a method for dehalogenating a chemical composition comprising organohalides comprising contacting said chemical composition with the microbial composition of the invention; and concurrently anaerobically dehalogenating said organohalides in said composition. More specifically, the organohalides are halogenated antimicrobial agents. Examples of halogenated antimicrobial agents include at least one of the congeners of triclosan, of triclocarban. Specific examples of organohalide comprises at least one of trichloroethene, cis-1,2-dichloroethene; trans-1,2-dichloroethene, vinyl chloride or tetrachloroethene. These are merely listed as exemplary agents and numerous other halogenated antimicrobial agents are known in the art and may be dehalogenated using these methods.

Also contemplated is a method for dehalogenating halogenated waste, comprising: contacting at least one organohalogen with an isolated bioremediative consortium comprising strains of microorganism comprising Chloroflexy, Spirochaetes, Firmicutes, Proteobacteria; and anaerobically dehalogenating at least one of the congeners of triclosan and triclocarban. In exemplary embodiments, the halogenated waste is taken from the group comprising contaminated soil, contaminated sediment, contaminated water, contaminated industrial wastewater, contaminated domestic wastewater, contaminated sewage sludge, contaminated biosolids.

Also described is a method of producing a microbial dehalogenating consortium comprising culturing microbes of a sediment sample obtained from a site contaminated with a mixture of chlorinated antimicrobials in an anaerobic medium with at least one chlorinated ethane and an electron donor. In specific embodiments, antimicrobial agents may be added to an industrial process stream and the above method used to bioremediate both organohalides present in the process stream and the antimicrobial agent added.

By dehalogenation, or dehalogenating it is intended herein to refer to debromination, deiodination, defluorination or dechlorination of organohalogens. For example, the organohalides may be chlorinated and the methods described herein produce the dechlorination of mono-, di-, tri-, and polychlorinated aliphatics or the dechlorination of mono-, di-, tri-, and polychlorinated aromatics.

In exemplary methods, the methods produce the dehalogenation of mixtures of organohalogens comprising at least two organohalides, comprising fluorinated organics, chlorinated organics, brominated organics, and ionidated organics.

The methods described may also be used for the dehalogenation of organohalogens other than antimicrobials in the presence of antimicrobial agents. Where the methods are for the dehalogenation of antimicrobial agents the antimicrobial agent may be an aromatic antimicrobial agent.

In any of the methods described herein the composition may be formulated as a microbial conglomerate, floc, biofilm pellet or bead. The term “microbial conglomerate” refers to as a cluster (2 or more) of bacteria made up of two of more different species.

Specific examples show the dechlorination of triclocarban and/or the dechlorination of triclosan comprising contacting a sample containing triclosan the microbial compositions described herein.

### BRIEF DESCRIPTION OF SEVERAL VIEWS OF THE DRAWINGS

FIG. 1: Schematic for Production of Sediment-Free Cultures.

FIG. 2: TCE reductive dechlorination in Sediment-D microcosm.

FIG. 3: TCE reductive dechlorination in Sediment-D microcosm with TCC & TCS.

FIG. 4: TCE reductive dechlorination in Sediment-A microcosm.

FIG. 5: TCE reductive dechlorination in Sediment-A microcosm with TCC & TCS.

FIG. 6: TCE dechlorination in sediment microcosms in the presence or absence of BES. Symbols: ♦, TCE; ▴, trans-DCE; ▪, cis-DCE; x, VC; ∘, ethene.

FIG. 7: TCE dechlorination in sediment-free cultures generated from sediment microcosms. Symbols: ♦, TCE; [symbol] , 1,1-DCE; ▴, trans-DCE; ▪, cis-DCE; x, VC; •, ethene. All data points are averages from triplicate cultures, and error bars represent one standard deviation. When error bars are not visible, they are small and therefore hidden behind the data symbols.

FIG. 8: TCE dechlorination in generation I SCD cultures spiked with TCE and lactate. The initial TCE concentration was 40 μmol per serum bottle. Symbols: ♦, TCE; ▪, cis-DCE; x, VC; •, ethene; ∘, total. All data points are averages from triplicate cultures, and error bars represent one standard deviation. When error bars are not visible, they are small and therefore hidden behind the data symbols.

FIG. 9: TCE dechlorination in generation II SCD cultures. The initial TCE concentration was 26 μmol per serum bottle. Symbols: ♦, TCE; ▪, cis-DCE; x, VC; •, ethene; ∘, total. All data points are average from triplicate cultures, and error bars represent one standard deviation. When error bars are not visible, they are small and therefore hidden behind the data symbols.

FIG. 10: TCE dechlorination in generation III SCD cultures and BDI cultures. Symbols: ♦, TCE; ▪, cis-DCE; x, VC; •, ethene. All data points are average from triplicate cultures, and error bars represent one standard deviation. When error bars are not visible, they are small and therefore hidden behind the data symbols.

FIG. 11: PCE dechlorination in generation II sediment D culture. Symbols: ▴, PCE; ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 12: Microbial community structures of SCD culture, sediment D, SCAT culture and sediment A.

FIG. 13: Proteobacteria in sediment A, sediment D, SCA culture and SCD culture.

FIG. 14: Firmicutes in sediment A, sediment D, SCA culture and SCD culture.

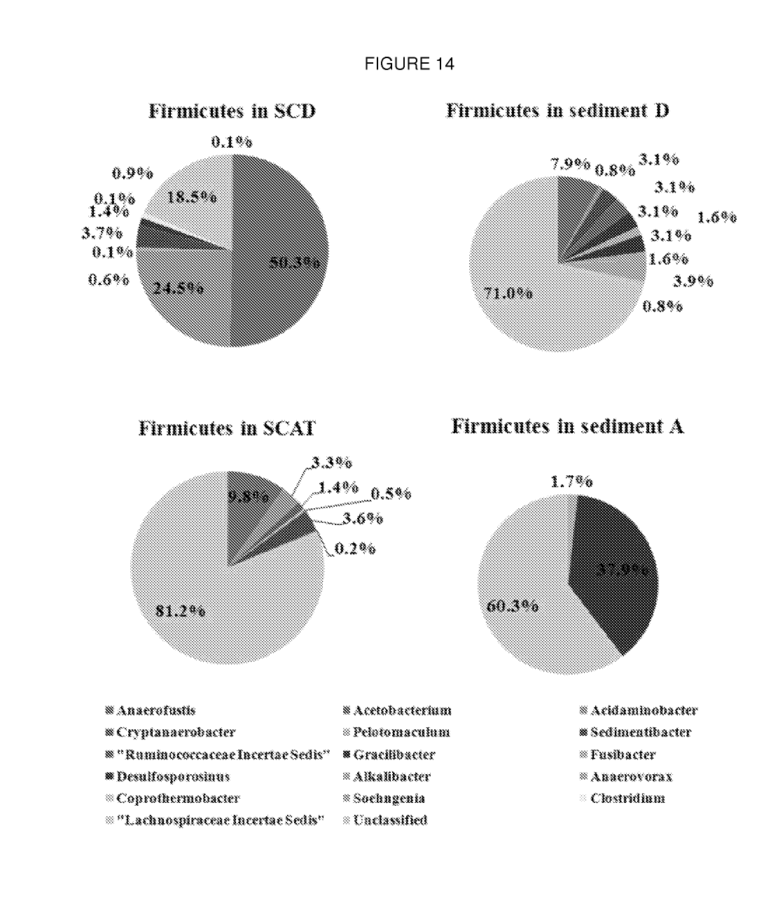

FIG. 15: TCE reductive dechlorination in BDI cultures with 1.5 μM TCC or without TCC. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 16: TCE reductive dechlorination in BDI cultures with 35 μM TCS or without TCS. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 17: TCE reductive dechlorination in SCD cultures with or without 0.15 μM TCC. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 18: TCE reductive dechlorination in SCD cultures with or without 1.5 μM TCC. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 19: TCE reductive dechlorination in SCD cultures with or without 3.5 μM TCS. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 20: TCE reductive dechlorination in SCD cultures with or without 35 μM TCS. Symbols: ♦, TCE; ▪, cis-DCE; x, VC; •, ethene. All data points are averages from triplicate cultures, and error bars represent one standard deviation. When error bars are not visible, they are small and therefore hidden behind the data symbols.

FIG. 21: Growth of general bacteria and Dehalococcoides in the BDI culture without TCC & TCS as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 22: Growth of general bacteria and Dehalococcoides in the BDI culture 1.5 μM TCC as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 23: Growth of general bacteria and Dehalococcoides in the BDI culture 35 μM TCS as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 24: Growth of general bacteria and Dehalococcoides in the SCD culture without TCC & TCS as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 25: Growth of general bacteria and Dehalococcoides in the SCD culture with 1.5 μM TCC as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 26: Growth of general bacteria and Dehalococcoides in the SCD culture with 35 μM TCS as a function of TCE dechlorination reactions. Symbols: ♦, TCE; ▪, cis-DCE; x, VC; •, ethene. All samples were run in triplicate, and standard deviation error bars are included in the figure. Growth trends are estimated using quantitatitve real time PCR targeting general bacterial 16S rRNA genes and Dehalococcoides 16S rRNA genes.

FIG. 27: Cumulative reduction of each chloroethene, represented as mM Cl⁻ produced, and the concentration of Dehalococcoides during successive additions of TCE in an enrichment culture.

FIG. 28: DehaloR̂2 culture grown on lactate as the sole electron donor.

FIG. 29: DehaloR̂2 culture grown on lactate and methanol as electron donors.

FIG. 30: pH and total carbonate concentration without flushing headspace.

FIG. 31: pH and total carbonate concentration after flushing headspace.

### DETAILED DESCRIPTION OF THE INVENTION

There are significant environmental problems associated with the presence of chlorinated compounds in the ground water and soil compositions of areas as a result of dry-cleaning operations, metal degreasing, textile finishing, dyeing and extraction processes in the industry. Remediation of soils and water to remove the chlorinated ethenes and ethanes contaminants has typically involved resin-based ion exchange chromatography, which is an inefficient method because of the bulk of resin required and the fact that the chlorinated compound-loaded resin ultimately must be disposed. Biological methods for remediation or clean-up of the environment are compelling in that they employ natural organisms. Microorganisms have been used to clean-up oil spills, sewage effluence, chlorinated solvents, pesticides and the like. The ability of microorganisms to remove the contaminants from a contaminated site is nature's way of cleaning-up the environment. In the present application, there are provided methods and compositions for using one such set of microorganisms to clean up sites contaminated with chlorinated ethenes.

The present invention is directed to an anaerobic microbial composition comprised at a minimum of a consortium of bioremediative microorganisms where the consortium is made up of a mixture of isolated microbial strains of Chloroflexy, Spirochaetes, Firmicutes, and Proteobacteria. The invention is also directed to methods of using such a microbial composition for the effective dechlorination of at least one of, chlorinated ethenes, chlorinated methanes, or mixtures thereof. In particular, it has been discovered that the use of consortium of isolated microbial strains described herein is particularly effective at the dehalogenation of antimicrobial agents. The microbial consortium of the present invention may be employed for bioremediation to anaerobically biodegrade chlorinated waste, for example, contaminated groundwater or contaminated soil from landfill sites, river beds, lakes, wetlands, and the like.

In certain aspects, the consortium may further comprise one or more additional microbial strains selected from the group consisting of Delta proteobacteria, Beta proteobacteria, Gamaproteobacteria, Acetobacterium, Acidaminobacter, Sedimentibacter, Gracilibacter, Clostridium, Trichlorobacter, Geobacter, Chlostridium, and Dehalococcoides.

In specific embodiments, the microbial composition may comprise a non-naturally occurring consortium of microbial species capable of dechlorination of a compound where the microbial species comprises at least one 16S rDNA nucleic acid sequence that has more than 95% identity to a nucleic acid sequence consisting of SEQ ID NO 1, a nucleic acid sequence consisting of SEQ ID NO 2, or a nucleic acid sequence consisting of SEQ ID NO 3.

In operation, the methods of the present invention involve taking a sample that contains chlorinated ethenes and dechlorinating the sample by inoculating the sample (i.e., contacting, or adding to the sample) with a microbial composition of the present invention under anaerobic conditions to achieve an anaerobic dechlorination of chlorinated ethenes. Accordingly, the methods can be used to dechlorinate any chlorinated ethene The chlorinated ethenes may comprise at least one of cis 1,2-dichloroethene; trans 1,2-dichloroethene; vinyl chloride; or tetrachloroethene. The mixture may also contain chlorinated methane, for example, carbon tetrachloride or chloroform.

The methods of the invention may be used to treat soil that needs dechlorination. Alternatively, chlorinated waste may be dechlorinated by contacting chlorinate ethenes or chlorinated methanes with an isolated bioremediative consortium comprising strains of microorganism comprising Chloroflexy, Spirochaetes, Firmicutes, and Proteobacteria; and anaerobically dechlorinating the at least one of chlorinate ethenes, or chlorinated methanes. The chlorinated waste may comprise contaminated soil or contaminated water.

By way of providing further information regarding the contamination of sites with chlorinated compounds, the applicants provide the following details of the general sources of these contaminants and measures available to remediate contaminated sites/samples.

Chloroethenes, such as tetrachloroethene (PCE), trichloroethene (TCE), dichloroethene (DCE), and vinyl chloride (VC), are among the most common contaminants at National Priority List (NPL) sites (ATSDR, 2007; EPA, 2008a). PCE and TCE are excellent solvents and are widely used in dry-cleaning operations, metal degreasing, textile finishing, dyeing and extraction processes in the industry (Abelson, 1990; McCarty, 1997). Human activities; improper storage, handling, and disposal; and their high chemical stability have resulted in widespread subsurface water and soil contamination across the United States (Abelson, 1990; McCarty, 1997). All chloroethenes are highly toxic, especially VC, a known human carcinogens. VC groundwater contamination mainly results from reductive dechlorination of PCE and TCE under anaerobic conditions (Bradley, 2000; Loffler and Edwards, 2006). Additionally PVC (Polyvinyl chloride) manufacturing operations also release high dissolved concentration of VC into the environment (Hartmans, 1995).

Aside from their production by humans for industrial purposes, PCE and TCE are naturally produced by various marine macroalgae and microalgae (Abrahamsson et al., 1995). Subsurface pyrogenic activity and volcanic eruptions also discharge chloroethenes to marine environments (Gribble, 1994; Gribble, 2003). This is an indication that we cannot neglect natural sources of PCE and TCE. VC was previously believed to be 100% anthropogenic, but Keppler et al. (2002) reported that this highly reactive compound can be formed naturally during the degradation of organic matter in soil (Keppler et al., 2002). Natural production of chloroethenes may stimulate gene expression or enzyme activity for chloroethenes dechlorination and develop beneficial bacteria strains for bioattenuation.

Under anaerobic conditions, PCE and TCE can be biodegraded into less chlorinated or non-chlorinated compounds through reductive dechlorination (See Scheme 1). This dechlorination process can be utilized as a reliable and effective method for bioremediation of contaminated groundwater, soil, and sediment. However, under uncontrolled conditions, reductive dechlorination of PCE and TCE can also be a threat to public health and drinking water safety, as the toxic byproduct VC may accumulate in the subsurface through the abiotic and biotic dechlorination of PCE and TCE (Loffler and Edwards, 2006). In the last 20 years, scientific research on reductive dechlorination has proven that the transformation of chlorinated ethenes into the harmless non-chlorinated end-product, ethene, can be achieved by biological dechlorination reactions in the field. Enhanced in situ bioremediation involves either stimulating the indigenous microorganisms with biodegradating capability by providing sufficient electron donors, acceptors, carbon sources and mineral ions, in a process termed“biostimulation”, or introducing microorganisms with the specific detoxifing capability necessary to degrade the contaminants in the site, “bioaugmentation” (Doong and Wu, 1997). Enhanced in situ bioremediation has been successfully applied for the remediation of chlorinated solvent-contaminated sites (Cupples et al., 2003; Holliger et al., 1998b).

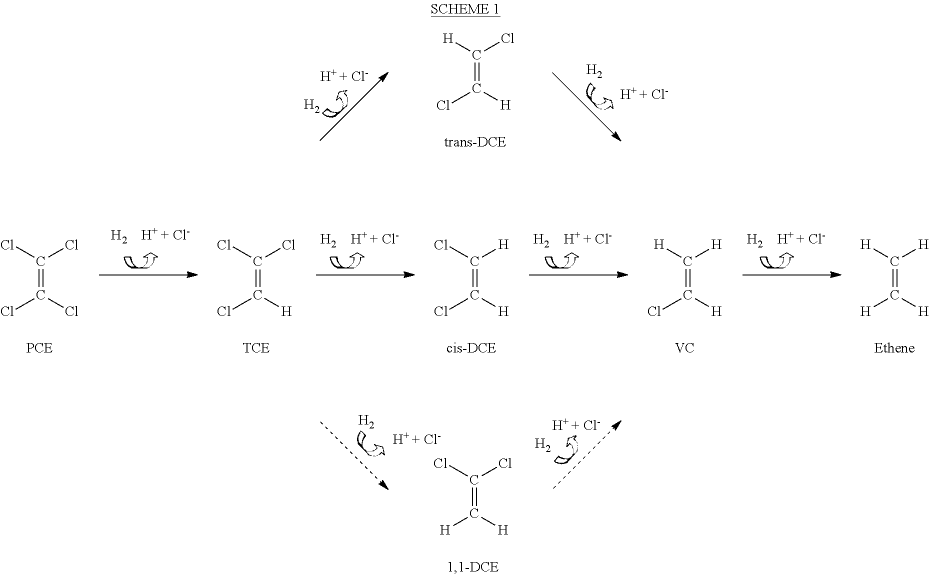

Under anaerobic conditions, diverse groups of bacteria can reductively dechlorinate PCE and TCE into less chlorinated ethenes in a step-wise fashion following the pathway shown in Scheme 1, as a means to capture energy for their growth. Reductive dechlorination beyond cis-DCE to non-chlorinated ethene requires the presence of Dehalococcoides strains (Cupples et al., 2003; He et al., 2005a; He et al., 2003a; He et al., 2003d; Hendrickson et al., 2002; Major et al., 2002; Sung et al., 2006b). Hendrickson et al. (2002) tested samples from 24 chloroethene-dechlorinating sites in North America and Europe for the presence of Dehalococcoides strains by using PCR (polymerase chain reaction) (Hendrickson et al., 2002). They observed a positive correlation between ethene formation and the presence of Dehalococcoides. However, Dehalococcoides species exhibit distinct dechlorinating abilities between each other, and none of the isolated strains can use all kinds of chlorinated ethenes for dehalorespiration (i.e. PCE, TCE, DCEs, VC).

Several microcosms or cultures derived from sediment samples have been reported to have different features than those generated from soil and groundwater. For example, the microcosms derived from river sediments or flat tide sediments produced more trans-DCE than cis-DCE from PCE or TCE reductive dechlorination, and in the microcosms generated from soil and groundwater cis-DCE is usually the main byproduct of TCE reductive dechlorination (Griffin et al., 2004; Kittelmann and Friedrich, 2008c; Miller et al., 2005). trans-DCE is a toxic contaminant (EPA, 2008a) and the complete dechlorination of trans-DCE is necessary for bioremediation. Most reported microcosms which produced more trans-DCE than cis-DCE during reductive dechlorination of PCE or TCE usually stopped at this dechlorination step (Griffin et al., 2004; Kittelmann and Friedrich, 2008b; Miller et al., 2005), suggesting that once the dechlorination on contaminated sites occurs through this pathway natural attenuation and biostimulation may not be effective, therefore bioaugmentation is probably needed. Reductive dechlorination of trans-DCE is now a critical and challenging step requiring the discovery of more effective microbial communities.

Several anaerobic microorganisms can use PCE or TCE as electron acceptors for growth through reductive dechloronation (Field and Sierra-Alvarez, 2004). These microorganisms belong to several genera including Clostridium (Chang et al., 2000), Dehalobacter (Holliger et al., 1998a), Desulfuromonas (Deweerd et al., 1990; Sung et al., 2003), Desulfitobacterium (Bouchard et al., 1996; Sanford et al., 1996), Dehalococcoides (He et al., 2005a; He et al., 2003c; Maymo-Gatell et al., 1997; Sung et al., 2006b), Enterobacter (Sharma and McCarty, 1996), and Sulfurospirillum (formerly Dehalospirillum) (Luijten et al., 2003; Scholzmuramatsu et al., 1995). Their electron-donor requirements, kinetics, end points of dechlorination, and maximum tolerable concentrations of chlorinated ethenes are different (Aulenta et al., 2006). Several dechlorinators are quite restrictive because of their electron-donor requirements, such as Dehalobacter and Dehalococcoides, which can only utilize H₂ as electron donor (He et al., 2005a; He et al., 2003c; Holliger et al., 1998a; Maymo-Gatell et al., 1997; Sung et al., 2006b). On the other hand, other species (such as Sulfurospirillum, Desulfitobacterium) can use a broad spectrum of electron donors (Bouchard et al., 1996; Luijten et al., 2003; Sanford et al., 1996; Scholzmuramatsu et al., 1995). Desulforomonas spp. is special in that they are capable of utilizing acetate as an electron donor for reductive dechlorination (Deweerd et al., 1990; Sung et al., 2003). However, the Dehalococcoides is the most unique genus because it can drive the dechlorination of chloroethenes to harmless ethene (He et al., 2005a; He et al., 2003c; Maymo-Gatell et al., 1997; Sung et al., 2006b). For this reason, many research groups have undertaken to isolate and study the functional roles of Dehalococcoides strains in reductive dechlorination processes.

In 1997, Maymo-Gatell et al. reported the isolation and characterization of the first strain of Dehalococcoides and named it Dehalococcoides ethenogenes strain 195 (Maymo-Gatell et al., 1997). This strain metabolically dechlorinates PCE to VC, but VC transformation to ethene is a slow cometabolic process. Because VC is considered the most toxic chloroethene, being a known human carcinogen, finding a Dehalococcoides strain that could dechlorinate VC to ethene became extremely important. Without this accomplishment, VC will easily accumulate in contaminated sites by natural biotic and abiotic processes. In 2003, He et al. successfully isolated a Dehalococcoides strain which could use VC as a growth-supporting electron acceptor (He et al., 2003c), after which, different strains of Dehalococcoides, such as VS (Müller et al., 2004), FL2 (He et al., 2005a), and GT (Sung et al., 2006b), were isolated in succession. Strain FL2 can respire TCE and DCEs. Strain BAV1 and VS can only use DCEs and VC as electron acceptors for growth, and finally, strain GT can use TCE, DCEs and VC as growth-supporting electron acceptors, but it can hardly use PCE. Moreover, He et al. and Kube et al. showed that Dehalococcoides strains share a similar 16S rRNA gene sequence (He et al., 2005a; Kube et al., 2005). Therefore, 16S rRNA gene-based analyses are insufficient to predict dechlorination activity and distinguish between members of the Dehalococcoides group. Analysis of the genes that encode reductive dehalogenase enzymes (RDases) can overcome this limitation, and researchers now use them as a fingerprint to indicate the presence of different Dehalococcoides strains (Holmes et al., 2006; Ritalahti et al., 2006; Sung et al., 2006b). Up to now, the characterized RDase encoding genes in Dehalococcoides strains include tceA, bvcA, and vcrA. Strains FL2 and 195 contain the tceA gene (Magnuson et al., 2000), and one characteristic RDase reported for Strain BAV1 which is absent in strains FL2, 195, VS and GT is bvcA. The vcrA gene first described in strain VS (Müller et al., 2004) is also present in strain GT (Sung et al., 2006b).

According to current understanding, Dehalococcoides strains are strictly hydrogenotrophic chlororespirers, and thus, maintaining Dehalococcoides in pure culture remains a challenge (He et al., 2003d; Maymo-Gatell et al., 1997). Dehalococcoides exhibit greater growth potential in mixed cultures, so mixed cultures are a better choice for the bioremediation of chlorinated ethenes (Duhamel et al., 2004; Loffler and Edwards, 2006).

TABLE 1
Dehalococcoides strains and reductive dehalogenase genes^{a}.

| | Electron | | | | |
|---|---|---|---|---|---|
| Strain | acceptor | bvcA | tceA | vcrA | References |
| Etheno- | PCE, TCE, | − | + | − | (Krajmalnik-Brown et al., |
| genes | DCE | | | | 2004; Müller et al., 2004; |
| 195 | | | | | Magnuson et al., 2000) |
| FL2 | TCE, DCE | − | + | − | (He et al., 2005a; |
| | | | | | Krajmalnik-Brown et al., |
| | | | | | 2004; Müller et al., 2004; |
| | | | | | Magnuson et al., 2000) |
| BAV1 | DCE, VC | + | − | − | (He et al., 2003a; |
| | | | | | Krajmalnik-Brown et al., |
| | | | | | 2004) |
| GT | TCE, DCE, | − | − | + | (Sung et al., 2006b) |
| | VC | | | | |
| VS | DCE, VC | − | − | + | (Krajmalnik-Brown et al., |
| | | | | | 2004; Müller et al., 2004) |
| CBDB1 | Chlorinated | − | − | − | (Holscher et al., 2004) |
| | benzenes | | | | |

^{a}+ indicates the reductive dehalogenase gene has been reported present; − indicates the reductive dehalogenase gene has been reported absent.

KB-1 and Bio-Dechlor INOCULUM (BDI) are two commercially available and widely used reductive-dechlorinating mixed cultures.

KB-1 was enriched from soil and groundwater obtained from a Southern Ontario TCE contaminated site in 1996 (Major et al., 2002). Through continuous transfers (10% v/v) into sterile anaerobic medium, KB-1 culture was able to reductively dechlorinate high concentrations of TCE and PCE. This culture was routinely fed 300 μM TCE and 1.5 mM methanol every two weeks. The maximum cell density of the culture was about 10⁸ cells per mL, and the corresponding maximum protein concentration was about 40 mg/L (Major et al., 2002). Because KB-1 has performed so well, and no pathogenic bacteria are present in the culture, Major et al. used it for a pilot-scale field test to evaluate bioremediation performance of tetrachloroethene (PCE) to ethene at Kelly Air Force Base. Within 200 days, the concentrations of PCE, TCE, and cis-DCE in the field decreased from 6 μM to lower than 0.05 μM (Major et al., 2002). After that, KB-1 was successfully used for field biostimulation and bioaugmentation at the Rugårdsvej site 234 in Odense, Denmark (Scheutz et al., 2008), the Launch Complex 34 at Cape Canaveral Air Force Station (Hood et al., 2008) and the Launch Complex 34 at Cape Canaveral, Fla. (SiREM, 2009).

BDI was developed by Dr. Frank Loffler's research group and has been successfully used for bioaugmentation at chlorinated ethene-contaminated sites (Ritalahti et al., 2005) and it was also used in inhibition tests for tween 80 (polyoxyethylene [20] sorbitan monooleate) (Amos et al., 2007) and oxygen (Amos et al., 2008). Sung (2005b) developed BDI from two pure Dehalococcoides species (strain BAV1 and FL2) and three PCE reducing enrichment cultures, H7-PCE, H5-PCE, and FMC-PCE. He provided lactate or H₂ as electron donor, and no methane was produced in this culture (Sung, 2005b).

Several other cultures currently are being used for bioremediation, such as the SDC-9 developed by Shaw Environmental, Inc (Schaefer et al., 2009) and the Pinellas culture developed by General Electric Company (Ellis et al., 2000).

Factors that Influence Reductive Dechlorination

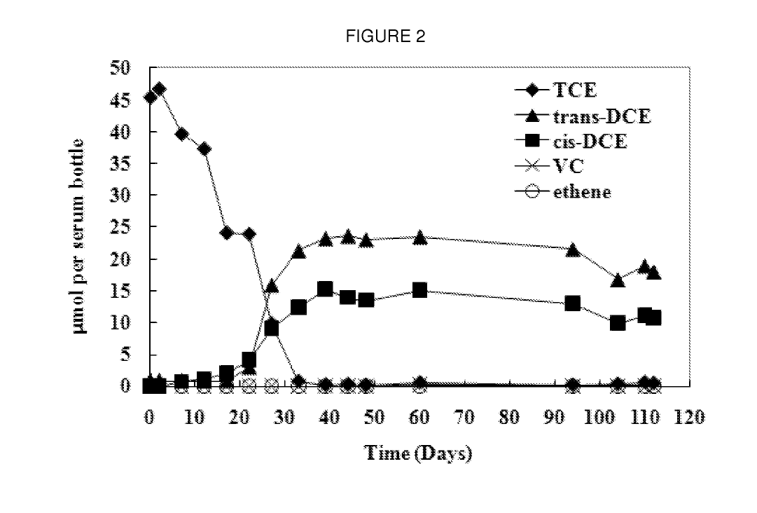

Hydrogen Concentration. Dehalococcoides can only utilize H₂ as its exogenous electron donor (recall FIG. 2.1); hence, hydrogen plays an important role in the process of reductive dechlorination (Holliger et al., 1998b; Loffler and Edwards, 2006). Dechlorination rates have shown a Monod or Michaelis-Menten dependence on H₂ concentration (Ballapragada et al., 1997; Cupples et al., 2004). Ballapragada et al. (1997) calculated the H₂ concentration given one half the maximum rates (Kₛ) of H₂ utilization for each step of PCE reductive dechlorination in a fluidized bed reactor. The Kₛ values ranged from 9 to 21 nmol/L (Ballapragada et al., 1997). Furthermore, Cupples et al. (2004) found that Kₛ values for H₂ utilization for cis-DCE and VC dechlorination by Dehalococcoides strain VS were equal to 7±2 nmol/L (Cupples et al., 2004). Recently, Chung et al. (2008) utilized a H₂-based membrane-biofilm reactor (MBfR) to remove TCE. 93% of TCE was reductively dechlorinated to ethene when the H₂ pressure was 2.5 psi (0.17 atm) in the lumen of the hollow fiber delivering H₂ to the reactor. The H₂ concentration in the MBfR system usually was as low as 10 μg/L (Chung et al., 2008), so addition of H₂ is critical, but a higher concentration may not so important.

Electron Donor and pH. In the process of reductive dechlorination, chlorinated ethenes lose chlorines in a stepwise manner (as shown in Scheme 1), and every step of reductive dechlorination requires hydrogen and produces hydrochloric acid. This may cause two problems: (1) hydrogen delivery; (2) low pH. A low hydrogen delivery rate may inhibit the performance of Dehalococcoides and can waste large amounts of hydrogen (hydrogen has low solubility in water, about 1 mM at 1 atm). Furthermore, in the process of PCE reductive dechlorination, four moles of hydrochloric acid are produced per mole of PCE, which will significantly depress the pH of the system. Obligate anaerobic microorganisms usually grow well between pH 6.8 and pH 7.2, which means pH is an important limiting factor for anaerobic microorganisms.

According to water chemistry and reaction stoichiometry, maintaining a pH of 6.5 or above in the water would permit dechlorination of no more than 3.3 mM chloride (1.1 mM TCE) with an initial bicarbonate alkalinity of 400 mg/L (as CaCO₃) (McCarty et al., 2007). However, in pure water or tap water the bicarbonate alkalinity is not likely to be 400 mg/L (as CaCO₃) or more alkaline. Therefore, adding buffer solution is necessary for the bioremediation of chlorinated ethenes, especially for removal of chloroethenes dense non-aqueous-phase liquid (DNAPL) (McCarty et al., 2007).

Chung et al. (2008) utilized an MBfR to remove TCE and excellently solved the problem of hydrogen delivery with almost 100% hydrogen utilization rate and a 2.5 psi (0.17 atm) standard H₂ pressure in the reactor, no information regarding pH was mentioned, although the TCE concentration in the influent was 7.6 uM which was much lower than 1.1 mM (Chung et al., 2008).

Temperature. Temperature is an important factor to all living organisms, and a certain or ideal temperature can improve the performance of biodegradation microorganisms. Temperature is a critical factor in chlorinated solvent bioremediation for two reasons: 1) choosing a suitable temperature for Dehalococcoides or other dechlorinating microorganisms will increase the dechlorinating rate of reductive dechlorination process in bench-scale bioreactors or in microcosms; 2) chlorinated solvents can exist as DNAPLs in groundwater which are difficult to remediate due to mass transfer limitation, and thermal treatment can efficiently improve the mass transfer and removal of most chlorinated contaminants (Heron et al., 2005). The temperature will remain high for months to years after thermal treatment, and further bioremediation may still be necessary for removing the residual contamination. Friis et al. utilized microcosms to evaluate bioaugmentation after a field scale thermal treatment of a TCE contaminated aquifer and found that 30° C. was the optimal temperature for complete dechlorination using the KB-1 culture (Friis et al., 2007a; Friis et al., 2007b). However, the thermal treatment is an expensive remediation method, and the groundwater temperature is typically around 15° C. and much lower than room temperature. Reductive dechlorinating cultures are usually maintained at room temperature or 30° C. in the laboratory, and the Dehalococcoides dominated microbial communities will be inactivated at low temperature (Friis et al., 2007a), therefore, the subsurface bioremediation of TCE contamination may be affected by low temperature in the fields.

There may be other factors which will affect the reductive dechlorination process since the natural conditions of groundwater, soil and sediment are various and complex. For examples, some biocides, such as triclocarban (TCC) and triclosan (TCS) may exist in sediments, however, up to now the effect of TCC and TCS on reductive dechlorination of chloroethenes has not been studies yet.

### Triclocarban and Triclosan

Properties and Toxicity of Triclocarban and Triclosan. Triclocarban, 3-(4-chlorophenyl)-1-(3,4-dichlorphenyl) urea or 3,4,4′-trichlorocarbanilide (also called TCC, cutisan, solubacter, trichlorocarbanilide, etc.), is a polychlorinated phenyl urea (Scheme 2) that has antibacterial and antifungal properties (Consortium, 2002). It has been globally used as an antimicrobial and preservative in bar and liquid soaps, body washes, and other personal care products at the levels of up to 5 wt % since the 1960s (Yackovich et al., 1986). The most widespread use of TCC is in antimicrobial bar soaps (Consortium, 2002). Triclosan (TCS, 5-chloro-2-[2,4-dichloro-phenoxy]-phenol) is also a broad spectrum antimicrobial and preservative agent that is widely used in personal-care products such as soaps, toothpastes, and cosmetics (EPA, 2008b). The total amount of TCC and TCS that are released into the U.S. environment exceed 600,000 kg/yr, and this number may be as high as 10,000,000 kg/yr according to the supporting information Table 51 (EPA, 2003). The physical and chemical properties of TCC and TCS are listed in Table 2.3.

TCC and TCS are slightly soluble in water (0.65 mg/L and 4.6 mg/L respectively at 25° C.) and are non-volatile (Ying et al., 2007). They have a high partition coefficient, which implies that it is highly possible for TCC and TCS to partition into soil or sediment. They are fat-soluble and easily cross cell membranes. When triclosan enters into cell, it blocks the active site of the enzyme enoyl-acyl carrier-protein reductase (ENR), which is absent in humans and other organisms (Levy et al., 1999; McMurray et al., 1998). This enzyme is responsible of producing fatty acids for the construction of cell membranes (Levy et al., 1999; McMurray et al., 1998). Because triclocarban's structure is similar to triclosan, some researchers think they may share similar modes of action (Ying et al., 2007).

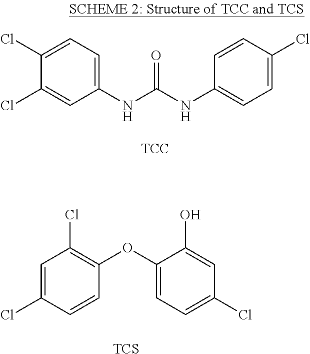

A study of TCC revealed that, because of its antibacterial properties, it could reduce the number of Methicillin-resistant Staphylococcus aureus (MRSA) in patients with moderately severe atopic dermatitis (Breneman et al., 2000) and vancomycin-resistant enterococcus (VRE) (Sutler and Russell, 1999). TCC is not effective at inhibiting Gram-negative microorganisms growth, but can effectively inhibit Gram-positive bacteria, such as staphylococcus, even at the relatively low concentration of 100-200 μg/L (Gledhill, 1975). However, 1000 mg/L is needed to inhibit Gram-negative species and fungi (Gledhill, 1975). In Minimum Inhibitory Concentrations (MIC) experiments TCC only showed activity against MRSA, but not E. coli and P. aeruginosa (Walsh et al., 2003).

TABLE 2
Physical and chemical properties of TCC and TCS
(Consortium, 2002; Halden and Paull, 2005; Ying et al., 2007).

| Property | Triclocarban | Triclosan | Unit |
|---|---|---|---|
| CAS number | 101-20-2 | 3380-34-5 | |
| Molecular formula | C₁₃H₉Cl₃N₂O | C₁₂H₇Cl₃O₂ | |
| Molecular weight | 315.6 | 289.54 | |
| Melting point | 250 | 180 | ° C. |
| Boiling point | 434.57 | 120 | ° C. |
| Vapor pressure | 3.61 × 10⁻⁹ | 4.65 × 10⁻⁶ | mm Hg at 25° C. |
| Water solubility^{a} | 0.65-1.55 | 1.97-4.62 | mg/L at 25° C. |
| | 0.05 | <1 | mg/L at 20° C. |
| Partition Coefficient | 4.9 | 4.8 | Log P_{ow} (at 25° C., |
| | | | pH 7) |

^{a}The water solubility was calculated using PBT Profiler and ECOSAR.

Different kinds of indicator organisms (such as algae, aquatic invertebrates and fish) were used in acute and chronic ecotoxicity studies of TCC. The Predicted No Effect Concentration (PNEC) and the Predicted Environmental Concentrations (PEC) determined for TCC are 0.146 μg/L and 0.0013-0.050 μg/L (depending on the assessment scenario), respectively (Consortium, 2002). Therefore, the EPA website reports that the ratio of PEC/PNEC is lower than 1 (0.009 to 0.34), making the potential for adverse environmental effects of TCC low. Additionally, TCC is of low acute and chronic toxicity and has an acceptable human safety profile for the applications of personal cleansing products.

However, Chen et al. reported that TCC can act as an endocrine disruptor (Chen et al., 2008). TCC can initiate an acute increase of gene expression in human cells which is normally regulated by testosterone. Testosterone-dependent organs, such as the prostate gland, grew abnormally large in male rats fed with TCC (Chen et al., 2008). Additionally, TCC can be toxic to aquatic organisms at a concentration of 90 mg/L according to PBT Profiler (Ying et al., 2007). Nolen and Dierckman reported TCC toxicity for diverse mammals. At increased TCC levels, reproduction and offspring survival rates of rats and rabbits decreased (Nolen and Dierckman, 1979). Furthermore, TCC is known to cause methemoglobinemia (“Blue Baby” Syndrome) in humans (Johnson et al., 1963). During the degradation of TCC, the carbon-nitrogen bonds are cleaved, producing the intermediate byproduct 3,4-dichloroaniline. The presence of this byproduct may increase the possibility of methemolgobinemia (Johnson et al., 1963; Ponte et al., 1974) and induce ecotoxicity, genotoxicity, and hematotoxicity (Boehncke et al., 2003; Gledhill, 1975).

Algae are sensitive to TCS also. The 96-h effective concentration (EC₅₀) for the growth of algal species Scenedesmus subspicatus is 1.4 μg/L, and the 96-h no-observed effect concentration (NOEC) is 0.69 μg/L (Orvos et al., 2002). Animals are less sensitive to TCS. A median EC₅₀ of 350 μg/L and a NOEC of 34 μg/L have been reported for Rainbow trout (Adolfsson-Erici et al., 2002; Orvos et al., 2002). Veldhoen et al. showed that low concentrations of TCS 0.15±0.03 μg/L can act as an endocrine disruptor in the North American bullfrog and can alter the rate of thyroid hormone-mediated postembryonic anuran development (Veldhoen et al., 2006). The proposed toxicity mechanism is that triclosan may block the metabolism of thyroid hormone so normal hormones cannot be utilized (Veldhoen et al., 2006).

Fate and Transport of Triclocarban and Triclosan in Environment. Because TCC and TCS are slightly soluble in the water, non-volatile and have high partition coefficients, they are likely to be adsorbed to wastewater sludge (biosolids or sediments) (Halden and Paull, 2005; Heidler et al., 2006). If this sludge is disposed on the land, it will provide a route of transportation for TCC and TCS into the environment. Additionally, due to the incomplete degradation of TCC and TCS in the wastewater treatment plant, trace levels of TCC and TCS still remain in the effluent, which ultimately make it into rivers and lakes (Halden and Paull, 2005; Kolpin et al., 2002). Thus, TCC and TCS could potentially make their way to the groundwater. Furthermore, researchers have proved that TCC and TCS exists in the influent, effluent and sludge of wastewater treatment plants. The TCC or TCS concentration are in the mg/kg dry weight level in wastewater sludge (Halden and Paull, 2004; Halden and Paull, 2005; Heidler et al., 2006; Ying et al., 2007).

Ying et al. used environmental fate models, STPWIN32 (modeling software for removal in wastewater treatment), Level III fugacity model and PBT Profiler, to predict the fate of TCC and TCS in the environment (Ying et al., 2007). All models showed that more than 80% of TCC mass partitioned into sludge and sediment, and 7%-11.8% TCC stayed in water and 0.65% could be biodegraded; for TCS, 7%-13.9% TCS stayed in water, and 0.61% could be biodegraded, and as with TCC, most TCS partitioned into sludge and sediment (Ying et al., 2007).

Bioaccumulation and Biodegradation of Triclocarban and Triclosan. Although a proportion of TCC and TCS can be hydrolyzed or biodegraded to CO₂, nitrate, and chloride, the trace amounts of TCC and TCS are very difficult to remove and thus can bioaccumulate in algae or other organisms.

Ying et al. utilized the PBT Profiler to estimated the fish bioconcentration factor values (log BCF 3.074) which suggested that bioaccumulation of TCC and TCS is possible (Ying et al., 2007). Coogan et al. studied algal bioaccumulation of TCC, TCS and methyl-triclosan in a North Texas wastewater treatment plant receiving stream and proved that there are high bioaccumulated concentrations of TCC and TCS (Coogan et al., 2007). TCS has been found in both the bile of fish living downstream of wastewater treatment plants and in human breast milk (Adolfsson-Erici et al., 2002). TCC and TCS persisted in water samples with concentrations of 80-190 ng/L and 60-120 ng/L, and higher concentrations were seen in algae of 219-401 μg/L and 109-146 μg/L for TCC and TCS, respectively (Coogan et al., 2007). This indicates that TCC is likely bioaccumulating in algae. However, the effects of the bioaccumulation of TCC and TCS in algae are still not clear.

Early in the 1975, Gledhill tested the biodegradation of TCC in the sewage and activated sludge system utilizing shake flask experiments and a bench scale continuous flow activated sludge (CFAS) system, and be obtained up to 96% removal of TCC (Gledhill, 1975). Therefore, he thought that a relatively small amount of TCC would accumulate in the environment. According to those experiments, EPA believed that TCC could be “inherently biodegradable and extensively removed (98%) during wastewater treatment through a combination of sorption and biodegradation process” (Consortium, 2002). However, when the concentration of TCC was 20 μg/L or 2000 μg/L, Gledhill observed only 60% or 70% biodegradation (Gledhill, 1975). This indicated that biodegradation was not effectively achieved at extremely low or high concentration. In fact, according to the environment modeling and field measurement, Predicted Environmental Concentrations (PEC) of TCC are 0.0013 μg/L to 0.05 μg/L (Consortium, 2002). This is much lower than the previous 20 μg/L reported. Furthermore, most real wastewater systems are CFAS systems, and when Gledhill used the bench scale CFAS system he could just get 25.9% biodegradation of the dichloroaniline ring and 56% biodegradation of the p-chloroaniline ring. Almost 35% of the TCC was adsorbed by activated sludge instead of being biodegraded (Gledhill, 1975). Although Gledhill's research demonstrated that TCC was biodegradable, his experiment could not strongly prove that real wastewater treatment plants can effectively biodegrade all TCC.

In 1988, Audu and Heyn studied the hydrolysis rates and half-lives of TCC (Audu and Heyn, 1988). These were determined to be 3.8±0.3×10⁻⁴ day⁻¹ and 5.0 year, respectively. The hydrolysis of TCC was extremely slow regardless of the solution being alkaline, neutral, or acidic. This proved that TCC could not be biodegraded quickly. Furthermore, Ying et al. (2007) used the environmental fate models developed by EPA to assess the biodegradability of TCC and TCS. All of the six different BIOWIN models determined that TCC and TCS do not biodegrade fast, and have primary biodegradation half-lives of weeks and ultimate biodegradation half-lives of months (Ying et al., 2007).

So far, most of the research on TCC biodegradation has been focused on activated sludge wastewater treatment which promotes both aerobic and anaerobic biodegradation and adsorption (Gledhill, 1975; Heidler et al., 2006; Ying et al., 2007). The primary biodegradation byproducts of TCC were chloroaniline components, but the detailed biodegradation processes or pathways, such as which bacteria or enzymes are responsible for the biodegradation of TCC, have not been reported in the literature.

Biodegradation of TCS has been studied extensively in activated sludge processes. Federle et al. found that more than 80% of TCS was removed by a continuous activated sludge laboratory study. They attributed this removal to a biodegradation process in activated sludge treatment (Federle et al., 2002). Variable removal rates of TCS in activated sludge treatment have been reported (Bester, 2003; Stasinakis et al., 2007; Thompson et al., 2005; Yu et al., 2006). In a German wastewater treatment plant, approximately 30% of TCS was adsorbed to the sludge, and the concentrations of TCS ranged from 0.4 to 8.8 mg/kg in sludge samples from 20 sewage treatment plants in Germany (Bester, 2003). In continuous-flow activated sludge systems, 90% TCS was removed by biodegradation and sorption with TCS concentrations ranging from 0.5 to 2 mg/L, this research also proved that heterotrophic microorganisms are less sensitive to 2 mg/L TCS than nitrifiers (Stasinakis et al., 2007).

In aerobic sludge digestion processes, degradation of TCS was observed, but little removal of TCS was detected in anaerobic digestion (McAvoy et al., 2002). Ying et al. studied the aerobic and anaerobic biodegradation of TCC and TCS in soil (Ying et al., 2007). Under aerobic conditions, slow biodegradation of TCC and TCS was detected, and the half-life for TCC and TCS were estimated to be 108 days and 18 days, respectively, based on first order kinetic reaction. Under anaerobic conditions, little biodegradation of TCC and TCS was detected within 70 days. The reasons for the persistence of TCC and TCS in anaerobic soil was not clear (Ying et al., 2007). According to these studies, it seems that aerobic biodegradation of TCS is possible and fast, and anaerobic biodegradation of TCS is extremely slow in the soil or sediment. However, Miller et al. (2008) examined the environmental fate of TCC and TCS in the estuarine sediments from Chesapeake Bay in Maryland, and found significant quantities of the byproducts of TCC reductive dechlorination, such as dichlorocarbanilide (DCC), monochlorocarbanilide (MCC) and nonchlorinated carbanilide (NCC), in the aged deep sediment (Miller et al., 2008). Because the TCC:DCC ratio in the surface water was 70:1 which was much higher than that in the sediment, 1:5, anaerobic dechlorination of TCC may be ongoing in situ, and dehalorespiring microorganisms may be involved in this process (Miller et al., 2008). If that is true, these dehalorespiring microorganisms will possibly dechlorinate other chlorinated compounds, such as TCE. Thus, future research is needed to investigate the anaerobic dechlorination of TCC and TCS and the effect of TCC and TCS on other anaerobic biodegradation processes.

### Example 1

### TCE Reductive Dechlorination in Sediment Microcosms and in Sediment-Free Dechlorinating Cultures

In this study, four microcosms were set up with sediments obtained from Chesapeake Bay (CB) located near Baltimore, Md., where reductive dechlorination byproducts of TCC were detected (Miller et al., 2008). Anaerobic microorganisms were selectively enriched by establishing microcosms in serum bottles. Sediment-free cultures from the sediment microcosms were developed. The potential for reductive dechlorination of chlorinated ethenes by these sediment microcosms and sediment-free cultures was then explored. Based on the development of sediment-free cultures enriched cultures capable of reductively dechlorinating TCE at faster rates than the ones reported in the literature for the available dechlorinating cultures were thus isolated. Furthermore, the microbial community structures in sediment and sediment-free cultures by were investigated pyrosequencing targeting a conserved region in the 16S rRNA gene.

Materials and Methods

Chemicals. PCE and TCE were purchased from Sigma-Aldrich Co. (St. Louis, Mo.). cis-DCE, trans-DCE and 1,1-DCE were purchased from Supelco Co. (Bellefonte, Pa.). Gaseous VC was obtained from Fluka Chemical Corp. (Ronkinkoma, N.Y.), and ethenes were purchased from Scott Specialty Gases (Durham, N.C.). TCC was obtained from Sigma-Aldrich Co. (St. Louis, Mo.) with 99% purity and TCS was purchased from TCI America (Portland, Oreg.) with 96% purity. BES (2-bromoethanesulfonic acid) was obtained from Sigma-Aldrich Co. (St. Louis, Mo.). Vitamin B₁₂ was purchased from Sigma-Aldrich Co. (St. Louis, Mo.) and the mixed vitamin solution was obtained from ATCC (Catalog No. MD-VS, Manassas, Va.).

Microcosms Setup. The sediment was obtained from Back River, a residential tributary of Chesapeake Bay (CB) located near Baltimore, Md. This river receives effluent from a wastewater treatment plant, and reductive dechlorination byproducts of TCC (i.e. DCC, MCC and NCC) were found in the sediment samples (Miller et al., 2008). This location and the plant have been described in detail previously (Heidler and Halden, 2007; Miller et al., 2008). For the present study, two sediment cores, A and D, were taken from CB, and were kept in airtight plastic tubes at 4° C.

Upon arrival of the sediment cores to the laboratory, the sediment samples from different depths of core D were mixed homogeneously in the anaerobic glove chamber and stored in sterile Mason jars at 4° C. For core A, the same procedure was followed.

In this study, microcosm is defined as: anaerobic airtight serum bottles with sediment samples, anaerobic medium, electron donor, and a chlorinated compound used as an electron acceptor. Four microcosms were set up in the anaerobic glove chamber with H₂ concentration of 3% (Coy laboratory products Inc. Grass Lake, Mich.). Ten gram of homogeneously mixed sediment from either core A or core D were transferred independently into a 160 ml sterile serum bottle, and 90 ml anaerobic media with vitamin solution and 2 mM lactate were added. One hundred μL of 20 mg/L TCC stock solution in methanol and 100 μL of 20 mg/L TCS stock solution were added by pipette (Eppendorf, Calif.) to two of the four microcosms. Microcosms were capped with thick black butyl rubber stoppers and aluminum crimps. 4.5 μL pure TCE was injected with airtight syringe (Hamilton Company, Reno, Nev.) into each serum bottle. On day 130, in order to inhibit methane production in the sediment microcosms, 25 mM BES was added in to sediment-A microcosm and sediment-D microcosm with TCC and TCS with airtight syringe (Hamilton Company, Reno, Nev.). Microcosms were stored in the dark and incubated at 30° C.

In order to explore if there was any abiotic dechlorination of TCE in the sediment microcosms, another two microcosms were set up with autoclaved sediment as abiotic controls for TCE dechlorination.

Anaerobic media were prepared using the Hungate technique and described by Löffler et al. (Löffler et al., 2005).

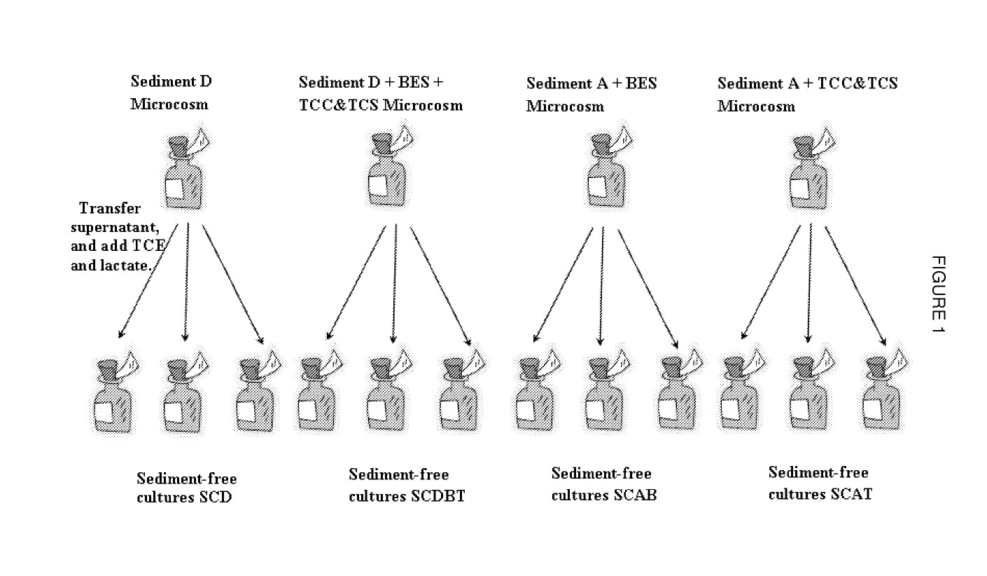

Production of Sediment-Free Cultures. To establish sediment-free cultures from core A and core D sediment microcosms, the microcosms were shaken vigorously, and the solids were allowed to settle down. Then, using 1-inch, 21-gauge sterile needles, 1% (v/v) liquid (10 ml) were transferred into 89 ml sterile medium with the following contents: anaerobic medium, ATCC vitamin mix, and vitamin B12, in 160-ml serum bottles. Then, 1 ml each of 500-mM lactate stock solution and 35-μL TCE stock solution (1 mol/L in methanol) were added to form a 100 ml culture in 160-ml serum bottle. Three sediment cultures were derived from each sediment microcosm, and 12 sediment-free cultures were produced (FIG. 1). For organizational purposes, we refer to the sediment-free cultures generated from sediment-A microcosms as SCA and the sediment free cultures generated from sediment-D microcosms as DehaloR̂2, in the figures herein, the DehaloR̂2 are referred to as “SCD”. Thus, the term “SCD” and DehaloR̂2 are used interchangeably herein as the composition “SCD” was renamed “DehaloR̂2” during the course of investigation. The sediment-free cultures were kept in the dark and incubated at 30° C. After all the chlorinated electron acceptors were consumed, the cultures were re-spiked with TCE and electron donors.

Analytical Methods. The concentrations of PCE, TCE, and their reductive-dechlorinating byproducts, 1,1-DCE, cis-DCE, trans-DCE, VC, and ethene, were quantified by gas chromatography with a flame ionization detector (GC-FID).

Two hundred μL headspace samples were withdrawn from 160 ml serum bottle with 500 μL gas-tight syringes (Hamilton Company, Reno, Nev.) and analyzed by a Shimadzu GC-2010 (Columbia, Md.) with an Rt™-QSPLOT capillary column (30 m×0.32 mm×10 μm, Restek, Bellefonte, Pa.) and a flame ionization detector (FID). The initial oven temperature was 110° C. and was held for 5 min, and then raised with a gradient of 10° C./min to 150° C., a second ramp at 20° C./min to 200° C., and a third ramp of 5° C./min to 220° C., after that, the oven temperature was held at 220° C. for 5 min. The temperature of the FID and injector were 240° C. Ultra high purity helium was the carrier gas, and ultra high purity hydrogen and zero grade air were used for the FID.

Calibration curves for chlorinated compounds were determined based on known masses of PCE, TCE, 1,1-DCE, cis-DCE and trans-DCE added to 160-mL serum bottles containing 100 mL of distilled-deionized water and equilibrated at 25° C. overnight. Different volumes of 100 ppm and 1000 ppm VC and ethene gas were injected directly into GC by airtight syringe to make the calibration curves. The aqueous concentrations of PCE, TCE and their reaction products were calculated by using reported Henry's constants on EPA website (Washington and Weaver, 2006).

DNA Extraction. DNA isolation was conducted using the FASTID kit. Mio Bio Bead Tubes and 10% SDS solution were used to increase lysis and improve the DNA yields.

For the sediment, 0.4-g samples were weighed, then placed in microcentrifuge tubes, and used for DNA isolation. For SCA and DehaloR̂2 cultures, the serum bottles were shaken vigorously first, and then 10 ml of the mixed liquid was removed with a sterile syringe and dispensed into 10 1.5 ml micro centrifuge tubes. These were then centrifuged at 13,000 rpm for 15 min in order to make a pellet. The pellet was then used for DNA extraction. DNA samples were stored at −20° C.

3.1.6 PCR and Quantitative Real-Time PCR. PCR was performed for analysis of general bacteria and Dehalococcoides in sediment microcosms and sediment cultures. The final volume of PCR reactions was 20 μL, and the final concentrations of each chemical in a single reaction tube was: Mg(Acetate)₂ 2.5 mM (Eppendorf, Calif.), Master Mix (1×) (Eppendorf, Calif.), Primers (0.5 μM each), and 2 μL 10 ng/μL of DNA samples. PCR water was used as a negative control, and Dehalococcoides strain BAV1 16S rRNA genes were used as positive control.

PCR conditions for general bacteria included an initial denaturation step at 92° C. for 2 min, followed by 30 cycles at 94° C. for 30 s alternated with the annealing temperature of 55° C. for 45 s, and 72° C. for 2 min. The amplification products were separated by horizontal gel electrophoresis on a 1% agarose gel (Amresco, Solon, Ohio) stained with ethidium bromide (Sigma Chemical Co., St. Louis, Mo.) and visualized under UV light. The gel images were captured using a gel documentation system (GelDOC2000, Biorad, Calif.). The general bacterial primers used for 16S rRNA gene were 8F (5′-AGAGTTTGATCCTGGCTCAG-3′) and 1525R (5′-AAGGAGGTGATCCAGCCGCA-3′).

PCR conditions for a Dehalococcoides 16S rDNA amplification included an initial denaturation at 94° C. for 2 min, followed by 30 cycles at 94° C. for 30 s alternated with an annealing temperature of 53° C. for 45 s, and extension at 72° C. for 2 min. The primers used for Dehalococcoides 16S rRNA gene were DHC730F (5′-GCG GTT TTC TAG GTT GTC-3′) and DHC1350R (5′-CAC CTT GCT GAT ATG CGG-3′).

Quantitative real-time PCR (qRT-PCR) was performed for enumeration of general 16S rRNA genes and Dehalococcoides 16S rRNA genes in culture DehaloR̂2 and Bio-Dechlor INOCULUM. The primers used for general 16S rRNA genes were Bac1055F (5′-ATG GYT GTC GTC ACCT-3′) and Bac1392R (5′-ACG GGC GGT GTG TAC-3′) (Ritalahti et al., 2006), and the probe was Bac16sTaq (FAM-CAA CGA GCG CAA CCC/3-BHQ-1/). The final volume of PCR reactions was 10 μL, and the final concentration of each chemical in a single reaction tube was: 1× RealMasterMix (Fisher Scientific, Pittsburgh, Pa.), 300 nM Probe, 300 nM each Primer, and 130 nM DNA samples. The PCR conditions were as follows: 2 min at 95° C. followed by 40 cycles of 10 s at 95° C., 20 s at 56° C. and 20 s at 68° C. The primers used for Dehalococcoides 16S rRNA genes were Dhc1200F (5′-CTG GAG CTA ATC CCC AAA GCT-3′) and Dhc1271R (5′-CAA CTT CAT GCA GGC GGG-3′), and the probe was Dhc1240Pr (FAM-TCC TCA GTT CGG ATT GCA GGC TGAA/3-BHQ-1) (He et al., 2003b). The final volume of PCR reactions was 10 μL, and the final concentration of each chemical in a single reaction tube was: 1× RealMasterMix (Fisher Scientific, Pittsburgh, Pa.), 200 nM Probe, 700 nM each Primer, and 130 nM DNA samples. The PCR conditions were as follows: 2 min at 95° C. followed by 40 cycles of 15 s at 95° C., 20 s at 58° C. and 20 s at 68° C.

Quantitative Real time PCR (qRT-PCR) was carried out in a spectrofluorimetric thermal cycler (Master cycler, epgradient S, Eppendorf). Calibration curves were performed in triplicate and a linear range of 6 orders of magnitude was obtained. Plasmids containing Dehalococcoides strain BAV1 16S rRNA genes were used as standards to construct general 16S rRNA genes and Dehalococcoides 16S rRNA genes calibration curves. The slopes of the calibration curves for general 16S rRNA genes and Dehalococcoides 16S rRNA genes were −3.46 and −3.94, and the Y intercepts were 38 and 44.2, respectively.

qRT-PCR, 16s rDNA Clone Library Construction

A General Bacteria clone library was constructed by amplifying 16S rDNA with universal bacterial primers 8F and 1525R following a protocol outline by Torres et al. We obtained 73 General Bacteria clones, and completed 17 sequences. Before analysis, we trimmed the vector sequences with SeqMan Pro software (DNASTAR) and then compared them to previously published sequences using BLAST search tool.

Bacterial primers targeting the V4 region of the 16S rRNA gene were used for pyrosequencing. The forward primer was 5′-AYT GGG YDT AAA GNG-3′, and the reverse primer was a mixture of 5′-TAC CRG GGT HTC TAA TCC-3′,5′-TAC CAG AGT ATC TAA TTC-3′,5′-CTA CDS RGG TMT CTA ATC-3′,5′-TAC NVG GGT ATC TAA TCC-3′. PCR was performed as follows: 94° C. for 2 min, 25 cycles of denaturation at 94° C. for 30 s each, 57° C. annealing for 45 s, 72° C. for 1 min extension, and a final extension at 72° C. for 2 min. Excess primer dimers and dNTPs were removed with QiaQuick spin columns. Amplicon pyrosequencing was performed by using standard 454/Roche GS-FLX protocols. After a sequencing run and base-calling, the sequences were sorted by unique tags using the 454 script (“sfffile”) to separate and group all data and then trimmed the sequences using the 454 script (“sffinfo”) for downstream analysis. The 454 reads were preprocessed to remove ambiguous and short sequences, all sequences mismatched with the PCR primers, and all sequences having less than 50 nucleotides after the proximal primer (unless they reach the distal primer). These filtering steps eliminated all the sequences having more than 1 ambiguity (N). Ribosomal Database Project Classifier 2.0 was used to assign taxonomy to pyrosequencing tags.

Results and Discussion

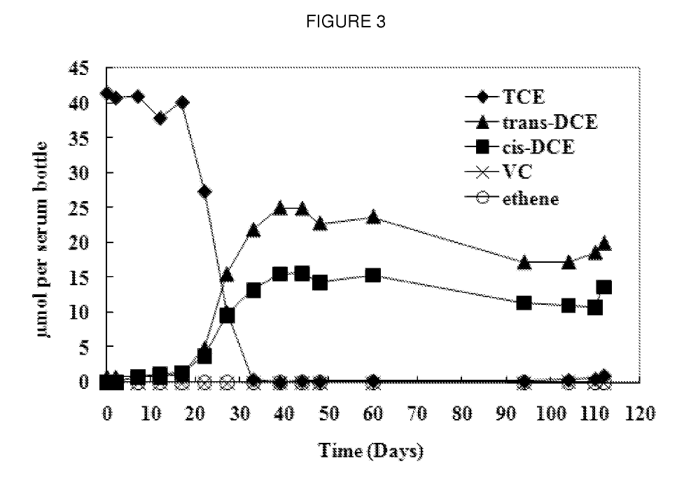

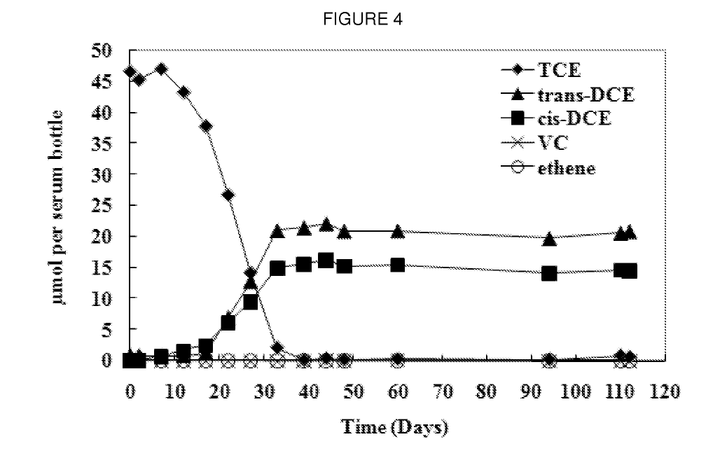

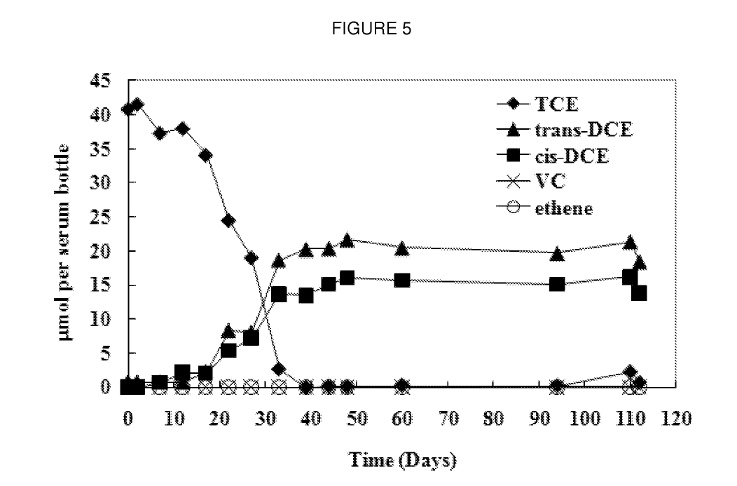

TCE Reductive Dechlorination in Sediment Microcosms. FIGS. 2 to 5 summarize the results of TCE reductive dechlorination in sediment microcosms. A lag time of 8 to 18 days was observed from the beginning of the experiment. Then, all the TCE was dechlorinated into trans-DCE and cis-DCE between the 18^{th} and the 40^{th} day, when reductive dechlorination stopped. A small amount of ethene (0.04-0.09 μmol) and vinyl chloride (0.04-0.26 μmol) were produced in the microcosms, indicating that trans-DCE and cis-DCE could be dechlorinated into VC and ethene by microorganisms perhaps through co-metabolism. A significant amount of methane was produced in all four sediment microcosms, but the concentration was not determined. The effect of the addition of 20 μg/L TCC and TCS in the microcosms on TCE reductive dechlorination can be neglected, since reductive dechlorination rates for TCE in the sediment-A and -D microcosms were the same with or without addition of TCC & TCS.

Field monitoring data proved that TCE can be abiotically dechlorinated into cis-DCE or other chlorinated ethenes in the environment (Costanza and Pennell, 2007; Darlington et al., 2008). In order to exclude the possibility that TCE degradation in our sediment microcosms was a chemical process rather than a biological one, two microcosms were set up with autoclaved sediment as abiotic controls of TCE degradation. No TCE reductive dechlorination byproducts were detected in these two microcosms in 46 days and therefore we confirmed that the reductive dechlorination of TCE in our sediment microcosms was a biological dechlorination process.

TCE in the four sediment microcosms was reductively dechlorinated to trans-DCE and cis-DCE at ratios from 1.35±0.15 to 1.67±0.15 (listed in Table 3.1), and the ratios were calculated from 8 to 10 data points over 79 to 90 days' time range. The trans-DCE/cis-DCE ratios in sediment-A microcosms with or without TCC & TCS were similar and were lower than the trans-DCE/cis-DCE ratios in sediment-D microcosms with or with TCC&TCS. The amount of trans-DCE produced in these sediment microcosms was always more than that of cis-DCE produced.

Past research on remediation of contaminated ground water and industrial sites has shown that cis-DCE is a main byproduct of PCE and TCE biodegradation and trans-DCE is not an effective energy-providing substrate for subsequent dechlorination (Griffin et al., 2004). Because of this, trans-DCE accumulates more frequently than cis-DCE at contaminated sites, and for example, it accumulates at 39% of the US Environmental Protection Agency National Priority List Sites (at least 563 of the 1,430 National Priorities List sites) compared to 10% for cis-DCE (at least 146 National Priorities List sites) (ATSDR, 2007). Some of the reductively dechlorinating microcosms or cultures can dechlorinate PCE or TCE to more trans-DCE than cis-DCE with different ratios of 1.2-1.7 in (Miller et al., 2005), 3 (Griffin et al., 2004), and 3.5 (Kittelmann and Friedrich, 2008c)), and most of these microcosms or cultures were obtained from estuarial or ocean sediment. For example, bacterium DF-1 was enriched from estuarial sediment (Miller et al., 2005; Wu et al., 2000), Griffin et al. derived the microcosms from river sediments (Griffin et al., 2004), and Kittelmann and Friedrich developed trans-DCE producing cultures from tidal flat sediments (Kittelmann and Friedrich, 2008c). This suggests that dechlorinating microbial communities in marine or river habitats are different from dechlorinating microbial communities present in groundwater and soil systems. The TCE dechlorination process and the ratio of trans-DCE/cis-DCE in our sediment microcosms is similar to those reported for bacterium DF-1 (Miller et al., 2005); this is reasonable since DF-1 was isolated from a similar sediment source, an estuarial sediment.

TABLE 3
Ratios of trans-DCE/cis-DCE in four sediment microcosms.

| | | Number of | |
|---|---|---|---|
| Microcosms | trans-DCE/ | data points | |
| Name | cis-DCE ratio | (n) | Time range |
| Sediment-D | 1.67 ± 0.15 | 9 | 85 days, 27 to 112 |
| | | | days |
| Sediment-D | 1.58 ± 0.14 | 10 | 90 days, 22 to 112 |
| with TCC&TCS | | | days |
| Sediment-A | 1.38 ± 0.04 | 9 | 85 days, 27 to 112 |
| | | | days |
| Sediment-A | 1.35 ± 0.15 | 8 | 79 days, 33 to 112 |
| with TCC&TCS | | | days |

TCE Reductive Dechlorination in Sediment Microcosms Constructed to Suppress Methanogenesis by Addition of BES. There are three possible explanations for partial TCE reductive dechlorination to trans-DCE and cis-DCE in sediment-A and -D microcosms: 1) microorganisms, such as Dehalococcoides, that can dechlorinate trans-DCE and cis-DCE into less or non-chlorinated ethenes were not present in the sediment; so, regardless of external conditions provided, complete reductive dechlorination of TCE could never be achieved, 2) methanogens competed with dechlorinating microorganisms for electron donors and or carbon sources, which inhibited reductive dechlorination and blocked TCE dechlorination at the trans-DCE and cis-DCE steps, and 3) the sediment used in the research contained a high concentration of TCC and TCS inhibited complete TCE reductive dechlorination.

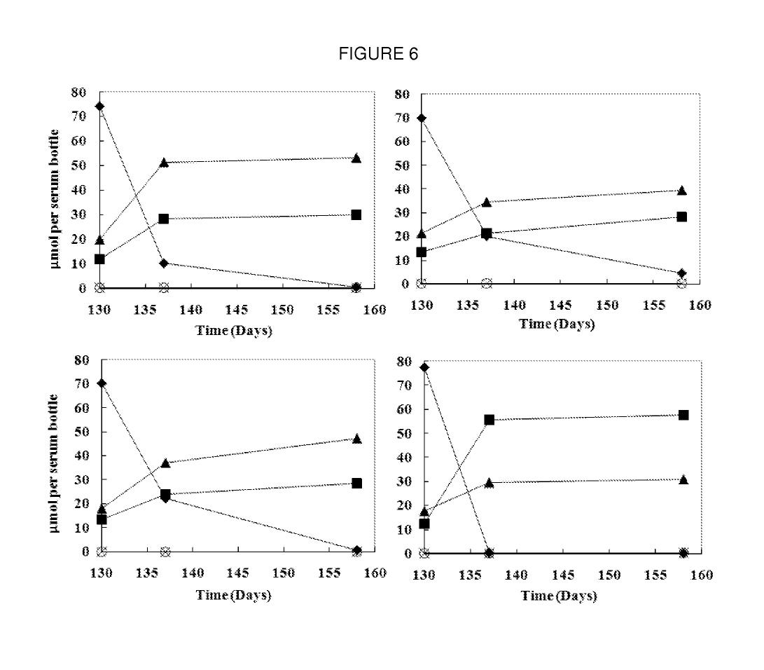

BES (2-bromoethanesulfonate) is an efficient inhibitor for the metabolism of methanogens in methanogenetic cultures because it is a structural analog and competitive inhibitor of coenzyme M, which is only found in methanogens (Dimarco et al., 1990; Sparling and Daniels, 1987). Different concentrations of BES have been used by researchers. Löffler et al. suggested the dosage of 2 mM BES to inhibit methanogens in reductively dechlorinating cultures (Löffler et al., 1997), Freedman and Gossett used 5 mM BES for the same purpose (Freedman and Gossett, 1989), Roy et al. used 20 mM for early initiation of methane production in anoxic rice soil (Roy et al., 1997), Metje et al. used 40 mM to completely inhibit methanogenesis including acetoclastic methanogenesis (Metje and Frenzel, 2005), and some other researchers used 25 mM BES to effectively inhibit methanogens (Lomans et al., 1997; Lyimo et al., 2002; Oremland and Capone, 1988; Sipma et al., 2003). In this study, in order to determine if the cause for incomplete reductive dechlorination was competition for electron donor between methanogens and TCE dechlorinating microorganisms in the microcosms, I added 25 mM BES, 70 μmol of TCE, and 5 mM lactate into two sediment microcosms, sediment-A and sediment-D with TCC and TCS. In two other sediment microcosms, I added 70 μmol of TCE and 5 mM lactate without BES. The results are shown in FIG. 6.

According to FIG. 6, the TCE dechlorination rate in all sediment microcosms increased compared to the previous dechlorination rates showed in FIGS. 2 to 5. More than 70% of the 70 μmol TCE was reductively dechlorinated to trans-DCE and cis-DCE in 7 days and no methane was produced in the two sediment microcosms amended with BES; however, the reductive dechlorination process still stopped at trans-DCE and cis-DCE and the concentration and production of VC and ethene were stable and negligible. Only 0.03 to 0.13 μmol ethene and 0.09 to 0.19 μmol VC were produced at the beginning of the experiment.

The addition of 25 mM BES did not improve the TCE dechlorination in sediment microcosms; however, it changed the trans-DCE/cis-DCE ratios in TCE reductive dechlorination, as shown in Table 4. In sediment-A microcosm with BES, cis-DCE became the main dechlorination product, and the ratio of trans-DCE/cis-DCE decreased from 1.40 to 0.53; and in sediment D microcosm with BES, TCC and TCS, the trans-DCE/cis-DCE decreased from 1.60 to 1.40, indicating that the amount of BES added had some inhibition on reductive dechlorination of TCE to trans-DCE. On the other hand, the trans-DCE/cis-DCE ratio in the sediment microcosms amended only with 70 μmol TCE and lactate (no BES) increased from 1.35 to 1.66 and from 1.69 to 1.77 respectively in sediment-A microcosm with TCC & TCS and in sediment-D microcosm.

This experiment showed that 25 mM BES exerted some inhibition on TCE reductive dechlorination which is consistent with past research (Fathepure and Boyd, 1988; Freedman and Gossett, 1989; Löffler et al., 1997), and the results also show that: 1) 25 mM BES may inhibit the dechlorination of TCE into trans-DCE; 2) it is likely that methanogens are involved in TCE dechlorination to trans-DCE, and 3) this does not exclude the possibility that other TCE declorinating microorganisms can be inhibited by BES. Further research is needed to explore the reason for change in ratio of trans-DCE/cis-DCE after 25 mM BES was added into the microcosms.

These results suggest that methanogens and insufficiency of electron donor and acceptor are not the reason for partial TCE reductive dechlorination to trans-DCE. Therefore, I hypothesize that either microorganisms capable of reductively dechlorinating TCE to ethene are not present in the sediment or inhibition of TCC and TCS results in incomplete TCE reductive dechlorination.

TABLE 4
Ratios of trans-DCE/cis-DCE in four sediment
microcosms after adding BES.

| Microcosms Name | trans-DCE/cis-DCE ratio | Time range |
|---|---|---|
| Sediment-A with BES | Decrease from | 130 to 158 days |
| | 1.40 to 0.53 | |
| Sediment-A with | Increase from | 130 to 158 days |
| TCC & TCS | 1.35 to 1.66 | |
| Sediment-D | Increase from | 130 to 158 days |
| | 1.69 to 1.77 | |
| Sediment-D with BES, | Decrease from | 130 to 158 days |
| TCC & TCS | 1.60 to 1.40 | |

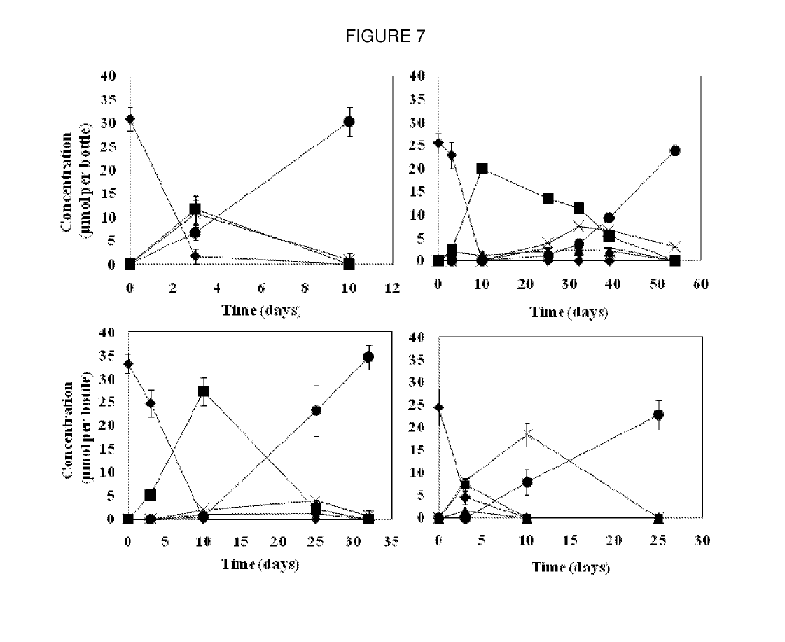

TCE Reductive Dechlorination in Sediment-Free Cultures. Complete biodegradation of TCE was not achieved in the sediment microcosms in 7 months of enrichment. At that point 12 sediment-free cultures were generated from the 4 sediment microcosms (FIG. 1). Surprisingly, TCE was rapidly dechlorinated into ethene in these sediment-free cultures (FIG. 7), especially in the DehaloR̂2 cultures where complete dechlorination to ethene was observed at the fastest rates: 32 mmol TCE (33.6 mg/L) were completely dechlorinated into ethene in 10 days (FIG. 7). It took about 32 days, 25 days and 54 days for the complete reductive dechlorination of TCE in SCAB cultures, SCAT cultures, and SCDBT cultures respectively (FIG. 7). Small amounts of trans-DCE and 1,1-DCE, about 1 to 3 μmol per serum bottle, were produced in the TCE reductive dechlorination process by SCAB, SCAT, and SCDBT cultures, but cis-DCE was the main byproduct.

According to the maximum utilization rate and maximum formation rate of TCE, cis-DCE, VC and ethene in Table 5, different sediment-free cultures presented different TCE dechlorination rates. The rates ranked from faster to slowest were as follow: DehaloR̂2>SCAT>SCAB>SCDBT. The DehaloR̂2 cultures had the highest TCE utilization rate and ethene formation rate, which were 9.73 μmol/day and 3.36 μmol/day, respectively. Conversely, the SCDBT cultures had the lowest TCE utilization rate and ethene formation rate which were 3.26 μmol/day and 0.96 μmol/day respectively, and complete dechlorination of TCE took the longest time, 54 days. Thus, TCC, TCS and BES clearly have an effect on TCE dechlorination. They slow down TCE dechlorination rates.

Complete dechlorination of TCE in sediment-free cultures proved that microorganisms that can completely dechlorinated TCE into ethene were present in the sediment, and the reason for partial reductive dechlorination of TCE in sediment microcosms remained an open question for which inhibition by TCC and TCS could be a possible answer.

TABLE 5
Dechlorination rates in sediment-free cultures. (Rates are based
on the average concentration of triplicate samples.)

| | Maximum | | | Maximum | | | |
|---|---|---|---|---|---|---|---|
| | utilization rate | | | formation rate | | | Days for |
| Sediment- | (μmol vial⁻¹ day⁻¹) | | | (μmol vial⁻¹ day⁻¹) | | | complete |
| free | | cis- | | cis- | | | dechlori- |
| cultures | TCE | DCE | VC | DCE | VC | Ethene | nation |
| DehaloR\hat{ }2 | 9.73 | 1.67 | 1.42 | 3.89 | 3.59 | 3.36 | 10 |
| SCDBT | 3.26 | 2.15 | 0.23 | 2.49 | 0.50 | 0.96 | 54 |
| SCAB | 3.54 | 1.69 | 0.13 | 3.18 | 0.46 | 1.62 | 32 |
| SCAT | 6.66 | 1.05 | 1.23 | 2.45 | 2.70 | 0.99 | 25 |

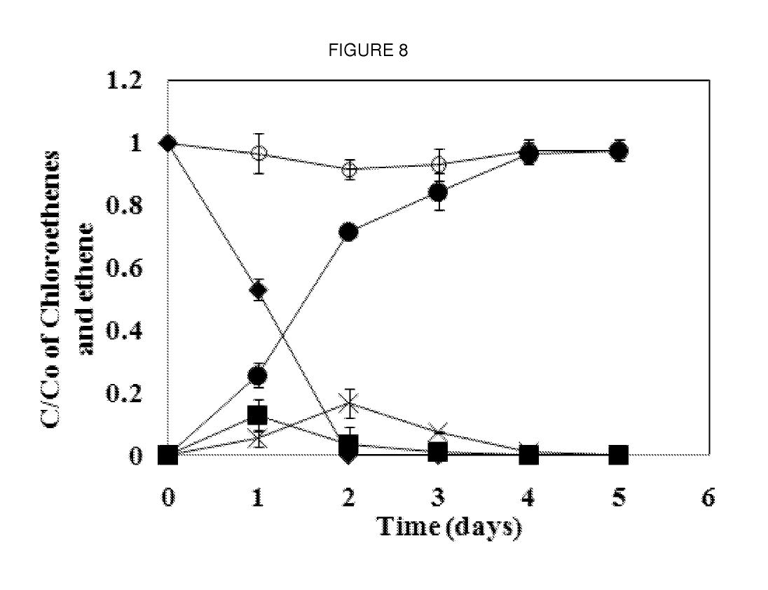

In order to obtain dynamic data of TCE reductive dechlorination in DehaloR̂2 cultures, the triplicate cultures were spiked with 40 μmol TCE (42.0 mg/L) and 5 mM lactate and the concentrations of TCE and its dechlorination products were tested every day. The TCE dechlorination rate was greater than when TCE was initially added into DehaloR̂2 cultures. Complete reductive dechlorination of TCE to ethene took only 5 days (FIG. 8). All TCE was dechlorinated into VC and ethene in 2 days, and there was no apparent cis-DCE or VC accumulation in the reductive dechlorination process. As shown in FIG. 8, there was no significant peak of cis-DCE or VC, which is unusual in TCE dechlorination process.

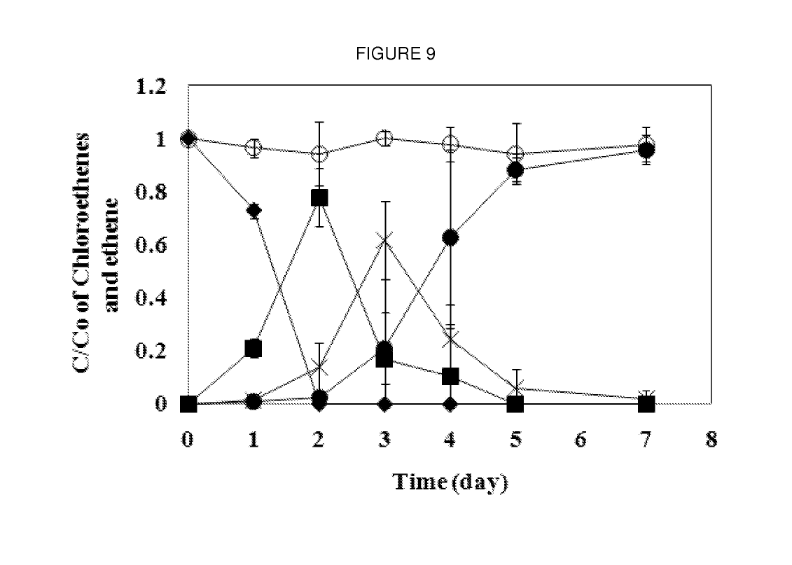

TCE reductive dechlorination was extremely fast in the DehaloR̂2 cultures, making it challenging to measure intermediates of the dechlorination process, unless samples were taken on a more frequent basis. In order to measure intermediates in the process, 10% (v/v) of DehaloR̂2 culture was transferred into three bottles of fresh anaerobic media to generate the second generation of DehaloR̂2 cultures. All of the TCE was dechlorinated to less chlorinated ethenes in 2 days, and at the 3^{rd} day VC became the dominant byproduct. On the 4^{th} day, ethene was the main dechlorination product. It took about 7 days for generation II DehaloR̂2 cultures to completely dechlorinate 26 μmol TCE (FIG. 9) which is much faster than the popular TCE/PCE reductive dechlorinating culture, Bio-Dechlor INOCULUM (BDI). Under the same conditions it took BDI 20 days to dechlorinate 22.5 μmol TCE (23.6 mg/L) into less chlorinated ethenes, and the complete dechlorination of 22.5 μmol TCE to ethene took 35 days (Sung, 2005a).

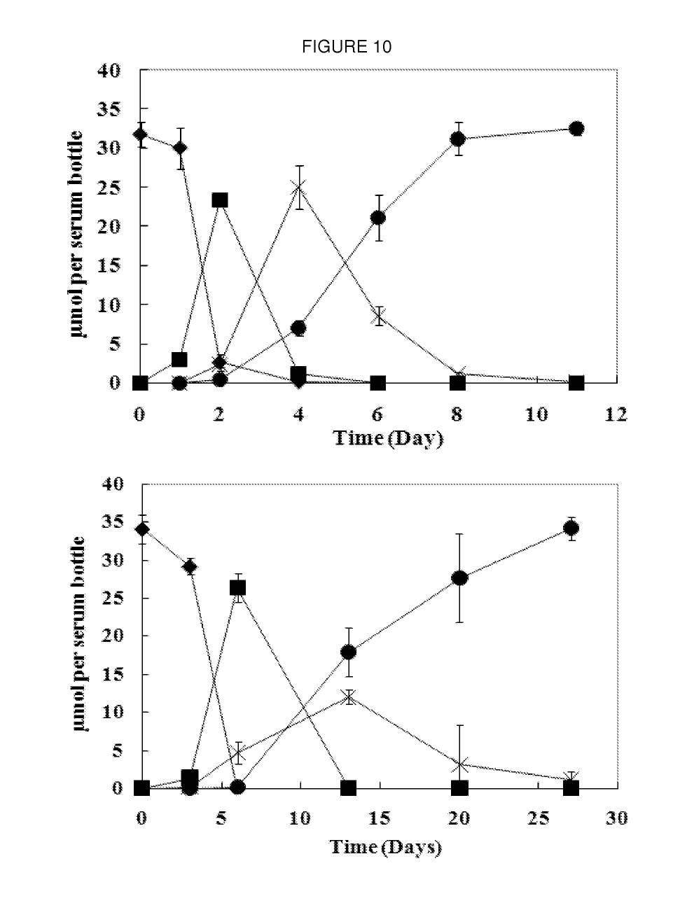

In order to compare the performance of DehaloR̂2 culture and BDI culture, TCE reductive dechlorination was tested in generation III DehaloR̂2 cultures and the BDI cultures cultivated in our laboratory (FIG. 10). Generation III DehaloR̂2 cultures took about 11 days to dechlorinate 32 μmol TCE, and for BDI cultures, they took about 27 days.

According to the qRT-PCR data, the maximum Dehalococcoides 16S rRNA gene copies in DehaloR̂2 cultures and BDI cultures were 2.6×10⁷ and 2.6×10⁸ gene copies/ml, respectively. The maximum general bacteria 16S rRNA gene copies in DehaloR̂2 cultures and BDI cultures were 1.8×10⁹ and 2.3×10⁸ gene copies/ml, respectively. Therefore, the biomass in DehaloR̂2 culture was about 7.9 times of that in BDI cultures, but Dehalococcoides in DehaloR̂2 cultures was only 10% of that in BDI cultures.

The dechlorination rates based on the maximum Dehalococcoides 16S rRNA gene copies are shown in Table 6. The dechlorination rates in DehaloR̂2 cultures were much higher than that in BDI cultures, which was consistant with the rapid TCE dechlorination process observed in DehaloR̂2 cultures. However, the dechlorination rates based on the maximum general bacteria 16S rRNA gene copies in DehaloR̂2 cultures were lower than that in BDI cultures. The reason is that based on our qRT-PCR analysis more than 90% of general bacteria in BDI cultures were Dehalococcoides, but only 30% of general bacteria in DehaloR̂2 cultures were Dehalococcoides. DehaloR̂2 cultures have more biomass but less Dehalococcoides. The better performance of DehaloR̂2 cultures partly proves that DehaloR̂2 cultures are new and have a more efficient TCE dechlorinating microbial community, and the diversity in dechlorinating cultures is important.

TABLE 6
Dechlorination rates based the maximum Dehalococcoides 16S rRNA
gene copies in DehaloR\hat{ }2 cultures and BDI cultures.

| | Maximum utilization rate | | | Maximum formation rate | | |
|---|---|---|---|---|---|---|
| | μmol/(10¹¹ Gene copies · day) | | | μmol/(10¹¹ Gene copies · day) | | |
| Cultures | TCE | cis-DCE | VC | cis-DCE | VC | Ethene |
| DehaloR\hat{ }2 | 429.6 ± 18.9 | 173.8 ± 7.7 | 129.0 ± 5.7 | 319.3 ± 14.1 | 177.8 ± 7.8 | 111.3 ± 4.9 |
| BDI | 37.2 ± 1.1 | 14.4 ± 0.4 | 4.9 ± 0.2 | 31.9 ± 1.0 | 5.9 ± 0.2 | 9.8 ± 0.3 |

TABLE 7
Dechlorination rates based general bacteria 16S rRNA gene copies
in DehaloR\hat{ }2 cultures and BDI cultures.

| | Maximum utilization rate | | | Maximum formation rate | | |
|---|---|---|---|---|---|---|
| | μmol/(10¹¹ Gene copies · day) | | | μmol/(10¹¹ Gene copies · day) | | |
| Cultures | TCE | cis-DCE | VC | cis-DCE | VC | Ethene |
| DehaloR\hat{ }2 | 14.8 ± 0.8 | 6.0 ± 0.3 | 4.5 ± 0.2 | 11.0 ± 0.6 | 6.1 ± 0.3 | 3.8 ± 0.2 |
| BDI | 41.5 ± 5.4 | 16.1 ± 2.1 | 5.4 ± 0.7 | 35.6 ± 4.6 | 6.6 ± 0.9 | 10.9 ± 1.4 |

PCE Reductive Dechlorination in DehaloR²Cultures. Because DehaloR̂2 cultures can dechlorinate TCE quickly, it is important to know if this culture has a wide range of biodegradation capability so PCE dechlorination was also tested in the DehaloR̂2 cultures.

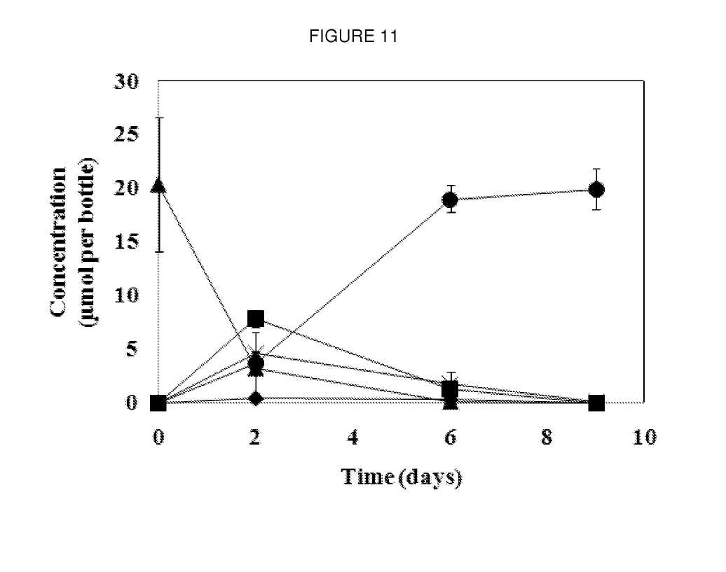

The results showed that in DehaloR² cultures PCE can be dechlorinated to ethene (FIG. 11). VC and ethene were produced at the beginning of PCE dechlorination, and no TCE accumulation was observed because this culture was well acclimated with TCE for a long time. At the 6^{th} day, almost 80% of PCE was dechlorinated into ethene, and in 9 days 20 μmol PCE (22.9 mg/L) were completely dechlorinated into ethene.

PCR and Microbial Community Analysis. DNA was extracted from DehaloR̂2 culture, sediment D, SCAT culture and sediment A. General bacteria PCR successfully amplified 16S rRNA genes of all the DNA samples, but Dehalococcoides amplicons generated with Dehalococcoides targeted primers were only detected in DNA extracted from the sediment-free cultures. The positive control was amplified successfully. DNA extracted from sediment A and sediment D was not amplified by PCR targeting Dehalococcoides and nested PCR using first primers targeting the general bacteria 16S rRNA genes followed by PCR targeting Dehalococcoides 16S rRNA genes, indicating that Dehalococcoides were either not present or present at extremely low concentrations in the sediment samples.

The total number of tags obtained by pyrosequensing for the DehaloR̂2 culture, sediment D, SCAT culture and sediment A samples was 4781, 7109, 6593 and 5308, respectively. The structure of the microbial community derived from pyrosequensing data for DehaloR̂2 culture, sediment D, SCAT culture and sediment A is presented in FIG. 11. Proteobacteria were the dominant sequences detected in sediment A and D, occupying 81.4% and 72.0% of all sequences, which was similar in the tidal flat sediments (Kittelmann and Friedrich, 2008c). However, in SCAT and DehaloR̂2 cultures, Proteobacterial sequences decreased to 1.0% and 3.4%, respectively. Firmicutes became the major sequences detected in the sediment-free cultures, present at 73.1% and 66.9% of the sequences from SCAT and DehaloR̂2, instead of 1.1% and 1.6% in sediment A and D. Since most Firmicutes are fermenters, adding abundant lactate in the sediment-free cultures may be the possible factor that biased the microbial community structure in the cultures.

Dehalococcoides were not detected in sediment samples, but in the microbial communities of SCA and DehaloR̂2 Dehalococcoides sequences were 0.2% and 0.4% of the overall sequences gathered, respectively. Chloroflexi sequences, the phylum to which Dehalococcoides belongs, also increased in the sediment-free cultures.

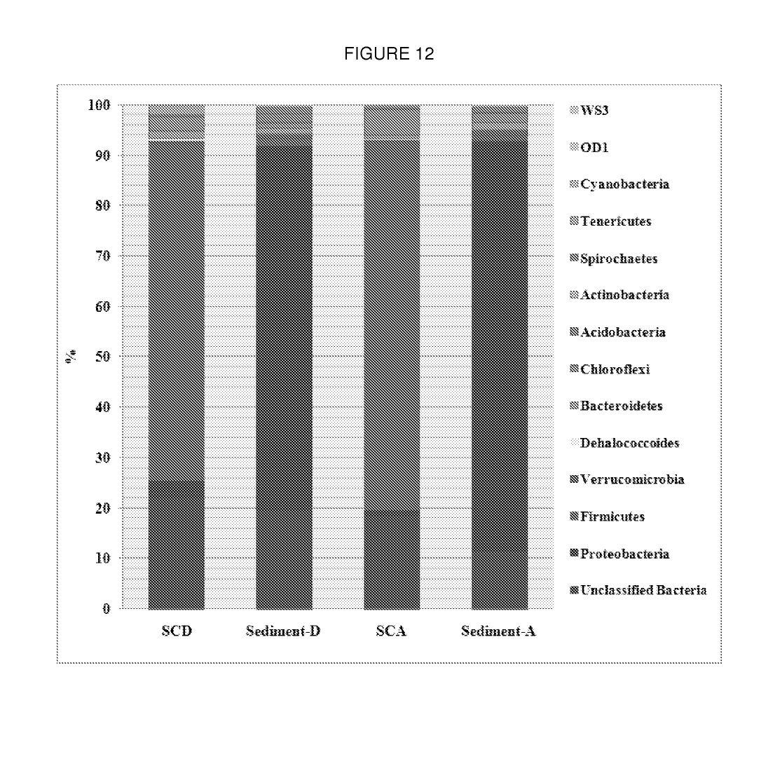

The classes of Proteobacteria detected in sediments and cultures are presented in FIG. 12. Deltaproteobacteria was the main class of Proteobacteria sequences detected in sediment-free cultures, but in the sediment samples, Betaproteobacteria and Epsilonproteobacteria were the major classes of Proteobacterial sequences detected. Betaproteobacteria and Deltaproteobacteria may be involved in the incomplete dechlorination of TCE in the sediment microcosms, since recently they were detected by RNA-based isotope probing in a PCE dehalorespirating microcosm community (Kittelmann and Friedrich, 2008a). Deltaproteobacteria contains Geobacter genus and Desulfuromonas genus, and these two genuses are putative TCE dechlorinators (Deweerd et al., 1990; Sung et al., 2006a; Sung et al., 2003). Hence, the communities with more Deltaproteobacteria may have a stronger reductive dechlorinating capability. The percentages of Geobacter in Proteobacteria phylum in DehaloR̂2, sediment D, SCAT and sediment A were 3.05%, 0.57%, 2.90% and 0.14% respectively. Geobacter lovleyi can dechlorinate PCE/TCE into cis-DCE (Sung et al., 2006a). Therefore, Geobacter are vigorously growing in the sediment-free cultures, becoming one of the possible reasons that cis-DCE instead of trans-DCE became one of the main byproducts of TCE reductive dechlorination. Additionally, Geobacter is a dechlorinator and actively living in KB-1 cultures fed with PCE/TCE (Duhamel and Edwards, 2006), which is consistent with the results presented herein. Trichlorobacter sequences were only present in DehaloR̂2 culture, which was also observed by Dennis et al. in an anaerobic microbial community capable of degrading saturation levels of PCE (Dennis et al., 2003).

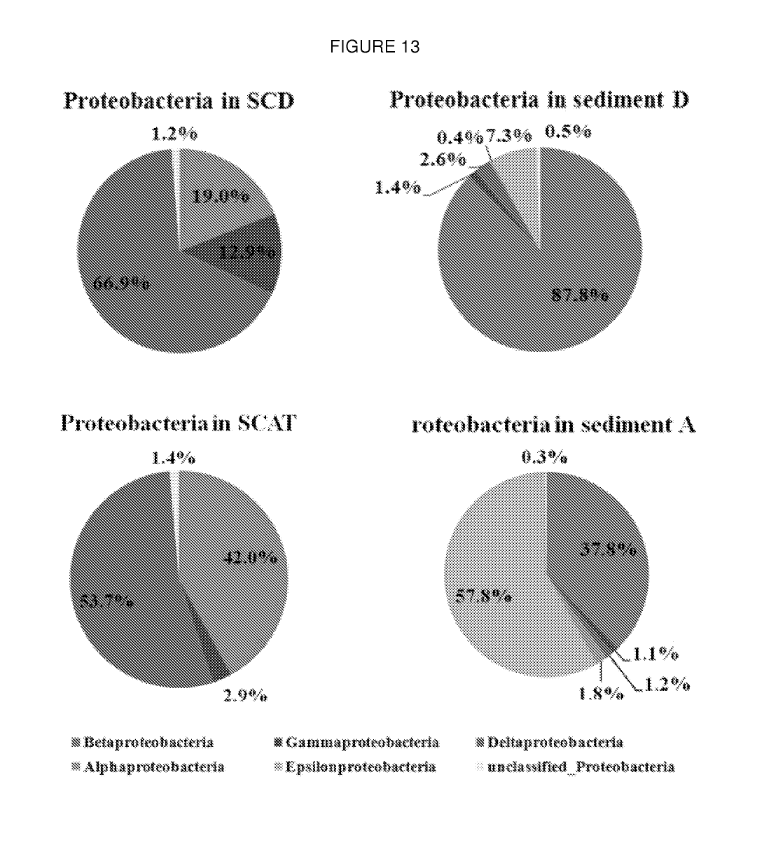

Firmicutes sequences were the dominant phylum in sediment-free cultures. The percentages of different genus of Firmicutes sequences gathered from sediment and culture samples are presented in FIG. 13. One significant change is the Acetobacterium percentage in Firmicutes sequences, which increased from 7.9% in sediment D to 50.3% in DehaloR̂2, and from 0% in the sediment A to 9.8% in SCAT. Moreover, Clostridium genus in Firmicutes phylum, another TCE/PCE dechlorinator, presented in the DehaloR̂2 culture, may also contribute to the reductive dechlorination capability of DehaloR̂2.

Acetobacterium is a genus of homoacetogens, it can produce acetate from carbon dioxide and hydrogen (Diekert, 1990). Dehalococcoides cannot use inorganic carbon sources, and the literature reports that they need acetate as a carbon source to support their growth (He et al., 2005b). On the other hand, some dechlorinators, such as Desulfuromonas can also use acetate as electron donor for TCE reductive dechlorination (Deweerd et al., 1990; Sung et al., 2003). Furthermore, acetogenesis related to Co(I) corrinoids, and Co(I) corrinoids can reduce halogenated organic compounds (clod et al., 1997; Holliger et al., 1992; Stupperich, 1993); therefore, acetogens are able to cometabolically dechlorinate PCE, TCE and 1,2-dichloroethane (1,2-DCA) (Wild et al., 1995). Acetobacterium was isolated and the pure culture of Acetobacterium can dechlorinate 1,2-DCA to ethene with a maximum dechlorination rate of 2 nmol Cl⁻/(min·mg of protein) (De Wildeman et al., 2003). Thus, Acetobacterium appears to play an extremely important role in reductive dechlorinating communities.

Although Dehalococcoides sequences were only a small part of the overall sequences gathered from sediment-free cultures, other putative dechlorinating bacteria, such as Acetobacterium, Geobacter, Trichlorobacter and Clostridium, increased in the microbial communities following enrichment in our laboratory. Perhaps they constitute an efficient reductive dechlorinating team, because of this culture DehaloR̂2 can quickly and completely dechlorinate TCE into ethene.

### SUMMARY

Four sediment microcosms were set up from the sediment core A and sediment core D, and TCE reductive dechlorination was tested in these sediment microcosms. There was an 8 to 18 days lag time, and then TCE was quickly dechlorinated into trans-DCE and cis-DCE from the 18^{th} day to the 40^{th} day; however, the reductive dechlorination process stopped at this point and the reason was not clear. The trans-DCE/cis-DCE ratios in the four sediment microcosms were from 1.35±0.15 to 1.67±0.15.

After adding 25 mM BES and additional TCE, TCE dechlorination in sediment microcosms still stopped at trans-DCE and cis-DCE; however, the trans-DCE/cis-DCE ratios in TCE reductive dechlorination decreased, which indicated that 25 mM BES also inhibited the reductive dechlorination from TCE to trans-DCE. Furthermore, in the two microcosms amended with 70 μmol TCE and 5 mM lactate, the trans-DCE/cis-DCE ratios increased which implied that adding 70 μmol TCE may be helpful to enrich reductive dechlorinating microorganisms which dechlorinated TCE into cis-DCE.

Sediment-free cultures showed rapid TCE dechlorination into ethene, especially in the DehaloR² cultures, TCE was completely dechlorinated into ethene in 10 days. Additionally, in SCAB cultures, SCAT cultures, and SCDBT cultures, it took about 32 days, 25 days, and 54 days respectively for the complete TCE reductive dechlorination. Thus, the sediment-free cultures presented a different TCE dechlorination rates: DehaloR̂2>SCAT>SCAB>SCDBT.

The maximum utilization rates and maximum formation rates of TCE, cis-DCE, VC and ethene in sediment-free cultures proved that TCC, TCS and BES inhibited TCE dechlorination by slowing down the TCE dechlorination rate.

Although complete dechlorination to ethene was not observed in microcosms, we confirmed with our sediment-free cultures that there are microorganisms present in the sediment able to reductively dechlorinate TCE to ethene. The TCE dechlorination results with the addition of BES, TCE, and lactate showed that even with methanogens inhibited and electron donor and carbon source provided TCE dechlorination in microcosms was still incomplete. Therefore, the high concentration of TCC or TCS in the sediment may inhibit TCE reductive dechlorination, and this could be an ultimate reason for incomplete TCE biodegradation in sediment microcosms.

PCR and pyrosequensing data showed that Dehalococcoides sequences were not detected in the sediment D and A samples; however, in the DehaloR̂2 and SCAT samples, DNA was amplified with PCR primers targeting Dehalococcoides. According to pyrosequencing data, Dehalococcoides sequences were 0.4% and 0.2% of the analyzed microbial community sequences in DehaloR̂2 and SCAT respectively. Proteobacteria phylum is dominant in sediment microbial community, and in the sediment-free cultures the dominant bacteria shifted to Firmicutes phylum. In DehaloR̂2 and SCAT, although Dehalococcoides was a small group of bacteria, lots of other putative dechlorinating bacteria, such as Acetobacterium, Geobacter, Trichlorobacter and Clostridium sequences, increased and were present in the microbial communities, these microorganisms could help and collaborate with Dehalococcoides to achieve complete reductive dechlorination of PCE or TCE to ethene.

The DehaloR̂2 culture is a valuable microbial source for TCE or PCE bioremediation, because more chloroethenes dechlorinators are present in the microbial community and the dechlorination of TCE and PCE can proceed much faster than other available cultures. For example, BDI culture takes about 35 day to dechlorinate 22.5 μmol TCE per serum bottle into ethene (Sung, 2005a), while the DehaloR̂2 culture only takes about 5 days to dechlorinate 40 μmol TCE per serum bottle under the same conditions.

Further research is needed to explore the effect of BES on reductive dechlorination from TCE to trans-DCE, assess possible reductive dechlorination of TCC and TCS by the sediment-free culture, isolate microbial species with reductive dechlorination capability in the sediment-free cultures, and seek new high efficient dechlorinating bacteria like Dehalococcoides.

### Example 2

### Effects of TCC and TCS on TCE Reductive Dechlorination

In example 1 it was observed that reductive dechlorination of TCE in microcosms established with sediments from the CB stopped at trans-DCE or cis-DCE, and it was hypothesized that one of the causes could be the presence of TCC and TCS in the sediments. To asses that possibility, the following example explores the inhibitory effects of TCC and TCS on two TCE dechlorinating cultures.

### Materials and Methods

Chemicals. PCE and TCE were purchased from Sigma-Aldrich Co. (St. Louis, Mo.). cis-DCE, trans-DCE and 1,1-DCE were purchased from Supelco Co. (Bellefonte, Pa.). Gaseous VC was obtained from Fluka Chemical Corp. (Ronkinkoma, N.Y.), and ethene and ethane were purchased from Scott Specialty Gases (Durham, N.C.). TCC was obtained from Sigma-Aldrich Co. (St. Louis, Mo.) with 99% purity and TCS was purchased from TCI America (Portland, Oreg.) with 96% purity.

Cultures. Two cultures were used in this study: 1) Bio-Dechlor INOCULUM, which was obtained from Dr. Frank Loffler's laboratory at Georgia Tech; 2) DehaloR̂2 culture, developed from a sediment microcosm from the Chesapeake Bay “sediment core-D” with three-times continuous transfers described in Example 1.

TCC and TCS Exposure. For the TCC and TCS test, 1 ml ATCC mixed vitamin solution (Catalog No. MD-VS), 0.25 ml 20 mg/L vitamin B₁₂ solution and 1 ml 500 mM lactate solution were filtered with a 0.2 μm sterile filter and added to 92.5 ml sterile anaerobic media in 160 ml serum bottles, and then 5 ml of culture was transferred into this fresh media. Lactate served as electron donor and carbon source. Fifty μl TCE stock solutions (0.7 mM in methanol) were injected into the media with 500-μl Hamilton air-tight syringe (Reno, Nev.), and the final TCE concentration was 35 μmol per serum bottle. Ten μl TCC or TCS stock solutions in methanol or pure methanol was injected with 10 μl Hamilton air-tight syringe (Reno, Nev.) into the serum bottle. Three concentration levels of TCC or TCS stock solutions were made and listed in Table 8 to make the three final concentration levels of TCC or TCS. The middle concentration level of TCC and TCS in cultures was set according to the solubility of TCC and TCS at 20° C. The highest concentration of TCC and TCS is 10 times the solubility of TCC and TCS at 20° C., and the lowest concentration of TCC and TCS is 10% the solubility of TCC and TCS at 20° C.

The total aqueous volume in 160-ml serum bottle was 100 ml. Four BDI and four DehaloR̂2 cultures were generated for each concentration of TCC or TCS, and three of them were used for TCE dechlorination analysis and the other one was used for DNA extractions and molecular analysis. The error bars in the figures are the standard deviations for three independent cultures under the same conditions.

TABLE 8
TCC and TCS concentrations in methanol stock solutions and cultures.

| | | |
|---|---|---|
| | | Final concentration |
| Chemical | Stock solution | in cultures |

| | | | | |
|---|---|---|---|---|
| TCC | 15 mM | (about 5 g/L) | 1.5 μM | (500 μ/L) |

| | |
|---|---|
| 1.5 mM | 0.15 μM |
| 0.15 mM | 0.015 μM |

| | | | | |
|---|---|---|---|---|
| TCS | 350 mM | (about 100 g/L) | 35 μM | (10 mg/L) |

| | |
|---|---|
| 35 mM | 3.5 μM |
| 3.5 mM | 0.35 μM |

The TCC and TCS tests on BDI culture were conducted first, and the results showed that the lowest concentration of TCC and TCS had no influence on TCE reductive dechlorination. The TCC and TCS tests on DehaloR̂2 culture were only dosed with the highest and middle concentrations of TCC and TCS, which are 1.5 μM TCC, 0.15 μM TCC, 35 μM TCS and 3.5 μM TCS, respectively.

All cultures were stored in the dark and incubated at 30° C.

Analytical Methods. The concentrations of TCE and their reductive-dechlorinating byproducts, 1,1-DCE, cis-DCE, trans-DCE, VC, and ethene were quantified by gas chromatography with a flame ionization detector (GC-FID).

200 μl headspace gas samples were withdrawn from 160 ml serum bottle with 500 μl gas-tight syringes (Hamilton Company, Reno, Nev.) and analyzed by a Shimadzu GC-2010 (Columbia, Md.) with an Rt™-QSPLOT capillary column (30 m×0.32 mm×10 μm, Restek, Bellefonte, Pa.) and a flame ionization detector (FID). The initial oven temperature was 110° C. and was held constant for 5 min. Then the temperature was raised with a gradient of 10° C./min to 150° C., a second gradient at 20° C./min to 200° C., and a third gradient of 5° C./min to 220° C. At the end of this gradient, the oven temperature was held constant at 220° C. for 5 min. The temperature of FID and injector were 240° C. Ultra high purity helium was the carrier gas for the GC. Ultra high purity hydrogen and zero grade air were used for the FID.

Calibration curves for chlorinated compounds were determined based on the known mass of PCE, TCE, 1,1-DCE, cis-DCE and trans-DCE added to 160 ml serum bottles containing 100 ml of distilled-deionized water and equilibrated at 25° C. overnight. The contents were then analyzed using the Shimadzu GC-FID. Different volumes of 100 ppm and 1000 ppm VC and ethene gas were injected directly into GC using the 500 μl airtight syringe to make their calibration curves. The aqueous concentrations of PCE, TCE and their reaction products were calculated by using reported Henry's constants on the EPA website (Washington and Weaver, 2006)

DNA Extraction and Quantitative Real-Time PCR. The BDI cultures and DehaloR² cultures were shaken vigorously first and then 10 ml of the mixed liquid was removed with a sterile syringe and dispensed into 10 1.5 ml micro centrifuge tubes. These micro centrifuge tubes were then centrifuged at 13,000 rpm for 15 min in order to make a pellet, and the pellet was used for DNA extraction. Qiagen DNeasy Blood & Tissue Kit with modifications to enhance lysis was used for DNA isolation from cultures. DNA samples were stored at −20° C.

Quantitative real-time PCR (qRT-PCR) was performed for enumeration of general 16S rRNA genes and Dehalococcoides 16S rRNA genes in BDI culture without TCC&TCS, BDI culture with 1.5 μM TCC, BDI culture with 35 μM TCS, D DehaloR̂2 culture without TCC&TCS, DehaloR² culture with 1.5 μM TCC and DehaloR̂2² culture with 35 μM TCS.

The primers used for general 16S rRNA genes were Bac1055F (5′-ATGGYTGTCGTCAGCT-3′) and Bac1392R (5′-ACGGGCGGTGTGTAC-3′) (Ritalahti et al., 2006), and the probe was Bac16sTaq (FAM-CAACGAGCGCAACCC/3-BHQ-1/). The final volume of PCR reactions was 10 μL, and the final concentration of each chemical in a single reaction tube was: 1× RealMasterMix (Fisher Scientific, Pittsburgh, Pa.), 300 nM Probe, 300 nM each Primer, and 130 nM DNA samples. The PCR conditions were as follows: 2 min at 95° C. followed by 40 cycles of 10 s at 95° C., 20 s at 56° C. and 20 s at 68° C.

The primers used for Dehalococcoides 16S rRNA genes were Dhc1200F (5′-CTGGAGCTAATCCCCAAAGCT-3′) and Dhc1271R (5′-CAACTTCATGCAGGCGGG-3′), and the probe was Dhc1240Pr (FAM-TCCTCAGTTCGGATTGCAGGCTGAA/3-BHQ-1) (He et al., 2003b). The final volume of PCR reactions was 10 μL, and the final concentration of each chemical in a single reaction tube was: 1× RealMasterMix (Fisher Scientific, Pittsburgh, Pa.), 200 nM Probe, 700 nM each Primer, and 130 nM DNA samples. The PCR conditions were as follows: 2 min at 95° C. followed by 40 cycles of 15 s at 95° C., 20 s at 58° C. and 20 s at 68° C.

The qRT-PCR was carried out in a spectrofluorimetric thermal cycler (Master cycler, epgradient S, Eppendorf). Calibration curves were performed in triplicate and a linear range of 6 orders of magnitude was obtained. Plasmids containing Dehalococcoides strain BAV1 16S rRNA genes were used as standards to construct general 16S rRNA genes and Dehalococcoides 16S rRNA genes calibration curves. The slopes of the calibration curves for general 16S rRNA genes and Dehalococcoides 16S rRNA genes were −3.46 and −3.94, and the Y intercepts were 38 and 44.2, respectively.

### Results and Discussions

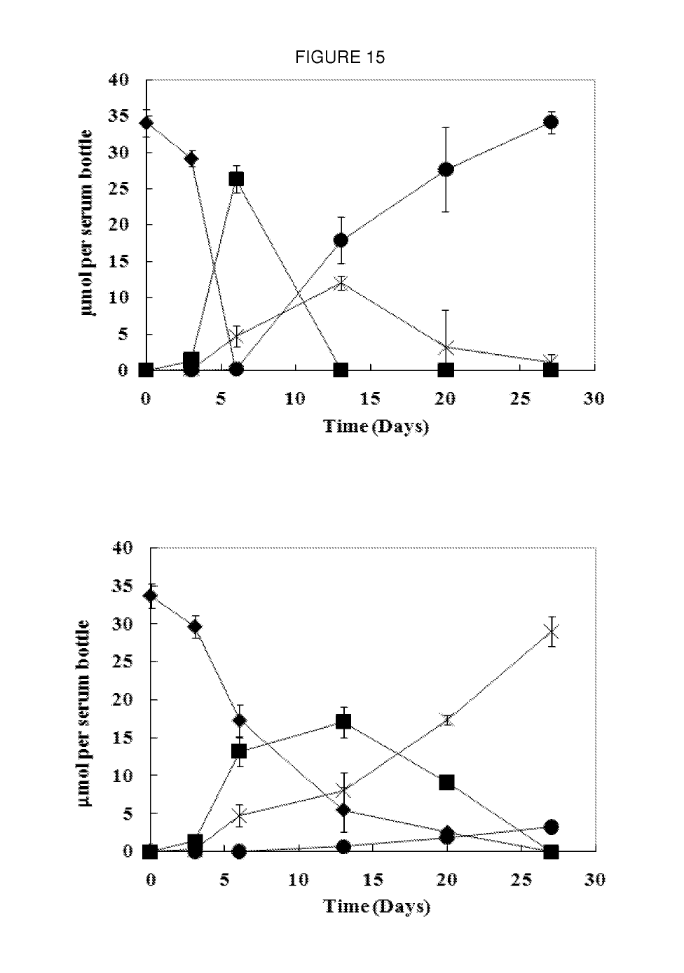

Effect of TCC on TCE Reductive Dechlorination by Bio-Dechlor INOCULUM. The performance of BDI cultures in the presence or absence of TCC is shown in FIG. 15. TCE reductive dechlorination patterns and rates in BDI cultures with 0.15 μM (50 ppb) and 0.015 μM TCC (5 ppb) were similar to the BDI culture without TCC added and were not shown. It took about 6 days to dechlorinate 33 μmol TCE into less chlorinated ethenes in BDI cultures without TCC, and the complete TCE dechlorination took 27 days (FIG. 15). However, TCE reductive dechlorination was much slower in BDI cultures with 1.5 μM TCC (500 ppb), than in other cultures. In this case, by the 27^{th} day, 90% of TCE was transformed to VC and 10% of TCE was completely dechlorinated to ethene (FIG. 15). cis-DCE production and subsequent dechlorination in BDI with 1.5 μM TCC was also much slower than that in other cultures. The build-up of VC was significant. VC and ethene transformation in BDI cultures with 1.5 μM TCC was also significantly slower than that in the other cultures; however, reductive dechlorination did not stop at VC and thus ethene was slowly produced. If the monitoring time was lengthened, complete reductive dechlorination process to ethene could have been observed.

The maximum utilization rate and the maximum formation rate of chloroethenes and ethene in BDI cultures with or without TCC are listed in Table 9. 1.5 μM TCC slowed down the whole dechlorination process, except the VC formation process. The maximum utilization rate of cis-DCE and VC and the maximum formation rate of ethene are obviously less than that in BDI cultures without TCC. Therefore, 1.5 μM TCC significantly inhibited the dechlorination process from VC to ethene in terms of comparing the maximum dechlorination rates.

TABLE 9
Dechlorination rates in BDI cultures with or without TCC. (Rates were
calculated based on the average concentration of triplicate samples.)

| | Maximum | | | Maximum | | |
|---|---|---|---|---|---|---|
| | utilization rate | | | formation rate | | |
| | (μmol vial⁻¹ day⁻¹) | | | (μmol vial⁻¹ day⁻¹) | | |
| | | cis- | | cis- | | |
| Cultures | TCE | DCE | VC | DCE | VC | Ethene |
| BDI | 9.69 | 3.76 | 1.27 | 8.32 | 1.53 | 2.54 |
| BDI + 1.5 μM TCC | 4.22 | 1.29 | NA | 3.94 | 1.65 | 0.19 |
| BDI + 0.15 μM TCC | 7.47 | 2.6 | 0.33 | 7.04 | 1.38 | 3.3 |
| BDI + 0.015 μM TCC | 9.23 | 2.99 | 1.35 | 7.98 | 1.11 | 2.32 |

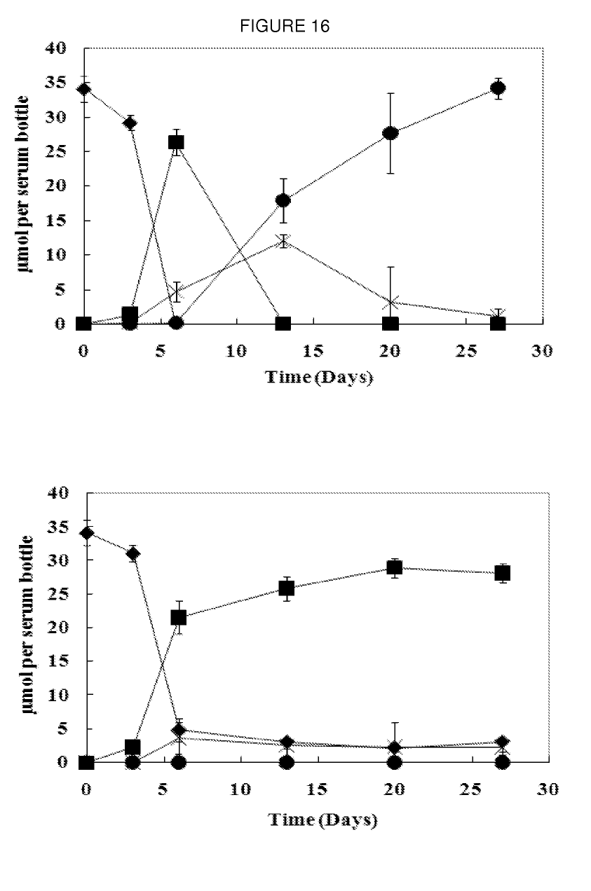

Effect of TCS on TCE Reductive Dechlorination by Bio-Dechlor INOCULUM. The performance of BDI cultures in the presence or absence of TCS is shown in FIG. 16. Complete reductive dechlorination of 35 μmol TCE by BDI cultures without TCS took 27 days. All TCE was transformed into less-chlorinated ethenes in 6 days and ethene production started on the 6^{th} day. In the BDI culture exposed to 35 μM TCS the main accumulated byproduct was cis-DCE, and 84% of TCE was transformed to cis-DCE, with 7% of TCE to VC within 27 days. No ethene was produced by this culture (FIG. 16). The performances of BDI cultures containing 3.5 μM and 0.35 μM TCS are similar to the BDI culture without TCS, and these data are not shown.

In the BDI culture with 35 μM TCS approximately 9% TCE was not dechlorinated and persistent in the culture for the 27-day experiment. cis-DCE was produced more slowly in BDI with 35 μM TCS than in the other cultures, and cis-DCE was not dechlorinated but remained in the culture. Even at the end of the experiment, cis-DCE was still the dominant byproduct of TCE reductive dechlorination in BDI amended with 35 μM TCS. In BDI without TCS all cis-DCE was transformed into following less-chlorinated byproducts in 13 days, which was faster than in other cultures. The maximum cis-DCE utilization rates in BDI without TCS, with 3.5 μM TCS and 0.35 μM TCS were 3.76, 2.39, and 2.72 μmol vial⁻¹ day⁻¹, respectively, so 3.5 μM TCS and 0.35 μM TCS did not have significant effects on cis-DCE transformation in BDI cultures.

Table 10 lists the maximum utilization rate and the maximum formation rate of chloroethenes and ethene in BDI cultures with or without TCS. In BDI cultures with 35 μM TCS, the maximum utilization rate of cis-DCE and VC and the maximum formation rate of ethene were obviously less than that in BDI cultures without TCS. Therefore, 35 μM TCS significantly inhibited the cis-DCE and VC dechlorination process and the ethene production process. But the dechlorination from TCE to cis-DCE and the formation process of VC were not affected by 35 μM TCS. Thus, the dechlorination from VC to ethene was most sensitive to the high concentration of TCS in terms of the maximum dechlorination rates.

TABLE 10
Dechlorination rates in BDI cultures with or without TCS. (Rates were
calculated based on the average concentration of triplicate samples.)

| | Maximum | | | Maximum | | |
|---|---|---|---|---|---|---|
| | utilization rate | | | formation rate | | |
| | (μmol vial⁻¹ day⁻¹) | | | (μmol vial⁻¹ day⁻¹) | | |
| | | cis- | | cis- | | |
| Cultures | TCE | DCE | VC | DCE | VC | Ethene |
| BDI | 9.69 | 3.76 | 1.27 | 8.32 | 1.53 | 2.54 |
| BDI + 35 μM TCS | 8.75 | 0.11 | NA | 6.41 | 1.21 | NA |
| BDI + 3.5 μM TCS | 9.36 | 2.4 | 0.62 | 7.24 | 1.54 | 3.95 |
| BDI + 0.35 μM TCS | 9.58 | 2.72 | 1.14 | 6.69 | 1.14 | 2.61 |

There was no significant difference in VC and ethene transformation among BDI cultures without TCS, with 3.5 μM and 0.35 μM TCS. In BDI cultures amended with 35 μM TCS, only 3.6 μmol VC was produced and then maintained in the culture. In BDI cultures without TCS, with 3.5 μM and 0.35 μM TCS, all chlorinated ethenes were reduced to ethene in 27 days. But no ethene was produced in BDI with 35 μM TCS, so 35 μM TCS showed a significant inhibition on TCE reductive dechlorination by BDI culture, and this inhibition is stronger than 1.5 μM TCC, since there was 10% of TCE transformed into ethene by BDI culture amended with 1.5 μM TCC.

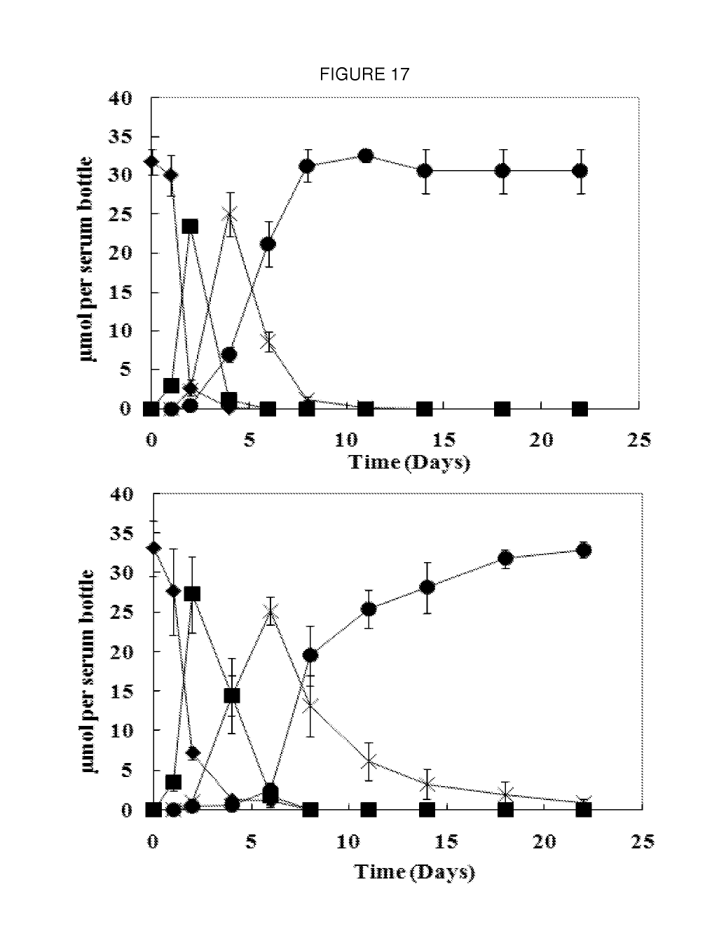

Effect of TCC or TCS on TCE Reductive Dechlorination by DehaloR̂2 Culture. Reductive dechlorination of TCE in DehaloR̂2 culture without TCS and TCC is shown in FIGS. 17 to 20. Complete removal of 32 μmol TCE in 100 ml DehaloR² culture was achieved in less than 11 days, and 74% of TCE was transformed into cis-DCE in 2 days. On the 4^{th} day, VC became the dominant byproduct, and on the 8^{th} day, 96% of TCE was completely dechlorinated to ethene.

Complete TCE reductive dechlorination in DehaloR̂2 culture amended with 0.15 μM TCC took approximately 22 days (FIG. 17), almost two times longer than the DehaloR̂2 culture without TCS or TCC. In the DehaloR̂2 culture with 0.15 μM TCC added, cis-DCE became the main dechlorination product by the 2^{nd} day, and VC was the dominant byproduct of TCE reduction after 6 days. On the 2^{nd} day, ethene production started. Reductive dechlorination of 80% of 33 μmol TCE into ethene took 11 days.

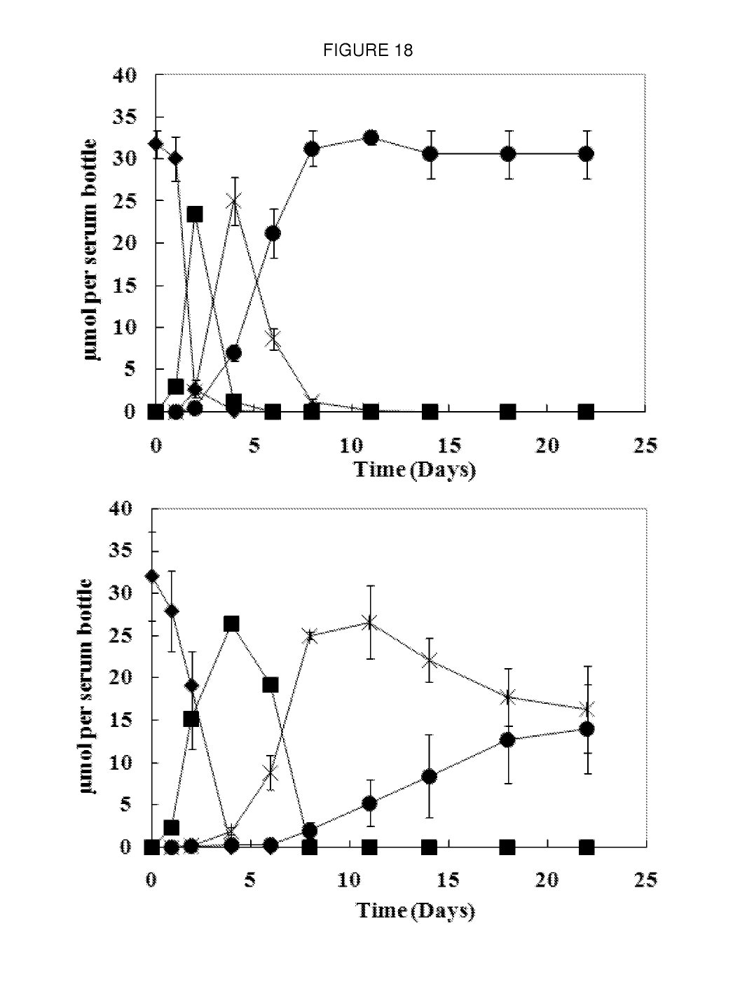

TCE Reductive dechlorination in DehaloR̂2 culture with 1.5 μM TCC is shown in FIG. 18. In this case, the reductive dechlorination process of TCE to cis-DCE took about 4 days. Ethene production started at day six. At the 8^{th} day, VC was the main byproduct. After the 18^{th} day, the ethene production rate and the VC dechlorination rate slowed down. About 46% of 32 μmol TCE was completely dechlorinated into ethene in the 22-day period.

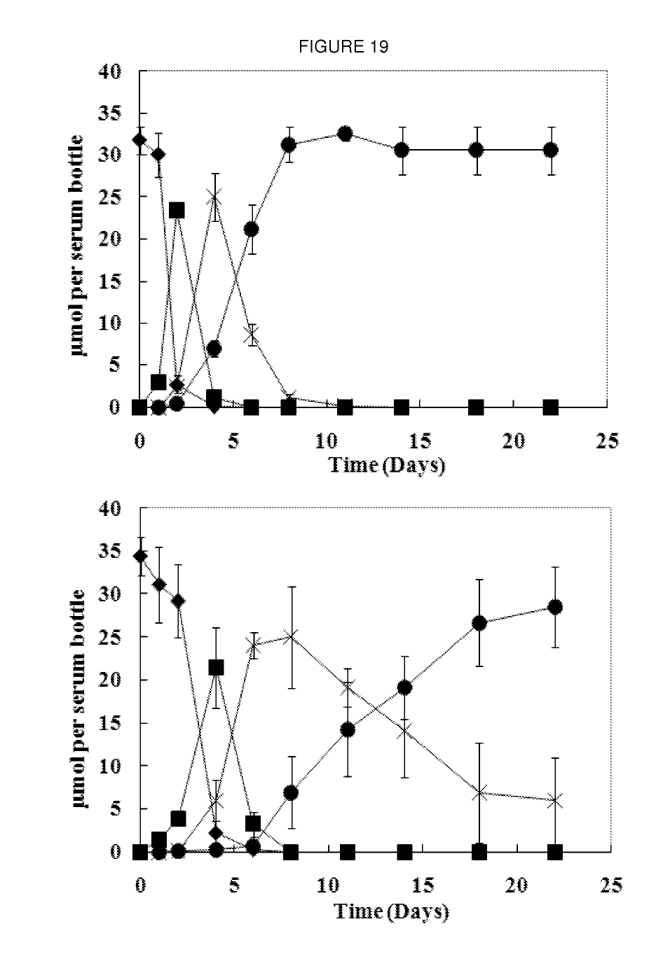

The DehaloR̂2 culture with 3.5 μM TCS exhibited an average TCE reductive dechlorination rate that was slower than that with DehaloR̂2 culture amended with the 0.15 μM TCC (FIG. 19). The error bars were much bigger than that in other figures because the performance of the triplicate cultures with the same TCS concentration differed. TCE reductive dechlorination to cis-DCE took about 4 days. On the 6^{th} day, VC became the dominant byproduct, and at the 18^{th} day, 79% of 34 μmol TCE was completely dechlorinated into ethene. Reductive dechlorination slowed down after this point.

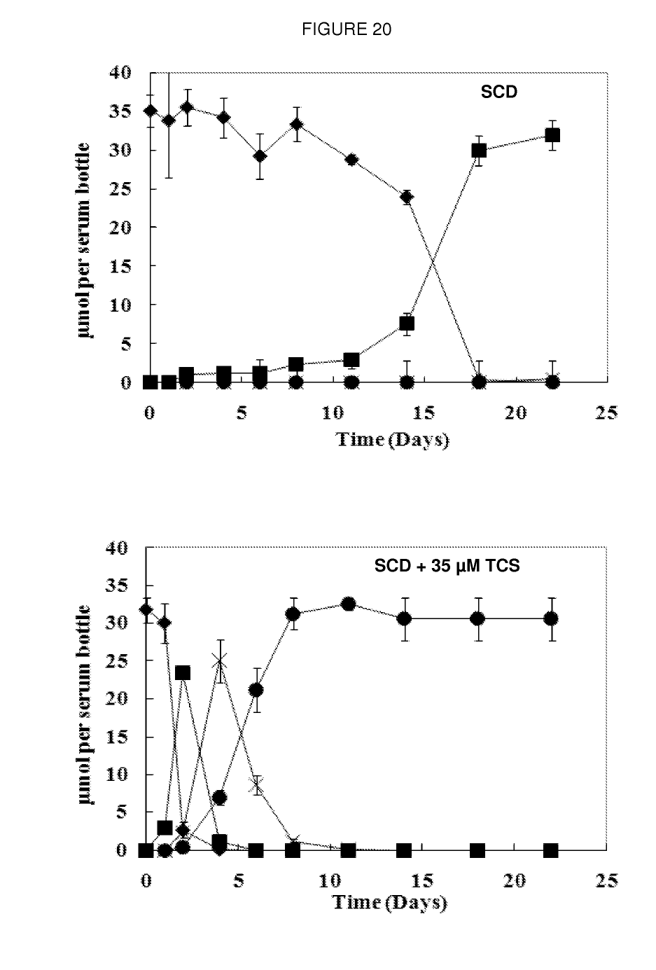

TCE reductive dechlorination in DehaloR̂2 culture amended with 35 μM TCS exhibited the slowest reduction rate of TCE (FIG. 20). cis-DCE became the dominant byproduct at the 18^{th} day, and only 1% of TCE was transformed into VC within 22 days. Additionally, no ethene was produced in the 22-day experiment.

According to the dechlorination rates shown in Table 11, 0.15 μM TCC only slowed down the whole process, but the TCE dechlorination followed the same pattern. 3.5 μM TCS and 1.5 μM TCC significant inhibited the transformation process from VC to ethene. In DehaloR̂2 cultures with 35 μM TCS, the maximum utilization rate of TCE and the maximum formation rate of cis-DCE and VC were much less than that in other cultures and there was no transformation from VC to ethene. Thus, 35 μM TCS exerted the most significant effects on TCE reductive dechlorination by comparing the maximum dechlorination rates.

TABLE 11
Dechlorination rates in DehaloR\hat{ }2 cultures with or
without TCC or TCS. (Rates are based on the
average concentration of triplicate samples.)

| | Maximum | | | Maximum | | |
|---|---|---|---|---|---|---|
| | utilization rate | | | formation rate | | |
| | (μmol vial⁻¹ day⁻¹) | | | (μmol vial⁻¹ day⁻¹) | | |
| | | cis- | | cis- | | |
| Cultures | TCE | DCE | VC | DCE | VC | Ethene |
| SCD | 27.37 | 11.07 | 8.22 | 20.34 | 11.33 | 7.09 |
| SCD + 0.15 μM TCC | 20.45 | 6.40 | 6.01 | 23.87 | 6.72 | 8.54 |
| SCD + 1.5 μM TCC | 9.54 | 9.55 | 1.48 | 12.83 | 8.12 | 1.08 |
| SCD + 3.5 μM TCS | 13.47 | 9.10 | 1.96 | 8.79 | 8.99 | 3.11 |
| SCD + 35 μM TCS | 5.90 | NA | NA | 5.59 | 0.11 | NA |

TCC and TCS had strong effects on TCE reductive dechlorination process in DehaloR̂2 cultures. These results provided supporting evidence for the hypothesis in Example 1 that the incomplete TCE reductive dechlorination in sediment microcosms may have been caused by high concentrations of TCC and TCS in the sediment samples. The inhibition by 35 μM TCS was more obvious than that of 1.5 μM TCC, as 35 μM TCS caused reductive dechlorination of TCE to stop at VC. The stronger impact of TCS was related at least in part to its much higher concentration, since lower concentration of TCS gave results more similar to TCS.

TCC at 0.15 μM and TCS at 3.5 μM showed more significant effects on DehaloR̂2 cultures than they did on BDI. Perhaps the microbial community of DehaloR̂2 culture is more sensitive to TCC or TCS than that of BDI culture.

The mechanisms of the inhibition by TCC and TCS on TCE reductive dechlorination are not clear, but possible reasons may include: 1) TCC or TCS may cross the bacteria cell membrane and block the TCE reductive dechlorination process, especially at 1.5 μM TCC and 35 μM TCS respectively, which are much higher than the solubility of TCC and TCS in water at room temperature; 2) TCC or TCS can penetrate the cell membrane of TCE dechlorinating bacteria in the mixed cultures and inhibit the activity of reductases, such as enoyl-acyl carrier-protein reductase (ENR); 3) TCC or TCS may be reductively dechlorinated by Dehalococcoides or other bacteria in mixed culture, creating an electron-donor competition between TCC/TCS and TCE, so TCE dechlorinators do not have enough electron donors to support the reductive dechlorination process; 4) TCC or TCS may inhibit other bacteria in the microbial community, such as fermentors, which are responsible for providing carbon source and electron donor to TCE dechlorinators, thus the reductive dechlorination process is suppressed.

The first and second reasons are hard to prove in this study, since both the BDI cultures and sediment-D cultures are mixed cultures. Lack of electron donor can be one possible reason. DehaloR̂2 culture was generated from sediment core D microcosm. The reductive dechlorination of TCC was found at the location where this sediment sample was obtained from, and the TCC dechlorinating byproducts, such as dichlorocarbanilide (DCC), monochlorocarbanilide (MCC) and nonchlorinated carbanilide (NCC) were detected in the sediment sample (Miller et al., 2008). This is significant in that the microorganisms in sediment core D may have the potential to reductively dechlorinate TCC or TCS. If the microorganisms capable of degrading TCC or TCS exist in the sediment and they were enriched in the sediment-free cultures, then they could compete with chlorothene reductive dechlorinating bacteria, such as Dehalococcoides, for electron donors. Another possibility is that Dehalococcoides or other bacteria may also reductively dechlorinate TCC or TCS, and there are not enough electron donors to support all these reductive dechlorination process, so the TCE dechlorination process was inhibited. In order to verify these assumptions, the TCC and TCS concentrations in BDI cultures and DehaloR̂2 cultures need to be measured to investigate if there are any TCC or TCS reductive dechlorination byproducts.

However, on the other hand, the third possible inhibition mechanism may be not very convincing, because the dechlorinations of 35 μM TCS and 1.5 μM TCC need 105 μM and 4.5 μM of electron donors (H₂), respectively, which are less than the electron donors needed by 33 μmol TCE (263 μM). The electron-donor competition between TCC/TCS and TCE is not likely to excerpt strong effects on TCE dechlorination in this case.

The consumption of electron donor by other bacteria that are less sensitive to TCC or TCS instead of TCE dechlorinators can also lead to the lack of electron donors, and the hydrogen producers such as part of fermentors in the culture may be stressed by TCC or TCS, and thus less hydrogen were produced for Dehalococcoides. Therefore, TCE dechlorinators do not have sufficient electron donors and TCE dechlorination process is inhibited.

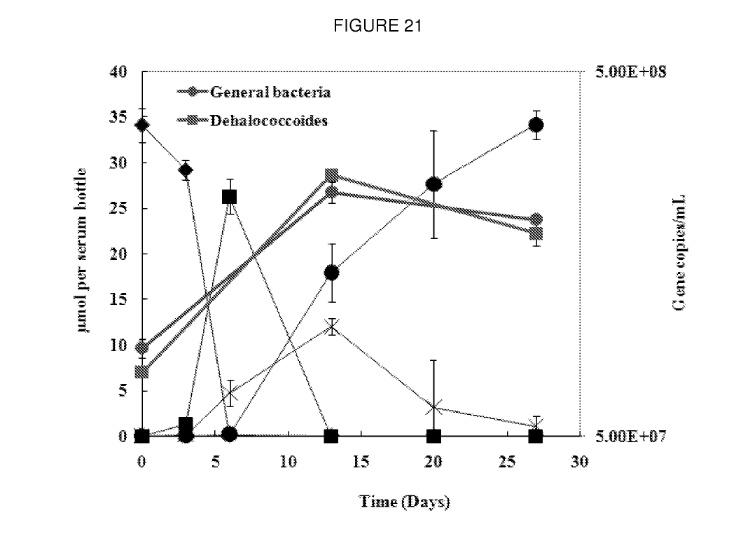

Quantification of General Bacteria and Dehalococcoides. FIG. 21 shows the quantitative real-time PCR (qRT-PCR) results of general bacteria 16S rRNA gene copy numbers and Dehalococcoides 16S rRNA gene copy numbers in the BDI cultures without TCC & TCS as a function of TCE dechlorination. In BDI cultures without TCC & TCS, general 16S rRNA gene copies increased from 8.7×10⁷ to 2.3×10⁸ gene copies/ml in 13 days and then slowly decreased to 2.0×10⁶ on day 27. Dehalococcoides 16S rRNA gene copy number has a similar trend: increased from 7.5×10⁶ to 2.6×10⁸ gene copies/ml in 13 days, and then slowly decreased to 1.8×10⁸ gene copies/ml later. This result is reasonable because 60% of TCE was already dechlorinated into ethene at day 13 and reductive dechlorination was accomplished before day 27. Dehalococcoides may have been starving at the last sampling date and the cells started to lyse, so the Dehalococcoides 16S rRNA gene copies decreased in the last 14 days.

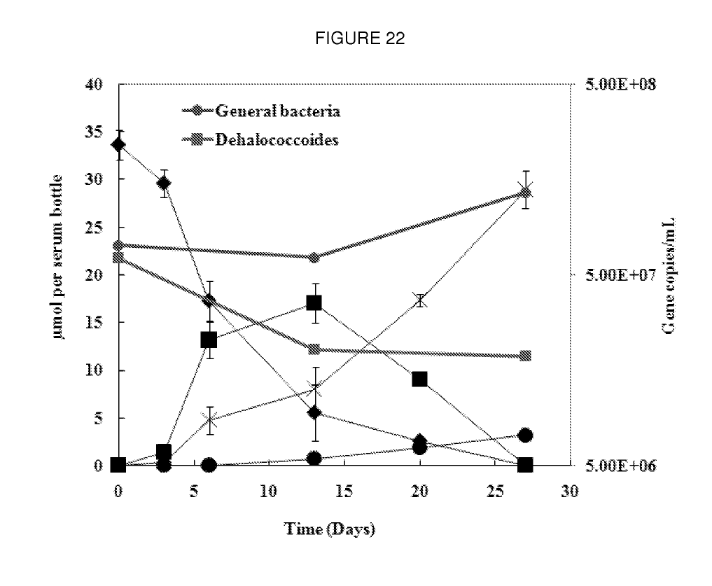

General bacteria 16S rRNA gene copy numbers and Dehalococcoides 16S rRNA gene copy numbers in the BDI cultures with 1.5 μM TCC as a function of TCE dechlorination reactions are shown in FIG. 22. General bacteria 16S rRNA gene copies increased from 7.2×10⁷ to 1.4×10⁸ gene copies/ml in 27 days. But Dehalococcoides 16S rRNA gene copies decreased from 6.1×10⁷ to 1.9×10⁷ gene copies/ml, indicating that 1.5 μM TCC significantly inhibited the growth of Dehalococcoides and this explains why TCE was not completely dechlorinated to ethene in this culture.

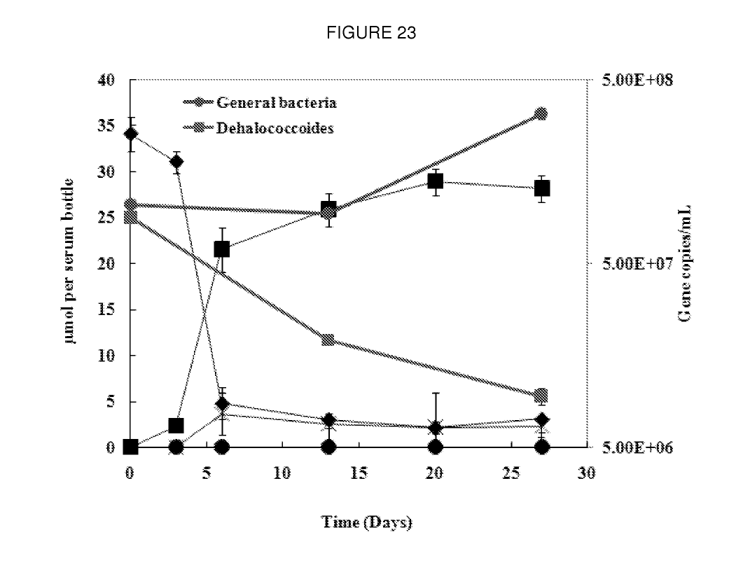

FIG. 23 shows the qRT-PCR results of general bacteria 16S rRNA gene copies and Dehalococcoides 16S rRNA gene copies in the BDI cultures with 35 μM TCS as a function of TCE dechlorination reactions. The general bacteria 16S rRNA gene copies increased from 1.0×10⁸ to 3.3×10⁸ gene copies/ml in the experiment period. However, Dehalococcoides 16S rRNA gene copies decreased from 8.9×10⁷ to 9.4×10⁶ gene copies/ml in 27 days. Thus, 35 μM TCS significantly inhibited the growth of Dehalococcoides, and this growth inhibition effect was more obvious than the inhibition effect of 1.5 μM TCC. TCE reductive dechlorination stopped at cis-DCE in BDI with 35 μM TCS, but the TCE dechlorination in BDI with 1.5 μM TCC went further to VC and ethene. Thus, the qRT-PCR results were consistant with the TCE reductive dechlorination results in our study.

On the other hand, general bacteria 16S rRNA gene copy numbers increased in BDI with 1.5 μM TCC or 35 μM TCS, which implies that other dechlorinators responsible for partial TCE dechlorination in BDI culture were less affected by TCC and TCS than Dehalococcoides. This explains why TCE dechlorination was not complete in BDI cultures amended with 1.5 μM TCC or 35 μM TCS.

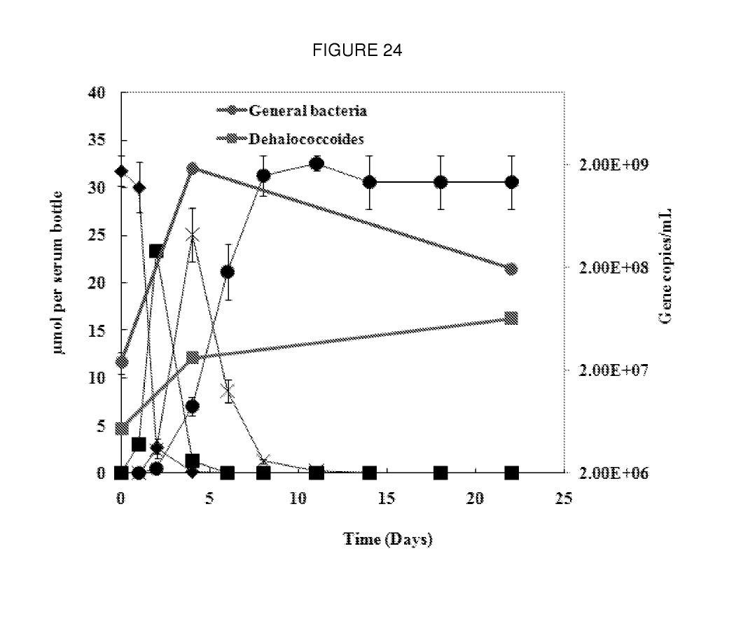

FIG. 24 shows the qRT-PCR results of general bacteria and Dehalococcoides 16S rRNA gene copy numbers in the DehaloR̂2 cultures without TCC & TCS for day 0, 4 and 22 as a function of TCE dechlorination reactions. General bacteria 16S rRNA gene copies increased from 2.4×10⁷ to 1.8×10⁹ gene copies/ml in the initial 4 days, and then decreased to 1.9×10⁹ gene copies/ml. Because the electron donors and acceptors were ample and reductive dechlorination activity was vigorous at the beginning, the microorganisms grew quickly in the initial 4 days. According to the pyrosequencing data in Example 1, the main group of bacteria in DehaloR̂2 is acetogens. Therefore, after 5 mM lactate was exhausted in the cultures, these bacteria were starving and started to decay.

Dehalococcoides 16S rRNA gene copy number in DehaloR̂2 culture was increasing in the whole monitoring period from 5.5×10⁶ to 6.4×10⁷ gene copies/ml (11.7 times of the initial Dehalococcoides 16S rRNA gene copies). However, if we have tested the Dehalococcoides 16S rRNA gene copies on day 11, the day when complete dechlorination was achieved, the number may be even higher.

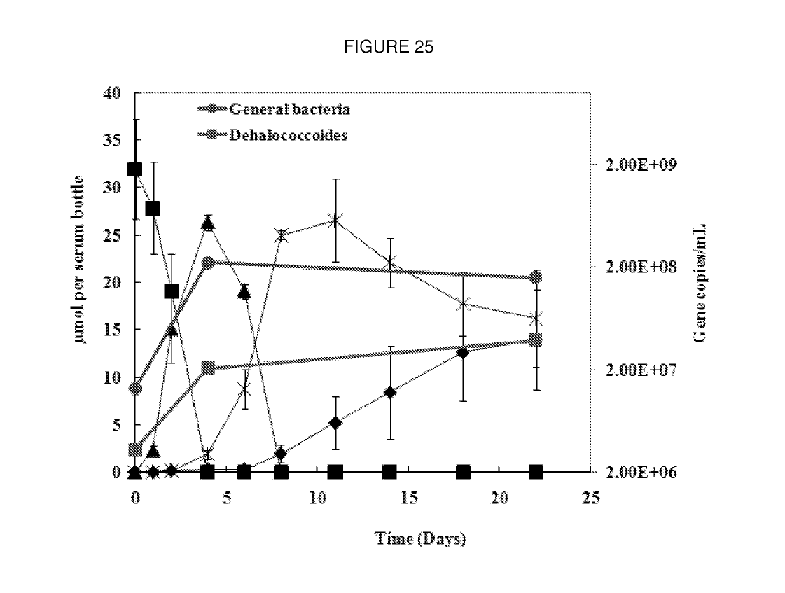

General bacteria 16S rRNA gene copy numbers and Dehalococcoides 16S rRNA gene copy numbers in the DehaloR̂2 cultures with 1.5 μM TCC as a function of TCE dechlorination reactions are shown in FIG. 25. General bacteria 16S rRNA gene copies and Dehalococcoides 16S rRNA gene copies increased 1.3×10⁷ to 1.6×10⁸ gene copies/ml and from 3.3×10⁶ to 3.9×10⁷ gene copies/ml, respectively in 22 days. Interestingly, Dehalococcoides 16S rRNA gene copies in DehaloR̂2 cultures amended with 1.5 μM TCC showed a very similar trend to the DehaloR̂2 cultures without TCC&TCS. The gene copies of Dehalococcoides 16S rRNA in DehaloR̂2 with 1.5 μM TCC on day 22 was 11.6 times of the initial gene copies in this culture. However, the dechlorination data showed that TCE reductive dechlorination in DehaloR̂2 with 1.5 μM TCC was significantly slower than that in DehaloR̂2 without TCC & TCS. Thus, Dehalococcoides in DehaloR̂2 culture may also dechlorinate TCC and get energy from this process to support their growth.

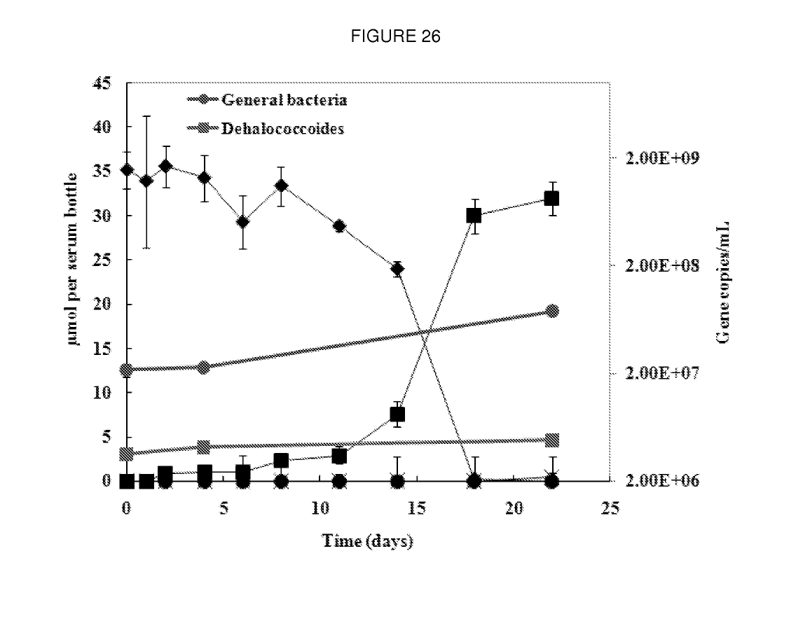

FIG. 26 shows the qRT-PCR results of general bacteria 16S rRNA gene copy numbers and Dehalococcoides 16S rRNA gene copy numbers in the DehaloR̂2 cultures with 35 μM TCS as a function of dechlorination reactions. General bacteria 16S rRNA gene copies increased slowly from 2.2×10⁷ to 7.5×10⁷ gene copies/ml on day 22, which is 3.5 times of the initial number. However, in DehaloR̂2 culture amended with 35 μM TCS Dehalococcoides 16S rRNA gene copy number almost remained constant in the whole experiment period, which means 35 μM TCS hindered the growth of Dehalococcoides in the DehaloR̂2 culture. Nevertheless, according to the TCE dechlorination data, all the TCE in this culture was dechlorinated into cis-DCE, thus other dechlorinators in DehaloR̂2 cultures were responsible for the reductive dechlorination from TCE to cis-DCE and the general bacteria 16S rRNA gene copies increased.

This phenomenon was different in BDI cultures since the Dehalococcoides 16S rRNA gene copy number in BDI cultures amended with 1.5 μM TCC or 35 μM TCS decreased significantly. This is probably because DehaloR̂2 cultures were generated from TCC and TCS containing sediments, and the Dehalococcoides in this culture have somehow acclimated to TCC and TCS exposure.

### CONCLUSION

TCC and TCS inhibition test on BDI cultures and DehaloR̂2 cultures showed that 35 μM TCS and 1.5 μM TCC significantly inhibited TCE reductive dechlorination. 3.5 μM TCS, 0.35 μM TCS, 0.15 μM TCC and 0.015 μM TCC did not show any obvious effect on TCE reductive dechlorination with the BDI culture. However, in DehaloR̂2 cultures 3.5 μM TCS and 0.15 μM TCC slowed down the TCE reductive dechlorination process. Moreover, 35 μM TCS showed a more significant inhibition on TCE reductive dechlorination than 1.5 μM TCC, since there was no ethene produced in the cultures amended with 35 μM TCS.

General bacteria 16S rRNA gene copies and Dehalococcoides 16S rRNA gene copies in BDI cultures or DehaloR̂2 cultures were quantified by qRT-PCR in this study. The qRT-PCR results indicate that 1.5 μM TCC and 35 μM TCS significantly inhibited the growth of Dehalococcoides in BDI cultures, and the inhibitory effect of 35 μM TCS was much stronger than that of 1.5 μM TCC. However, in DehaloR̂2 cultures, 1.5 μM TCC did not exert any inhibitory effect on the growth of Dehalococcoides impling that the Dehalococcoides in DehaloR̂2 cultures may be able reductively dechlorinate TCC. Little growth of Dehalococcoides was observed in DehaloR̂2 cultures added with 35 μM TCS in the whole monitoring period, thus 35 μM TCS significantly inhibited the growth of Dehalococcoides in DehaloR̂2 cultures.

In order to understand the TCC and TCS inhibition on TCE reductive dechlorination deeply, TCC and TCS concentrations in BDI cultures and DehaloR̂2 cultures need to be analyzed, and additional TCC and TCS inhibition tests, including those on Dehalococcoides pure cultures, are recommended. These studies could explore if TCC or TCS inhibition on different Dehalococcoides strains are distinct, and whether the inhibition effects are observable only with Dehalococcoides or also with other bacteria in a mixed culture. This is important fundamental information for the application of in situ TCE bioremediation. For example, the bioremediation of removal TCE from TCC or TCS accumulated sediment requires us to know what concentrations TCC or TCS will or will not inhibit TCE biodegradation, and which culture is better for TCE bioremediation when TCC or TCS are present in the field.

### Example 3

### Summary and Recommendations

Summary: In this study, four sediment microcosms were set up from the estuarial sediments obtained from Chesapeake Bay (CB), Md. I tested TCE reductive dechlorination in these sediment microcosms. At the beginning, there was an 8 to 18 days' lag time, and then TCE was quickly dechlorinated into trans-DCE and cis-DCE, but the reductive dechlorination process stopped at this point. These sediment microcosms produced more trans-DCE than cis-DCE, and the trans-DCE/cis-DCE ratios in the four sediment microcosms ranged from 1.35±0.15 to 1.67±0.15.

In order to exclude the possibility of the competition from methanogenic activity for electron donor in the sediment microcosms, I added 25 mM BES into two sediment microcosms to inhibit methanogens, and I also added 70 μmol TCE and 5 mM lactate into all four sediment microcosms to provide enough electron donor, electron acceptor and carbon source. TCE dechlorination in sediment microcosms still stopped at trans-DCE and cis-DCE after adding 25 mM BES, and the trans-DCE/cis-DCE ratios in TCE reductive dechlorination decreased, which indicated that 25 mM BES inhibited the reductive dechlorination from TCE to trans-DCE. In the two microcosms for which I only added 70 μmol TCE and 5 mM lactate (without BES), the trans-DCE/cis-DCE ratios increased, which implied that adding 70 μmol TCE may be helpful to enrich reductively dechlorinating microorganisms and promote the dechlorination of TCE into cis-DCE.

Complete dechlorination from TCE to ethene in sediment microcosms was not observed in a period of 7 months. After this, 12 sediment-free cultures were generated. In the sediment-free cultures TCE was rapidly dechlorinated to ethene, especially in the DehaloR² cultures where TCE complete dechlorination to ethene was achieved in 10 days. It took about 32 days, 25 days, and 54 days, respectively, for complete TCE reductive dechlorination in SCAB cultures, SCAT cultures, and SCDBT cultures. According to the maximum utilization rate, maximum formation rate of chlorothenes, and the time for complete TCE dechlorination, the sediment-free cultures presented different TCE dechlorination rates: DehaloR̂2 culture>SCAT culture>SCAB culture>SCDBT culture. The TCE reductive dechlorination results in sediment-free cultures also proved that TCC, TCS and BES inhibited TCE dechlorination by slowing down TCE dechlorination rates. Furthermore, Generation II DehaloR̂2 cultures were developed and these completely dechlorinated 40 μmol TCE and 20 μmol PCE in 7 and 9 days, respectively.

Dehalococcoides was not detected in the sediment A and D samples by PCR, nested PCR and pyrosequencing. In the DehaloR̂2 and SCAT, we amplified DNA with PCR primers targeting Dehalococcoides. Based on pyrosequencing data, Dehalococcoides sequences were 0.4% and 0.2% of the analyzed microbial community sequences in DehaloR̂2 and SCAT respectively. Proteobacteria phylum is dominant in sediment microbial community, and in the sediment-free cultures the dominant bacteria shifted to Firmicutes phylum. In DehaloR̂2 and SCAT, especially in DehaloR̂2 culture, although Dehalococcoides was a small group of bacteria, other putative dechlorinating bacteria, such as Acetobacterium, Geobacter, Trichlorobacter and Clostridium sequences, increased and were present in the DNA analysed. These microorganisms could interact with Dehalococcoides to achieve complete reductive dechlorination of PCE or TCE to ethene.

The DehaloR² culture is a valuable bacterial source for TCE or PCE bioremediation, the potential of this culture is great because the dechlorination of TCE and PCE can proceed much faster than with other cultures, such as BDI. The BDI culture takes about 35 days to dechlorinate 22.5 μmol TCE per serum bottle into ethene, while the DehaloR̂2 culture only takes about 5 days to dechlorinate 40 μmol TCE under the same conditions. It is also valuable for research since 3 complete experiments can be done with DehaloR̂2 in the same time it would take to run one experiment with BDI.

The experiments performed for testing the effect of TCC and TCS on BDI cultures and DehaloR̂2 cultures showed that 35 μM TCS and 1.5 μM TCC significantly inhibited TCE reductive dechlorination. No ethene was produced in the cultures added with 35 μM TCS, and in the cultures amended with 1.5 μM TCC, TCE dechlorination was not complete. There appeared to be no obvious effect on TCE reductive dechlorination with the BDI culture when I added 3.5 μM TCS, 0.35 μM TCS, 0.15 μM TCC and 0.015 μM TCC. However, when 3.5 μM TCS and 0.15 μM TCC was added to DehaloR̂2 cultures TCE reductive dechlorination rates were slower than the dechlorination rates of DehaloR̂2 cultures without TCC and TCS exposure. Overall, 35 μM TCS showed a more significant inhibition on TCE reductive dechlorination than 1.5 μM TCC in both cultures tested.

qRT-PCR was used for the quantification of general bacteria 16S rRNA gene copies and Dehalococcoides 16S rRNA gene copies in BDI cultures or DehaloR̂2 cultures in this study. Dehalococcoides 16S rRNA gene copies dramatically decreased in BDI cultures added with 1.5 μM TCC or 35 μM TCS, and the inhibitory effect of 35 μM TCS was much stronger than that of 1.5 μM TCC. However, 1.5 μM TCC did not exert any inhibitory effects on Dehalococcoides 16S rRNA gene copies in DehaloR̂2 cultures, implying that the Dehalococcoides in DehaloR̂2 cultures may be able to reductively dechlorinate TCC. In DehaloR̂2 cultures amended with 35 μM TCS, Dehalococcoides 16S rRNA gene copies almost maintained at the same level in the whole monitoring period, thus 35 μM TCS significantly inhibited the growth of Dehalococcoides in DehaloR̂2 cultures which is consistant with the TCE dechlorination data.

Based on the results and major challenges arising from this study, the following research is suggested to better understand TCE reductive dechlorination process and some associated factors:

(1) Explore the effect of BES on reductive dechlorination from TCE to trans-DCE;

(2) Investigate the possible reductive dechlorination of TCC and TCS by the sediment-free culture;

(3) Identify and isolate microbial species capable of reductive dechlorination to seek new highly efficient dechlorinating bacteria like Dehalococcoides.

To study the TCC and TCS inhibition on TCE reductive dechlorination, it is necessary to monitor TCC, TCS and their possible dechlorination byproducts and their respective concentrations in BDI cultures and DehaloR̂2 cultures. Additionally, TCC and TCS inhibition tests on Dehalococcoides pure cultures are necessary, because it is unknown whether TCC and TCS inhibition on different strains of Dehalococcoides is diverse, and what are the inhibition effects on other bacteria besides Dehalococcoides in the mixed culture.

### Example 4

### Additional Materials and Methods

Recipe For Anaerobic Media

The anaerobic media was prepared using the Hungate technique and based on the Löffler et al. recipe (Löffler et al., 2005).

The following steps describe the procedure:

1. For preparing 1 liter medium, following solutions are added into water: 10 ml of 100-fold concentrated salts stock solution; 1 ml trace element solution A; 1 ml trace element solution B; 0.25 ml 0.1% (w/v) resazurin stock solution; 500 mM sodium lactate solution, volume is based on the electron acceptor (i.e. TCE) concentration, or add 0.2 μm-filter-sterilized lactate stock solution after autoclaving; and add D.I. water to a total volume of 1 liter.

2. Transfer the medium to a 2 L round flask (Chemglass) and heat it and boil for 10 min. Use a condenser and a cooler system to avoid evaporative water loss.

3. Then transfer flask to an ice bath and cool the medium down to room temperature under a stream of N2 gas.

4. Add 30 mM NaHCO₃, 0.2 Mm L-cysteine, and 0.2 mM Na2S. Adjust the pH to 7-7.5 by purging the mixed gas which contains 20% CO₂ and 80% N2.

5. Dispense medium into serum bottles which have been purged with N2. The volume of medium should be less than 75% of the total capacity of serum bottle.

6. Cap the serum bottle with thick black butyl rubber stoppers and aluminum crimps.

7. Autoclave the serum bottles with medium and keep them in a bottom-up position.

8. After autoclave, keep the serum bottles in dark at room temperature or 4° C.

9. Before using the medium, add 1 ml ATCC mixed vitamin solution (Catalog No. MD-VS) and 0.25 ml 20 mg/L vitamin B12 solution into 98 ml medium, and the vitamin solution are sterilized by 0.2 μm sterile filter.

Procedure For Setting Up Microcosms

Microcosms were set up in an anaerobic chamber with H2 concentration is 3% (Coy laboratory products Inc. Grass Lake, Mich.). The following procedure is used to set up sediment microcosms:

1. Prepare anaerobic medium and add mixed vitamin solution, vitamin B12 solution and lactate solution.

2. Autoclave empty serum bottle, Ellipso-Spoon samplers, and thick black butyl rubber stoppers.

3. Transfer sediment samples, anaerobic medium, empty serum bottles, Ellipso-Spoon samplers, butyl rubber stoppers, crimper, decrimper, TCE, TCC stock solution, TCS stock solution, 10 μL syringe, pipettes and tips into anaerobic chamber.

4. Wait for half hour until the oxygen concentration is zero.

5. Weigh empty serum bottle and set it as zero, then weigh 10 g sediment A or D in serum bottles.

6. Add 90 ml anaerobic medium into serum bottles.

7. Add 100 μL TCC stock solution and 100 μL TCS stock solution according to the design of the experiment.

8. Cap the serum bottles with thick black butyl rubber stoppers and aluminum crimps.

9. Inject 4.5 μL pure TCE with air-tight syringe (Hamilton Company, Reno, Nev.) into serum bottles.

10. According to Table 1 to 4, generate 4 bottles of sediment microcosms, and keep them in the dark incubator at 30° C.

TABLE 1
Sediment A + TCE + TCC + TCS

| | | | | | 100 mL soln | |
|---|---|---|---|---|---|---|
| | Final | | Stock | | (160 mL serum | |
| Constituent | conc. | | soln | | bottles) | |
| Media Solution | | | | | 87.5 | mL |
| vitamin stock soln | | | 100 x | | 1 | mL |
| (filter sterilized) | | | pre-made | | | |
| cyanocobalamine (B12) | 0.5 | mg/L | 10 | mg/L | 0.5 | mL |
| Lactate | 2 | mM | 200 | mM | 1 | mL |
| TCE | 0.5 | mM | 99.8% | | 4.49 | μL |
| TCC | 20 | μg/L | 20 | mg/L | 100 | μL |
| TCS | 20 | μg/L | 20 | mg/L | 100 | μL |

| | | |
|---|---|---|
| Sediment A | ~10 | g |

TABLE 2
Sediment D + TCE + TCC + TCS

| | | | | | 100 mL soln | |
|---|---|---|---|---|---|---|
| | Final | | Stock | | (160 mL serum | |
| Constituent | conc. | | soln | | bottles) | |
| Media Solution | | | | | 87.5 | mL |
| vitamin stock soln | | | 100 x | | 1 | mL |
| (filter sterilized) | | | pre-made | | | |
| cyanocobalamine (B12) | 0.5 | mg/L | 10 | mg/L | 0.5 | mL |
| Lactate | 2 | mM | 200 | mM | 1 | mL |
| TCE | 0.5 | mM | 99.8% | | 4.49 | μL |
| TCC | 20 | μg/L | 20 | mg/L | 100 | μL |
| TCS | 20 | μg/L | 20 | mg/L | 100 | μL |

| | | |
|---|---|---|
| Sediment D | ~10 | g |

TABLE 3
Sediment A + TCE

| | | | | | 100 mL soln | |
|---|---|---|---|---|---|---|
| | Final | | Stock | | (160 mL serum | |
| Constituent | conc. | | soln | | bottles) | |
| Media Solution | | | | | 87.5 | mL |
| vitamin stock soln | | | 100 x | | 1 | mL |
| (filter sterilized) | | | pre-made | | | |
| cyanocobalamine (B12) | 0.5 | mg/L | 10 | mg/L | 0.5 | mL |
| Lactate | 2 | mM | 200 | mM | 1 | mL |

| | | | | | |
|---|---|---|---|---|---|
| TCE | 0.5 | mM | 99.8% | 4.49 | μL |

| | | |
|---|---|---|
| Sediment A | ~10 | g |

TABLE 4
Sediment D + TCE

| | | | | | 100 mL soln | |
|---|---|---|---|---|---|---|
| | Final | | Stock | | (160 mL serum | |
| Constituent | conc. | | soln | | bottles) | |
| Media Solution | | | | | 87.5 | mL |
| vitamin stock soln | | | 100 x | | 1 | mL |
| (filter sterilized) | | | pre-made | | | |
| cyanocobalamine (B12) | 0.5 | mg/L | 10 | mg/L | 0.5 | mL |
| Lactate | 2 | mM | 200 | mM | 1 | Ml |

| | | | | | |
|---|---|---|---|---|---|
| TCE | 0.5 | mM | 99.8% | 4.49 | μL |

| | | |
|---|---|---|
| Sediment D | ~10 | g |

DNA Extraction Protocol For Sediment

(FASTID Kit Combined with Mio Bio Bead Tube and SDS)

1. For each extraction, take 1,000 μl of Genomic Lyse buffer and premix with 10 μl of Proteinase K solution.

Note: If precipitates occur in the Genomic Lyse buffer due to cold temperature the buffer must be warmed to 20 to 30° C. and mixed up in order to completely solubilize its contents.

2. In a labeled 2 ml vial add 400 mg of ground and homogenized sample and mix with 1,000 μl Genomic Lyse buffer premixed with Proteinase K (#1).

3. Vortex thoroughly until a homogeneous slurry is obtained.

4. Incubate at 65° C. for 30 minutes.

5. Transfer the lysate into a Mo Bio Bead Tube (Mo Bio Laboratories, Carlsbad, Calif.), add 0.5 μL 10% SDS solution, and shaken horizontally on a Vortex mixer at maximum speed for 10 min.

6. Spin at about 10,000 rpm for 5 minutes in a microcentrifuge.

7. Take 500 μl of supernatant and transfer it into a new labeled 2 ml vial.

Note: If less volume is available, transfer as much as possible without taking sediment. If more volume is available, disperse it into two 2 ml vials and later load them on the same binding column.

8. Add an equal amount of Genomic Bind buffer and vortex briefly. If, in very rare cases, the indicator color changes, refer to troubleshooting.

9. Spin at about 10,000 rpm for 5 minutes in a microcentrifuge.

10. Pass the supernatant through the DNA Binding Column. Centrifuge at 3,000 rpm for 5 minute.

Note: Spills of buffers should be cleaned up thoroughly. Residues of salts contained in the buffer solution can cause corrosions.

11. Wash one time with 800 μl of Genomic Wash buffer and spin 30 sec at the maximum speed, and discard the flow through.

12. Wash three times with 800 μl 75% ethanol, and spin 30 sec at the maximum speed and discard the flow through.

13. Spin 1 min at the maximum speed in a microcentrifuge.

14. Place the column into a 1.5 ml vial and avoid splashing any ethanol onto the spin filter.

15. Depending on downstream applications, add an appropriate amount of 1×TE. For maximum yield, add 50 ul 1×TE and do steps 16 and 17, then reload 50 ul eluted DNA on the column and redo steps 16 and 17, and then the final yield is 50 ul.

16. Incubate for 10 minutes at 65° C.

17. Spin at about 10,000 rpm for 30 seconds in a microcentrifuge and collect the eluted DNA in the 1.5 ml vial. Discard the column.

DNA in the tube is ready now, and I recommend storing DNA frozen (−20° C.).

DNA Extraction Protocol For Dechlorinating Cultures

(Qiagen DNeasy Blood & Tissue Kit, with Modifications)

1. Set the temperature on two incubators or water baths (one at 65° C., one at 37° C.).

2. Make pellets with 10 ml of culture, freeze overnight

3. Remove all of the supernatant from the solution

4. Add 180 μl Enzyme lysis buffer, mix by pipetting up and down (20 mM Tris.HCl, 2 mM EDTA, 250 ug/ml achromopeptidase, and 20 mg/ml of lysozyme. (Prepare fresh lysozyme, as it appears to be critical for efficient lysis and enzyme activity seems to decrease with storage)

5. Incubate at 37° C. for 60 minutes. Periodically check the incubations and flick tubes if necessary to keep cells in suspension.

6. Add SDS to 1.2% (w/v) and vortex briefly. Incubate at 56° C. for 10 minutes.

The suspension will clarify.
7. (Begin following step 4 of the Qiagen DNeasy Blood & Tissue kit pretreatment protocol for Gram positive bacteria) Add 25 μl proteinase K and 200 μl buffer AL (without ethanol) and vortex briefly. The suspension will clarify further.

8. Incubate at 56° C. for 30 minutes.

9. Spin the lysate at 10,000×g for 1 minute.

10. Check for any intact cell material or debris and remove the supernatant to a separate tube.

11. Add 200 μl ethanol (96-100%) and mix thoroughly by vortexing. Spin down briefly to remove lysate from the lid of the microcentrifuge tube.

For the following steps, be careful not to invert the spin column or otherwise spill ethanol-containing solutions around the walls of the spin column. Any ethanol layers embedded between the spin column and the collection tube are difficult to remove and may appear in the final eluate. Keep the collection tubes upright.

12. Carefully pipet (avoiding bubbling) the entire lysate onto the DNeasy spin column placed in a 2 ml collection tube (provided). Centrifuge at 8,000×g for 1 minute. Discard the flow-through and the centrifuge tube.

13. Place the spin column into a clean collection tube (provided), add 500 μl buffer AW1, and centrifuge at 8,000×g for 1 minute. Discard the flow-through and the centrifuge tube.

14. Place the spin column into a clean collection tube (provided), add 500 μl buffer AW2, and centrifuge at 17,000×g for 3 minutes to dry the DNeasy membrane. Discard the flow-through and the centrifuge tube.

15. Place the spin column into a clean microcentrifuge tube. Add 100 μl buffer AE to the membrane, let stand for 1 minute, and centrifuge at 10,000×g for 1 minute.

16. Apply the eluate back onto the same spin column and centrifuge again at 10,000×g for 1 minute.

Store DNA at −20° C.

### Experiments for Determining Kinetic Parameters

We carried out the experiments in triplicate batch reactors consisting of 160-mL glass serum bottles (100 mL liquid, 60 mL gas). Initially, 10% DehaloR̂2 inoculum from a well-grown culture was transferred to each bottle, along with 5 mM lactate and 11.1 mM methanol. We added 10 to 15 μL of neat TCE (˜1000 μmoles). The initial pH was between 7.2 and 7.5 and the initial bicarbonate concentration was 30 mM. After complete dechlorination, we removed 1.5 mL of culture for DNA extraction and 0.5 mL for protein assay (Bicinchoninic Acid Kit, Sigma-Aldrich). Before the next addition of lactate, methanol, and TCE, the bottles were flushed with N₂ to remove headspace gases and were amended with 10 mM bicarbonate. TCE and electron donors were added five consecutive times, until the rates of reductive dechlorination, the concentration of biomass, and the cell copies measured with qPCR stabilized and then started to decrease, indicating the onset of biomass decay. Table 12 shows a comparison of key kinetic parameters between DehaloR̂2 and select TCE to ethene mixed microbial communities. The shaded values are calculated and the non-shaded were determined experimentally. For DehaloR̂2 the second number in each box is the standard deviation.

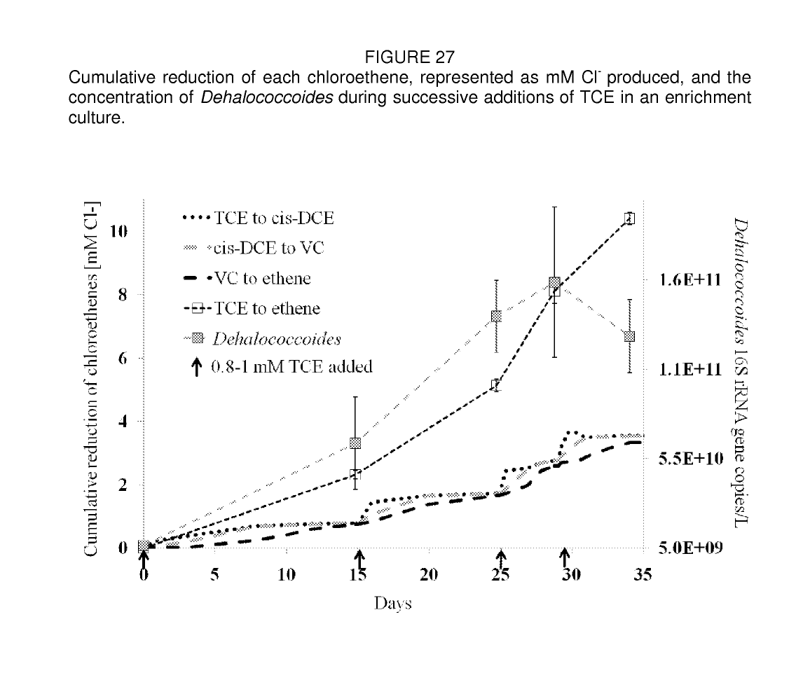

TABLE 12
The figure used to determine these rates is figure 27.

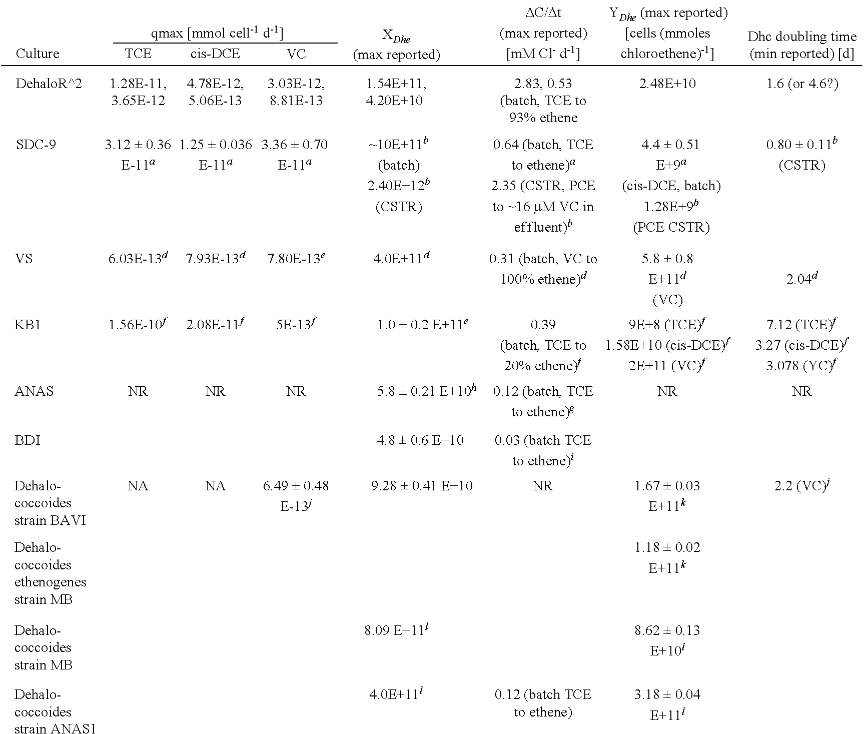

^{a}Shaefer et al. (2009a); calculated from mathematical model
^{b}Shaefer et al. (2009b); 4,000 L PCE CSTR with 2,500 L medium
^{c}Cupples et al. (2003); calculated from mathematical model
^{d}Cupples et al. (2004); qₘₐₓ values from mathematical model and doubling time from 1/μ
^{e}Duhamel et al. (2007)
^{f}Haest et al. (2010); qₘₐₓ values from mathematical model and doubling time from 1/μ where μ = qₘₐₓ x Y
^{g}Richardson et al. (2002), 25° C.
^{h}Richarson et al. (2005)
ⁱAmos et al. (2008)
^{j}He et al. (2003)
^{k}He et al. (2007)
^{l}Cheng and He (2009)

Clone Library and qPCR Analysis of DehaloR̂2 Community Structure

In order to identify the bacterial species in DehaloR̂2, we constructed a clone library of the highly enriched culture, resulting in the phylogenetic information shown in Table 13. Of the 73 clones, 53 (72.6%) were fermenters with homoacetogens constituting the largest fraction (35 clones, 47.9%) of which 31 were Acetobacterium and 4 were Spirochaetes (see Table S2 for additional information). Clostridium accounted for 14 clones (19.1%), of which 9 (12.3%) showed closest phylogenetic identity to Clostridium ganghwense, a lactate to propionate fermenter (Zhao et al. 2008) and 5 clones (6.8%) had a maximum match of 91% to the genebank, suggesting the presence of a new strain. Nineteen clones (26.0%) were Dehalococcoides sp., which appears to represented by several strains, some of which are novel, as suggested by the sequencing data. While the relative abundance of fermenters, including Acetobacterium, agreed well with the pyrosequencing analysis, we used qPCR to verify the abundance of Dehalococcoides compared to general bacteria and found 1.54E+11 (±4.20E+10 standard deviation) gene copies/L: 1.23E+12 gene copies/L (±7.15E+11). We also quantified Geobacteraceae, the family containing bacteria of the genus Geobacter; the concentration was 2.67E+10 genes/L (±5.10E+9). Desulforomonas and Dehalobacter, two other common TCE to cis-DCE dechlorinators, were not detected, even when using specific primers and nested PCR (data not shown), indicating absence of these bacteria. Given our current understanding of reductively dechlorinating anaerobes, the reductive dechlorination activity displayed by DehaloR̂2 appears to be linked to Dehalococcoides and Geobacteracea. Results from qPCR targeting vcrA, tceA and bvcA showed an abundance of these reductive dehalogenase genes, and predominance of tceA over the other dehalogenases: 1.56E+11 (±5.01E+10), 9.96E+10 (±5.08E+10), and 5.09E+9 (±9.74E+8) gene copies/L, respectively. Table 13 lists a Clone library of DehaloR̂2 from an enriched culture transferred for about one year.

TABLE 13

| Sequence | Closest Genbank Match | % | Number |
|---|---|---|---|
| Name | (as of Jun. 17, 2010) | Identity | of Clones |
| C07 | Dehalococcoides sp. MB | 99% | 13 |
| A10 | Dehalococcoides sp. MB | 99% | 1 |
| H06 | Dehalococcoides sp. MB | 95% | 1 |
| A02 | Dehalococcoides sp. MB | 97% | 1 |
| D10 | Dehalococcoides sp. MB | 99% | 1 |
| E08 | Dehalococcoides sp. MB | 99% | 1 |
| H04 | Dehalococcoides ethenogenes 195 | 98% | 1 |
| A07 | Acetobacterium wieringae strain | 98% | 31 |
| | DSM 1911 | | |
| B02 | Spirochaetes bacterium SA-8 | 99% | 4 |
| A04 | Uncultured Firmicutes bacterium clone | 97% | 2 |
| | CS-42-4 | | |
| F01 | Uncultured bacterium clone:TSBX14 | 98% | 2 |
| | Uncultured bacterium | 96% | |
| G01 | Bacterium enrichment culture clone LA60 | 99% | 9 |
| | Clostridium ganghwense strain HY-42-06 | 97% | |
| D07 | Bacterium enrichment culture clone LA60 | 91% | 5 |
| | Clostridium thermopalmarium strain BVP | 89% | |
| G05 | Sedimentibacter sp. C7 | 99% | 1 |
| TOTAL | | | 73 |

Kinetic Parameters of DenaloR̂2

Comparing kinetic parameters for reductive dechlorination is of practical value when selecting potential cultures for bioaugmentation. The results of an enrichment culture time-course experiment are presented in FIG. 27. We used this experiment to directly measure various chloroethene kinetic parameters, listed in Table 12.

The rate of TCE reductive dechlorination to ethene (ΔC/Δt) was calculated for each addition of TCE and the maximum rate, 2.83 mM Cl⁻ d⁻¹ was reached in the third addition. While this parameter provides an indication of the maximum degradation potential of the microbial community, it is not usually reported.

A more commonly reported estimate of degradation rates is the related kinetic parameter qₘₐₓ [mmoles cell⁻¹ d⁻¹], the maximum rate of substrate utilization. It is generally estimated by fitting data from batch reactor experiments to a Monod-based model (citations). Using data from the same time-course experiment as above, we sought to experimentally determined the actual achievable qₘₐₓ of Dehalococcoides for TCE, cis-DCE, and VC using the relationship:

q_{max,i}=(ΔCᵢ/Δt)ₘₐₓ/Xₐ₀, (1)

where (ΔCᵢ/Δt)ₘₐₓ is the maximum measured change in concentration between two time points for each chloroethene i, and Xₐ₀ is the Dehalococcoides concentration for the corresponding addition of TCE. Since the concentration of Dehalococcoides was greater than Geobacteracae, the measured values of q_{max,i} re representative of those of Dehalococcoides. The maximum values were all achieved between the first and second time points of the fourth addition of TCE.

Also of potential practical value are the doubling time, yield (Y_{Dhc}), and maximum achievable concentration of Dehalococcoides (X_{a,Dhc}). The doubling time is useful for assessing the length of time to reach the maximum rates of degradation, Y_{Dhc} is an indication of the resources required to reach those maximum rates, and X_{a,Dhc} is significant since up to a point there is a correlation between the concentration of Dehalococcoides and (ΔC/Δt)_{max,TCE to ethene}, and q_{max,i}.

### Fermentable Electron Donor/s Effect on Dehalogenating Culture Rates Over a Time-Course Experiment

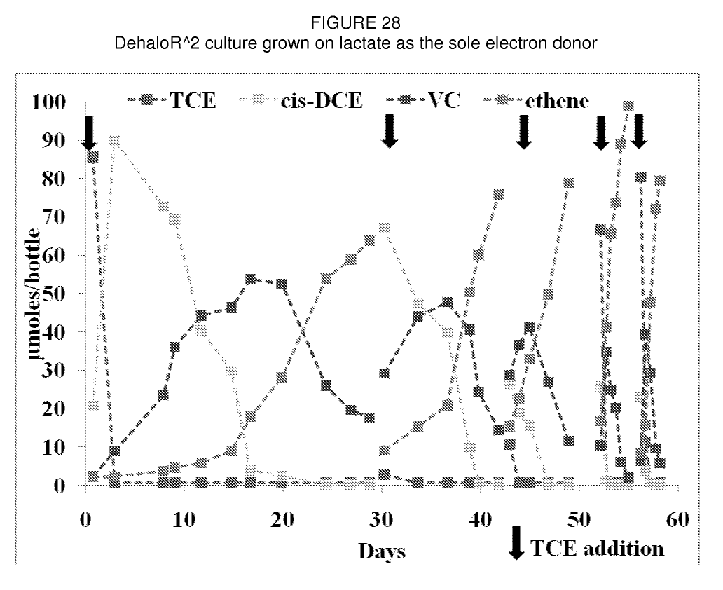

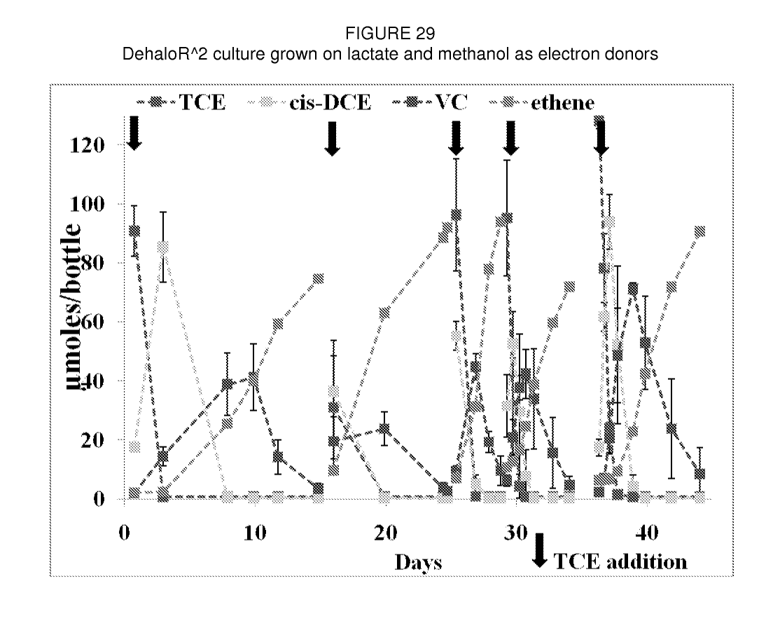

We previously showed that the combination of (lactate and methanol) fermentable substrates, enriched for the robust, fast dechlorinating culture, DehaloR̂2, and resulted in enhanced, efficient TCE to ethene dechlorination rates. Figure A and B represent a time-course experiment in triplicate batch serum bottles with TCE as electron acceptor and the same amount of electron equivalents provided as lactate, and lactate and methanol. The cultures were amended with a combination of 10 mM lactate (FIG. 28), and 5 mM lactate in combination with 11.1 mM methanol (FIG. 29). TCE was added to both sets of reactors five consecutive times. The batch cultures were reamended with electron donors whenever TCE was added and whenever dechlorination rates slowed down, a sign that there was a hydrogen limitation for the dechlorinating populations. We determined that the combination of lactate and methanol yielded faster dechlorination rates over a shorter period of time and required less electron equivalents to carry out complete dechlorination.

### Removal of Headspace Gases to Adjust pH for Enhanced Dechlorination Rates

Fermentation reactions provide a source of H₂ to the culture, H₂ is the sole electron donor for Dehalococcoides populations in DehaloR̂2. Proton and CO₂-producing fermentation, and dechlorination reactions decrease the pH of the medium while methanogenesis and acetogenesis increase the pH by consuming protons and CO₂— Improper balance of these reactions negatively impact a culture's performance in terms of complete and fast conversion of TCE to ethene.

The following equations show the effect of pH

Fermentable substrates→XHCO₃⁻+YCO₂+ZH+ (1)

HCO₃⁻+4H₂+H⁺→CH₄+3H₂O (2)

2HCO₃⁻+4H₂+H⁺→CH₃COOH+4H₂O (3)

C₂HCl₃+3H₂→C₂H₄+3Cl⁻+3H⁺ (4)

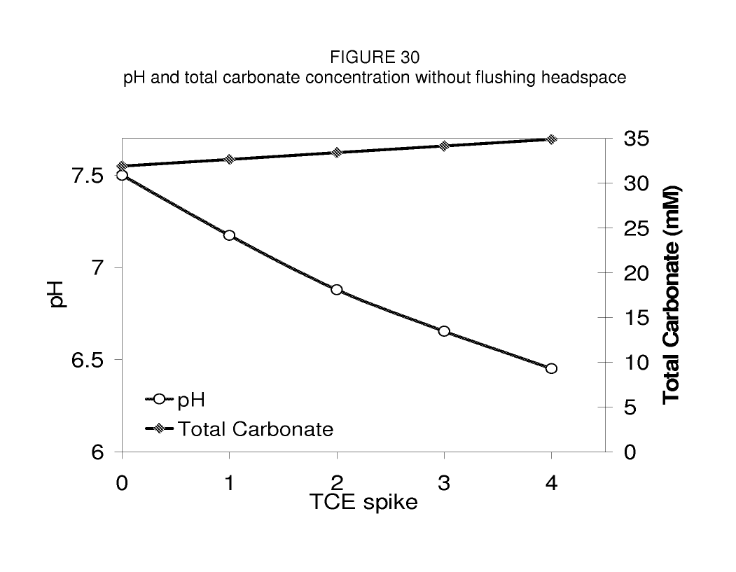

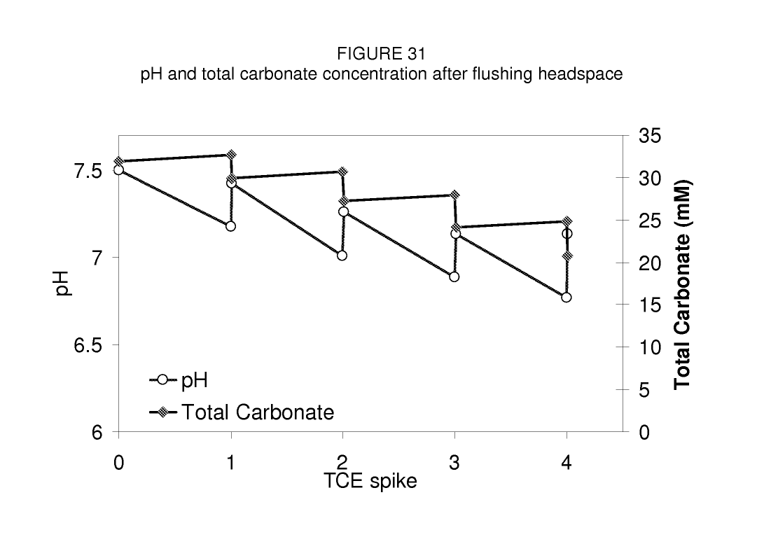

In FIGS. 30 and 31, we model the pH trends and the fate of total carbonate in our batch reactors containing 30 mM sodium bicarbonate as buffer plus lactate and methanol (fermentable substrates), as electron donors. When fermentables and TCE are added to the cultures, the pH of the medium decreases to 6.4 over 4 TCE spikes (additions), a pH no longer within optimum values for dechlorination. In addition, the total carbonate increases slightly in the system. If some of the CO₂ and as an effect total alkalinity is removed after each TCE spike, the pH of the medium increases (as an effect of reactions 2 and 3) and is maintained within optimum values after each TCE spike.

To remove some of the CO₂ and adjust the pH in our reactors, we developed a method in which we flush the headspace of the serum bottles with ultra high purity nitrogen. In order to do so, we insert a nitrogen line with a 21-gauge needle into the rubber stopper of the serum bottle and an addition 21-gauge needle for venting. The bottles are flushed for 10-15 minutes, a time sufficient to replace headspace gases with nitrogen. The method has proven crucial in achieving fast dechlorination rates of TCE reduction for DehaloR̂2.

### REFERENCES

1. Abelson P H. 1990. Science 250:733.
2. Abrahamsson K, et al. 1995. Limnology and Oceanography 40(7):1321-1326.
3. Adolfsson-Erici M, et al. 2002. Chemosphere 46:1485-1489.
4. Amos B K, et al. 2007. Environmental Science & Technology 41(5):1710-1716.
5. Amos B K, et al. 2008. Environmental Science & Technology 42(15):5718-5726.
6. Agency for Toxic Substances and Disease Registry 2007. ToxFAQs; http://www.atsdr.cdc.gov/toxfaq.html. Atlanta, Ga.
7. Audu A A, Heyn A H A. 1988. Water Research 22(9):1155-1162.
8. Aulenta F, et al. 2006. Journal of Chemical Technology and Biotechnology 81(9):1463-1474.
9. Ballapragada B S, et al. 1997. Environ. Sci. Technol. 31:1728-1734.
10. Bester K. 2003. Water Research 37:3891-3896.
11. Boehncke A, et al. 2003. International Programme on Chemical Safety (IPCS), Geneva:1-68.
12. Bouchard B, et al. 1996. International Journal of Systematic Bacteriology 46(4):1010-1015.
13. Bradley P M. 2000. Hydrogeol. Journal 8:104-111.
14. Breneman D L, et al. 2000. Cutis; Cutaneous Medicine for the Practitioner 66:296-300.
15. Chalew T E A, Halden R U. 2009. Journal of the American Water Resources Association 45(1):4-13.
16. Chang Y C, et al. 2000. Journal of Bioscience and Bioengineering 89(5):489-491.
17. Chen J G, et al. 2008. Endocrinology 149(3):1173-1179.
18. Christof O, et al. 2002. Biogeochem 59:143-160.
19. Chung J, et al. 2008. Environ. Sci. Technol. 42(2):477-483.
20. TCC Consortium. 2002. High Production Volume (HPV) Chemical Challenge Program Data Availability and Screening Level Assessment for Triclocarban: http://www.epa.gov/chemrtk/pubs/summaries/tricloca/c14186tp.pdf.
21. Coogan M A, et al. 2007. Chemosphere 67(10):1911-1918.
22. Costanza J, Pennell K D. 2007. Environmental Science & Technology 41(5):1729-1734.
23. Cupples A M, et al. 2003. Applied and Environmental Microbiology 69(2):953-959.
24. Cupples A M, et al. 2004. Environ. Sci. Technol. 38:1102-1107.
25. Darlington R, et al. 2008. Environmental Science & Technology 42(12):4323-4330.
26. De Rooij C, et al. 1998. Environ Monit Assess 52:489-508.
27. De Wildeman S, et al. 2003. Biodegradation 14(4):241-247.
28. Dennis P C, et al. 2003. Canadian Journal of Microbiology 49(1):15-27.
29. Deweerd K A, et al. 1990. Archives of Microbiology 154(1):23-30.
30. Diekert G. CO2 reduction to acetate in anaerobic-bacteria; 1990. p 391-395.
31. Dimarco A A, et al. 1990. Annual Review of Biochemistry 59:355-394.
32. Dokianakis S N, et al. 2004. Water Sci. Technol. 50(5):341-346.
33. Doong R A, Wu Y W. 1997. Chemosphere 34(8):1653-1662.
34. Duhamel M, Edwards E A. 2006. Fems Microbiology Ecology 58(3):538-549.
35. Duhamel M, et al. 2004. Applied and Environmental Microbiology 70(9):5538-5545.
36. Ellis D E, et al. 2000. Environmental Science & Technology 34(11):2254-2260.
37. EPA US. 2003. TSCA Chemical Substances Inventory. Washington D.C.
38. U.S. Environmental Protection Agency. 2007. Chemical Properties for SSL Development: www.epa.gov/superfund/health/conmedia/soil/pdfs/attachc.pdf. Washington, D.C.
39. U.S. Environmental Protection Agency. 2008a. National Priorities List (NPL): http://www.epa.gov/superfund/sites/npl/. Washington, D.C.
40. U.S. Environmental Protection Agency. 2008b. Reregistration Eligibility Decision and Risk Assessment for the Pesticidal Uses of Triclosan: http://www.epa.gov/oppsrrd1/REDs/factsheets/triclosan_fs.htm. Washington, D.C.
41. U.S. Environmental Protection Agency. 2009. Integrated Risk Information System: http://cfpub.epa.gov/ncea/iris/index.cfm. Washington, D.C.
42. Fathepure B Z, Boyd S A. 1988. Fems Microbiology Letters 49(2):149-156.
43. Federle T W, et al. 2002. Environmental Toxicology and Chemistry 21(7):1330-1337.
44. Field J A, Sierra-Alvarez R. 2004. Rev Environ Sci Biotechnol 3:184-254.
45. Freedman D L, Gossett J M. 1989. Applied and Environmental Microbiology 55(9):2144-2151.
46. Friis A K, et al. 2007a. Water Research 41(2):355-364.
47. Friis A K, et al. 2007b. Biodegradation 18(6):661-674.
48. Gledhill W E. 1975. Water Research 9(7):649-654.
49. Glod G, et al. 1997. Environmental Science & Technology 31(1):253-260.
50. Gribble G W. 1994. Am J Public Health 84:1183.
51. Gribble G W. 2003. Chemosphere 52:289-297.
52. Griffin B M, et al. 2004. Environmental Science & Technology 38(16):4300-4303.
53. Halden R U, Paull D H. 2004. Environmental Science & Technology 38(18):4849-4855.
54. Halden R U, Paull D H. 2005. Environmental Science & Technology 39(6):1420-1426.
55. Hartmans S. 1995. Microbial degradation of vinyl chloride. Singh V P, (ed.), editors. Amsterdam, Netherlands: Elsevier Science.
56. He J, Löffler FE. 2003. Isolation of a vinyl chloride-respiring population in pure culture. Abstracts of the General Meeting of the American Society for Microbiology 103:Q-016.
57. He J, Sung et al. 2005a. Environmental Microbiology 7(9):1442-1450.
58. He J, et al. 2005b. Environmental Microbiology 7(9):1442-1450.
59. He J Z, et al. 2003a. Applied and Environmental Microbiology 69(2):996-1003.
60. He J Z, et al. 2003b. Applied and Environmental Microbiology 69(2):996-1003.
61. He J Z, et al. 2003c. Nature 424(6944):62-65.
62. He J Z, et al. 2003d. Nature 424(6944):62-65.
63. He J Z, et al. 2006. Environmental Science & Technology 40(14):4429-4434.
64. Heidler J, Halden R U. 2007. Chemosphere 66(2):362-369.
65. Heidler J, et al. 2006. Environmental Science & Technology 40(11):3634-3639.
66. Hendrickson E R, et al. 2002. Applied and Environmental Microbiology 68(2):485-495.
67. Heron G, et al. 2005. Ground Water Monit R 25(4):92-107.
68. Holliger C, et al. 1998a. Archives of Microbiology 169(4):313-321.
69. Holliger C, et al. 1992. Journal of Bacteriology 174(13):4427-4434.
70. Holliger C, et al. 1998b. Fems Microbiology Reviews 22(5):383-398.
71. Holmes V F, et al. 2006. Applied and Environmental Microbiology 72(9):5877-5883.
72. Holscher T, et al. 2004. Applied and Environmental Microbiology 70(9):5290-5297.
73. Hood E D, et al. 2008. Ground Water Monitoring and Remediation 28(2):98-107.
74. Johnson R, et al. 1963. Pediatrics 31:222-225.
75. Keppler F, et al. 2002. Environmental Science & Technology 36(11):2479-2483.
76. Kittelmann S, Friedrich M W. 2008a. Environmental Microbiology 10:31-46.
77. Kittelmann S, Friedrich M W. 2008b. Environmental Microbiology 10(6):1557-1570.
78. Kittelmann S, Friedrich M W. 2008c. Environmental Microbiology 10(6):1557-1570.
79. Kolpin D W, et al. 2002. Environmental Science & Technology 36(6):1202-1211.
80. Krajmalnik-Brown R, et al. 2004. Applied and Environmental Microbiology 70(10):6347-6351.
81. Kube M, et al. 2005. Nature Biotechnology 23(10):1269-1273.
82. Löffler F E, et al. 1997. Applied and Environmental Microbiology 63(12):4982-4985.
83. Löffler F E, et al. 2005. Environmental Microbiology. p 77-111.
84. Levy C W, et al. 1999. Nature 398:383-384.
85. Loffler F E, Edwards E A. 2006. Current Opinion in Biotechnology 17(3):274-284.
86. Lomans B P, et al. 1997. Applied and Environmental Microbiology 63(12):4741-4747.
87. Luijten M, et al. 2003. International Journal of Systematic and Evolutionary
Microbiology 53:787-793.
88. Lyimo T J, et al. 2002. Ambio 31(7-8):614-616.
89. Müller J A et al. 2004. Applied and Environmental Microbiology 70(8):4880-4888.
90. Magnuson J K, et al. 2000. Appl. Environ. Microbiol. 66:5141-5147.
91. Major D W, et al. 2002. Environmental Science & Technology 36(23):5106-5116.
92. Maymo-Gatell X, et al. 1997. Science 276(1568-1571).
93. Mazur C S, Jones W J. 2001. Environ Sci Technol 35:4783-4788.
94. McAvoy D C, et al. 2002. Environmental Toxicology and Chemistry 21:1323-1329.
95. McCarty P L. 1997. Science 276:1521-1522.
96. McCarty P L, et al. European Journal of Soil Biology 43(5-6):276-282.
97. McMurray L M et al. 1998. Nature 394:531-532.
98. Metje M, Frenzel P. 2005. Applied and Environmental Microbiology 71(12):8191-8200.
99. Miller G S et al. 2005. Environmental Science & Technology 39(8):2631-2635.
100. Miller T R, et al. 2008. Environmental Science & Technology 42(12):4570-4576.
101. Nolen G, Dierckman T. 1979. Toxicol. Appl. Pharmacol. 51:417-425.
102. Oremland R S, Capone D G. 1988. Advances in Microbial Ecology 10:285-383.
103. Orvos D R, et al. 2002. Environ. Toxicol. Chem. 21(1338-1349).
104. Ponte C, et al. 1974. Sem. Hop. Paris 50:359-365.
105. Ritalahti K M, et al. 2006. Applied and Environmental Microbiology 72(4):2765-2774.
106. Ritalahti K M, et al. 2005. Ind. Biotechnol. 1:114-118.
107. Rosalind A. et al. 2005. Environmental Toxicology 20(2):160-164.
108. Roy R, et al. 1997. Fems Microbiology Ecology 24(4):311-320.
109. Sanford R A, et al. 1996. Applied and Environmental Microbiology 62(10):3800-3808.
110. Schaefer C E, et al. 2009. Chemosphere 75(2):141-148.
111. Scheutz C, et al. 2008. Environmental Science & Technology 42(24):9302-9309.
112. Scholzmuramatsu H, et al. 1995. Archives of Microbiology 163(1):48-56.
113. Sharma P K, McCarty P L. 1996. Applied and Environmental Microbiology 62(3):761-765.
114. Sipma J, et al. 2003. Fems Microbiology Ecology 44(2):271-277.
115. 2009. http://www.siremlab.com/casestudies.html.
116. Sparling R, Daniels L. 1987. Canadian Journal of Microbiology 33(12):1132-1136.
117. Stasinakis A S, et al. 2007. Chemosphere 68(2):375-381.
118. Stupperich E. 1993. Fems Microbiology Reviews 12(4):349-366.
119. Sutler M T E, Russell A D. 1999. Journal of Hospital Infection 43(4):281-291.
120. Sung Y. 2005a. Isolation and ecology of bacterial populations involved in reductive dechlorination of chlorinated solvents. Atlanta: Georgia Institute of Technology.
121. Sung Y. 2005b. Isolation and ecology of bacterial populations involved in reductive dechlorination of chlorinated solvents. Atlanta: Georgia Institute of Technology.
122. Sung Y, et al. 2006a. Applied and Environmental Microbiology 72(4):2775-2782.
123. Sung Y, et al. 2006b. Applied and Environmental Microbiology 72(3):1980-1987.
124. Sung Y, et al. 2003. Applied and Environmental Microbiology 69(5):2964-2974.
125. Thompson A, et al. 2005. Water Environment Research 77(1):63-67.
126. Veldhoen N, et al. 2006. Aquatic Toxicology 80(3):217-227.
127. Walsh S E, et al. 2003. Journal of Applied Microbiology 94(2):240-247.
128. U.S. EPA, Office of Research and Development. 2006 http://www.epa.gov/ATHENS/learn2model/part-two/onsite/esthenry.htm.
129. Wild A P, et al. 1995. Biodegradation 6(4):309-318.
130. Wu Q Z, et al. 2000. Applied and Environmental Microbiology 66(1):49-53.
131. Yackovich F, et al. 1986. J. Soc. Cosmet. Chem. 37:99-104.
132. Ying G-G, et al. 2007. Environmental Pollution 150(3):300-305.
133. Yu J T, et al. 2006. Agricultural Water Management 86(1-2):72-80.

1. An composition for dehalogenation of a sample that is contaminated with at least one halogenated chemical comprising a microbial consortium of a mixture of microbial strains of Chloroflexy, Firmicutes and Proteobacteria.

2. A composition for dehalogenation of a sample that is contaminated with at least one halogenated chemical comprising an anthropogenically produced or harvested microbial consortium of a mixture of microbial strains of Chloroflexy, Firmicutes and Proteobacteria.

3. The composition of claim 1 or claim 2, wherein the consortium further comprises one or more microorganisms selected from the group consisting of Spirochaetes, Delta proteobacteria, Beta proteobacteria, Gamaproteobacteria, Acetobacterium, Acidaminobacter, Sedimentibacter, Gracilibacter, and Clostridium.

4. The composition of claim 1 or claim 2, wherein the consortium comprises at least two strains from the group of microorganisms comprising Trichlorobacter, Geobacter, Clostridium, Acetobacterium, Spirochaetes and Dehalococcoides.

5. A microbial composition according to claim 1 or claim 2, wherein the consortium comprises Dehalococcoides.

6. A microbial composition for concurrent dehalogenation of a mixture of halogenated ethenes comprising a naturally occurring dehalogenating microbial species, wherein said microbial species comprises at least one 16S rDNA nucleic acid sequence that has more than 95% identity to a nucleic acid sequence consisting of SEQ ID NO: 4, a nucleic acid sequence that when translated into protein has more than 80% identity to a nucleic acid sequence consisting of SEQ ID NO: 1, a nucleic acid sequence that when translated into protein has more than 80% identity to a nucleic acid sequence consisting of SEQ ID NO:2, a nucleic acid sequence that when translated into protein has more than 80% identity to a nucleic acid sequence consisting of SEQ ID NO:3, or a nucleic acid sequence consisting of SEQ ID NO:4.

7. A microbial composition according to claim 6, wherein the consortium further comprises at least one chloroethene reductase nucleic acid sequence that has more than 80% identity to the group of chloroethene reductases comprised of TceA, BvcA and VcrA.

8. A method for dehalogenating a chemical composition comprising organohalides comprising contacting said chemical composition with the microbial composition of any of claim 1, 2, 3, 4, or 6; and concurrently anaerobically dehalogenating said organohalides in said composition.

9. A method according to claim 9, wherein the organohalide comprises at least one of trichloroethene, cis-1,2-dichloroethene; trans-1,2-dichloroethene, vinyl chloride, 1,1-dichloroethene, or tetrachloroethene.

10. A method for dehalogenating halogenated waste, comprising: contacting at least one organohalogen with a laboratory cultured/enriched bioremediative consortium comprising strains of microorganism comprising Chloroflexy, Spirochaetes, Firmicutes, Proteobacteria.

11. A method according to claim 10, wherein the halogenated waste is taken from the group comprising contaminated soil, contaminated sediment, contaminated water, contaminated industrial wastewater, contaminated domestic wastewater, contaminated sewage sludge, contaminated biosolids.

12. A method of producing a microbial dehalogenating consortium comprising culturing microbes in an anaerobic medium with at least one chlorinated ethene and an electron donor, a sediment sample obtained from a site contaminated with a mixture of chlorinated antimicrobials.

13. The method of any of claims 10-12, wherein said dehalogenating comprises debromination, deiodination, defluorination or dechlorination of organohalogens.

14. The method of any of claims 10-12, wherein said method produces the dechlorination of mono-, di-, tri-, and polychlorinated aliphatics.

15. The method of any of claims 10-12, wherein said method produces the dechlorination of mono-, di-, tri-, and polychlorinated aromatics.

16. The method of any of claims 10-12, wherein said method produces the dehalogenation of mixtures of organohalogens comprising at least two organohalides, comprising fluorinated organics, chlorinated organics, brominated organics, and ionidated organics.

17. A method of performing dehalogenation of organohalogens other than antimicrobials in the presence of antimicrobial agents comprising contacting said organohalogens with a composition of any of claim 1, 2, 3 or 4.

18. A method of performing dehalogenation of antimicrobial agents comprising contacting said antimicrobial agent with a composition of any of claim 1, 2, 3 or 4.

19. The method of claim 18, wherein said antimicrobial agent is an aromatic antimicrobial agent.

20. The composition of claim 1 or claim 2 wherein the composition is formulated as a microbial conglomerate, floc, biofilm pellet or bead.

21. The composition of claim 1 or claim 2 that is resistant to the presence of antimicrobial compounds.

22. The composition of claim 1 or claim 2 that is resistant to elevated levels of chloroethenes.

23. A method for enriching in the laboratory for dehalogenating microbial mixed cultures able to dehalogenate a chemical composition comprising organohalides comprising contacting said chemical composition with the microbial composition of any of claim 1, 2, 3, 4 or 6; and concurrently anaerobically dehalogenating said organohalides in said composition.

24. A composition of fermentable substrates for fast dehalogenating cultures comprising fermentable substrates enriched for dechlorinating culture, DehaloR̂2, wherein TCE is introduced into the enriched fermentable substrates as an electron acceptor and wherein the cultures were amended with a combination of lactate and methanol.

25. A method to adjust pH and to remove headspace gases in culture vessels in order to optimize dehalogenation of a culture for faster degradation rates of a chemical compound comprising the steps of:
initiating a fermentation reaction in a reactor to generate a source of proton and CO₂-producing fermentation, and dechlorination reactions;
adding fermentables and TCE to the culture;
monitoring the pH of the culture to determine when the pH reaches a predeteremined value not optimal for dechlorination; and
removing CO₂ so as to adjust the pH in the reactor by flushing the headspace of the culture vessel with a gas taken from the group comprising nitrogen and noble gases until the pH changes to a value that promotes dechlorination.

26. The method of claim 25 wherein the step of removing CO₂ is implemented when the pH decreases to 6.4.

27. A large scale production method for the production of a composition of claim 1 or claim 2 comprising growing said microbial consortium according to a method of claim 25.
